(19) United States
(12) Patent Application Publication
(10) Pub. No.: US 2018/0292408 A1
(43) Pub. Date: Oct. 11, 2018

(54) CAMELID SINGLE-DOMAIN HCV ANTIBODIES AND METHODS OF USE

(71) Applicant: QOOLABS, INC., San Diego, CA (US)

(72) Inventor: Hong QI, San Diego, CA (US)

(73) Assignee: QOOLABS, INC., San Diego, CA (US)

(21) Appl. No.: 15/567,353

(22) PCT Filed: Apr. 19, 2016
(86) PCT No.: PCT/US16/28312

§ 371 Date: Oct. 17, 2017

Related U.S. Application Data
(60) Provisional application No. 62/150,212, filed on Apr. 20, 2015.

(51) Int. Cl. G01N 33/576; C07K 14/005; G01N 33/68; C07K 16/10

(52) U.S. Cl. CPC G01N 33/5767; C07K 14/005; G01N 33/6854; C07K 16/109; C12N 2770/24222; C07K 2317/94; C07K 2319/20; G01N 2333/186; C07K 2317/22; C07K 2317/569; C07K 2317/24; C12N 2770/24231

(57) ABSTRACT

The present disclosure in some aspects relates to HCV core antigen polypeptides. In some aspects, the present disclosure further relates to HCV antibodies, including camelid antibodies that specifically bind to HCV core antigen, and antibody fragments. The disclosure further relates to methods of detecting an analyte in a sample using a camelid antibody, such as a camelid VHH antibody or fragments thereof. In one aspect, provided herein is a technology platform for isolating highly specific antibodies and applying these antibodies in an immunoassay, such as a lateral flow immunoassay (LFIA). In some aspects, this technology is used to develop HCV core antigen specific antibodies and to produce LFIA devices for rapid and early diagnosis of HCV. In other aspects, a rapid test is provided for screening and detection of hepatitis C virus infection to improve the diagnosis rate and effectively prevent HCV infection transmission.

### CROSS-REFERENCE TO RELATED APPLICATIONS

This application claims priority to U.S. Provisional Patent Application Ser. No. 62/150,212, filed Apr. 20, 2015, entitled “Camelid Single-Domain HCV Antibodies and Methods of Use,” the content of which is incorporated herein by reference in its entirety for all purposes.

### STATEMENT OF RIGHTS TO INVENTIONS MADE UNDER FEDERALLY SPONSORED RESEARCH

This invention was made with the support by the U.S. Department of Health and Human Services, Centers for Disease Control and Prevention (CDC) Contract Number 200-2014-M-60477. The U.S. government may have certain rights in this invention.

### FIELD

The present disclosure in some aspects relates to HCV core antigen polypeptides. In some aspects, the present disclosure further relates to HCV antibodies, including camelid antibodies that specifically bind to HCV core antigen polypeptides, and antibody fragments. The disclosure further relates to methods of detecting an analyte in a sample using a camelid antibody, such as a camelid VHH antibody or fragments thereof.

### BACKGROUND

Hepatitis C virus (HCV) is an enveloped, single stranded RNA virus which infect human and causes hepatitis C. HCV infects about 3% of the world's population and most people are unaware of their infection status. About 80% of individuals with HCV infection develop chronic hepatitis and many will progress to have cirrhosis and hepatocellular carcinoma (HCC). See Shepard et al., Global epidemiology of hepatitis C virus infection, Lancet Infect Dis, 2005, 5(9):558-67; and WHO, Hepatitis C, Oct. 1, 2013, available from who.int/csr/disease/hepatitis/Hepc.pdf. HCV-related end stage liver disease is the leading reason for liver transplantation in the USA.

Hepatitis C infection is blood borne and is usually spread by sharing infected needles with a carrier, from receiving infected blood, or from accidental exposure to infected blood. All HCV positive persons are potentially infectious. CDC has issued recommendations for prevention and control of HCV infection and HCV related chronic disease. CDC, Hepatitis C, Oct. 1, 2013, available from cdc.gov/hepatitis/HCV/Management.htm. Because symptoms are generally absent in individuals with chronic HCV infection, recognition of infection requires risk factor screening to link with appropriate HCV testing. See Ghany et al., Diagnosis, management, and treatment of hepatitis C: an update, Hepatology, 2009, 49(4):1335-74. An early diagnosis in the course of the disease not only increases the chances of successful treatment, but also effectively limits further transmission of HCV.

There is a need for rapid tests that can be used for screening purposes, because such will further improve the diagnosis rate and effectively prevent HCV infection transmission. Unfortunately, there are no rapid tests currently available for speedy diagnosis of HCV infection.

### SUMMARY

In one aspect, disclosed herein is an isolated polypeptide comprising a hepatitis C virus (HCV) core antigen polypeptide comprising the amino acid sequence set forth in SEQ ID NO: 1, wherein the polypeptide does not comprise a full length natural HCV core antigen. In one aspect, disclosed herein is an isolated hepatitis C virus (HCV) core antigen polypeptide comprising the amino acid sequence set forth in SEQ ID NO: 1, wherein the polypeptide does not comprise a full length natural HCV core antigen. In one aspect, the polypeptide is not part of a hepatitis C virus. In another aspect, the polypeptide is not associated with a hepatitis C virus protein or polynucleotide. In one embodiment, the polypeptide is a part of a fusion polypeptide.

In one aspect, disclosed herein is an isolated polypeptide comprising an HCV core antigen fragment comprising any one of the amino acid sequences set forth in SEQ ID NOs: 2-11, or any combination thereof, wherein the polypeptide does not comprise a full length natural HCV core antigen. In another aspect, disclosed herein is an isolated HCV core antigen fragment comprising any one of the amino acid sequences set forth in SEQ ID NOs: 2-11, or any combination thereof, wherein the polypeptide does not comprise a full length natural HCV core antigen. In one embodiment, the polypeptide further comprises a tag sequence. In one aspect, the polypeptide is not part of a hepatitis C virus. In another aspect, the polypeptide is not associated with a hepatitis C virus protein or polynucleotide.

In any of the preceding embodiments, the polypeptide can comprise or can be conjugated to a detectable label. In one aspect, the detectable label is a colorimetric, a radioactive, an enzymatic, a luminescent and/or a fluorescent label. In any of the preceding embodiments, the detectable label can be a soluble label or a particle (such as a nanoparticle or a microparticle) or particulate label.

In any of the preceding embodiments, the polypeptide can be attached to a solid surface, such as a blot, a membrane, a sheet, a paper, a bead, a particle (such as a nanoparticle or a microparticle) (such as a nanoparticle or a microparticle), an assay plate, an array, a glass slide, a microtiter, or an ELISA plate.

In certain aspects, disclosed herein is a polynucleotide which encodes the polypeptide of any of the preceding embodiments, or a complimentary strand of the polynucleotide. In one aspect, the polynucleotide is codon-optimized for expression in a non-human organism or a cell. In one aspect, the organism or cell is a virus, a bacterium, a yeast cell, a plant cell, an insect cell or a mammalian cell.

In any of the preceding embodiments, the polynucleotide and/or the complimentary strand thereof can be DNA or RNA. In one aspect, the polynucleotide comprises the nucleotide sequence of SEQ ID NO: 12.

In certain aspects, disclosed herein is a vector comprising the polynucleotide of any of the preceding embodiments, or a complimentary strand thereof. In one aspect, the polynucleotide comprised in the vector further comprises a promoter sequence. In any of the preceding embodiments, the polynucleotide comprised in the vector can further encode a tag sequence. In any of the preceding embodiments, the polynucleotide comprised in the vector can comprise a poly-A sequence. In any of the preceding embodiments, the polynucleotide comprised in the vector can comprise a translation termination sequence.

In certain aspects, disclosed herein is a non-human organism or a cell transformed with the vector of any of the preceding embodiments. In one aspect, the non-human organism or cell is a virus, a bacterium, a yeast cell, a plant cell, an insect cell or a mammalian cell.

In certain aspects, disclosed herein is a method of recombinantly making a polypeptide, which method comprises culturing the organism or cell of any of the preceding embodiments, and recovering the polypeptide from the organism or cell. In one aspect, the method further comprises isolating the polypeptide, optionally by chromatography.

In certain aspects, disclosed herein is a polypeptide produced by the method of any of the preceding embodiments. In one aspect, the polypeptide comprises a native glycosylation pattern. In any of the preceding embodiments, the polypeptide can comprise a native phosphorylation pattern. In any of the preceding embodiments, the polypeptide can comprise one or more other post translational modifications, such as dephosphorylation, proteolysis, glycosylation, methylation, acetylation, citrullination, butyrylation, crotonylation, ubiquitination, and proline cis-trans isomerization. In any of the preceding embodiments, the polypeptide can comprise one or more disulfide bonds.

In some aspects, disclosed herein is a kit for detecting an antibody that specifically binds to an HCV core antigen polypeptide, which kit comprises, in a container, the polypeptide of any of the preceding embodiments.

In some aspects, disclosed herein is a method for detecting an antibody that specifically binds to an HCV core antigen polypeptide in a sample, which method comprises contacting the polypeptide of any of the preceding embodiments with the sample and detecting a polypeptide-antibody complex formed between the polypeptide and the HCV core antigen polypeptide in the sample to assess the presence, absence and/or amount of the antibody that specifically binds to an HCV core antigen polypeptide in the sample. In one aspect, the sample is from a mammal. In one embodiment, the mammal is a human. In any of the preceding embodiments, the sample can be a biological sample, such as a plasma sample, serum sample, dried blood spot, urine sample, tissue sample, and buccal swab.

In any of the preceding embodiments, the method can be used for diagnosis, prognosis, stratification, risk assessment, and/or treatment monitoring of an HCV infection. In any of the preceding embodiments, the sample can be selected from the group consisting of a whole blood sample, a serum, a plasma, a urine and a saliva sample. In any of the preceding embodiments, the sample can be a clinical sample. In any of the preceding embodiments, the polypeptide-antibody complex is assessed by a sandwich or competitive assay format, optionally with a binder or antibody. In any of the preceding embodiments, the binder or antibody can be attached to a surface and functions as a capture binder or antibody. In any of the preceding embodiments, at least one of the binders or antibodies can be labeled. In any of the preceding embodiments, the polypeptide-antibody complex can be assessed by a format selected from the group consisting of an enzyme-linked immunosorbent assay (ELISA), immunoblotting, immunoprecipitation, radioimmunoassay (RIA), immunostaining, latex agglutination, indirect hemagglutination assay (IHA), complement fixation, indirect immunofluorescent assay (IFA), nephelometry, flow cytometry assay, plasmon resonance assay, chemiluminescence assay, lateral flow immunoassay, g-capture assay, inhibition assay and avidity assay.

In any of the preceding embodiments, the polypeptide-antibody complex can be assessed in a homogeneous or a heterogeneous assay format.

In one aspect, disclosed herein is an isolated camelid antibody that specifically binds to an epitope within an HCV core antigen polypeptide. In one aspect, the isolated camelid antibody is derived from a camel, a llama, an alpaca (Vicugna pacos), a vicuña (Vicugna vicugna), or a guanaco (Lama guanicoe). In one embodiment, the camel is a dromedary camel (Camelus dromedarius), a Bactrian camel (Camelus bactrianus), or a wild Bactrian camel (Camelus ferus). In any of the embodiments disclosed herein, multiple antibodies to multiple epitopes can be used. For example, multiple antibodies from the same camelid species to multiple different epitopes on the same or different antigens can be used. In other embodiments, multiple antibodies from different species (such as from different camelid species) can be used, and the antibodies can be specific to the same epitope, or specific to different epitopes on the same or different antigens.

In any of the preceding embodiments, the isolated camelid antibody can be a polyclonal antibody, a monoclonal antibody, an antibody fragment or a single-domain heavy-chain (VHH) antibody. In one embodiment, the VHH antibody is a llama VHH antibody.

In any of the preceding embodiments, the isolated camelid antibody can specifically bind to an epitope within an HCV core antigen polypeptide from a genotype selected from the group consisting of 1, 1a, 1a/1b, 1b, 2, 2a, 2a/2c, 2b, 3a, 3k, 4, 4a, 4a/4c, 4c/4d, 4c/4d/4e, 5/5a, 6a, and 6i.

In any of the preceding embodiments, the isolated camelid antibody can specifically bind to an epitope within the polypeptide comprising the amino acid sequence set forth in SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, or SEQ ID NO: 11, or any combination thereof. In one aspect, the isolated camelid antibody is produced by a process that comprises the steps of: a) immunizing a camelid with a polypeptide comprising the amino acid sequence set forth in SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, or SEQ ID NO:11, or any combination thereof; and b) recovering the antibody from the camelid. In one embodiment, the camelid is a llama.

In any of the preceding embodiments, the isolated camelid antibody can specifically bind to the HCV core antigen polypeptide. In any of the preceding embodiments, the isolated camelid antibody can be a part of a fusion polypeptide. In one aspect, the fusion polypeptide comprises a variable region of a camelid antibody and a constant region of a non-camelid antibody. In one embodiment, the fusion polypeptide comprises a variable region of a llama antibody and a constant region of a non-camelid antibody. In one embodiment, the fusion polypeptide comprises a variable region of a llama antibody and a constant region of a rabbit antibody. In one embodiment, the fusion polypeptide is a fusion llama VHH antibody that comprises a variable region of the llama VHH antibody and a Fc region of a rabbit antibody.

In any of the preceding embodiments, the isolated camelid antibody can be a humanized antibody.

In any of the preceding embodiments, the isolated camelid antibody can be conjugated to a detectable label. In certain aspects, the detectable label is a colorimetric, a radioactive, an enzymatic, a luminescent or a fluorescent label.

In any of the preceding embodiments, the detectable label can be a soluble label or a particle (such as a nanoparticle or a microparticle) or particulate label.

In any of the preceding embodiments, the isolated camelid antibody can be attached to a solid surface, such as a blot, a membrane, a sheet, a paper, a bead, a particle (such as a nanoparticle or a microparticle), an assay plate, an array, a glass slide, a microtiter, or an ELISA plate.

In one aspect, disclosed herein is a method for detecting an HCV core antigen polypeptide in a sample, which method comprises contacting the HCV core antigen polypeptide in the sample with an isolated camelid antibody of any of the preceding embodiments, and detecting a polypeptide-antibody complex formed between the HCV core antigen polypeptide in the sample and the isolated camelid antibody to assess the presence, absence and/or amount of the HCV core antigen polypeptide in the sample. In one aspect, the sample is from a subject, e.g., a mammal. In one embodiment, the mammal is a human. In any of the preceding embodiments, the sample can be a biological sample, such as a plasma sample, serum sample, dried blood spot, urine sample, tissue sample, and buccal swab.

In any of the preceding embodiments, the method can be used for diagnosis, prognosis, stratification, risk assessment, or treatment monitoring of an HCV infection.

In any of the preceding embodiments, the method can be used for identifying HCV infection in a seronegative mammal, identifying a seropositive mammal that is actively infected with HCV, or for monitoring an anti-HCV therapy.

In any of the preceding embodiments, the sample can be selected from the group consisting of a whole blood sample, a serum, a plasma, a urine and a saliva sample. In any of the preceding embodiments, the sample can be a clinical sample.

In any of the preceding embodiments, the polypeptide-antibody complex can be assessed by a sandwich or competitive assay format. In one aspect, the camelid antibody is attached to a surface and functions as a capture antibody. In one embodiment, the camelid antibody is labeled. In one aspect, the polypeptide-antibody complex is assessed by a sandwich assay format that uses two camelid antibodies, one being a capture antibody and the other being a labeled antibody. In any of the preceding embodiments, the antibody can be used in combination with antibodies from other species, such as human, mouse, rabbit, or goat, and the antibodies can be monoclonal or polyclonal. In any of the preceding embodiments, the antibody can be conjugated to a nano- or micro-particle.

In any of the preceding embodiments, the polypeptide-antibody complex can be assessed by a format selected from the group consisting of an enzyme-linked immunosorbent assay (ELISA), immunoblotting, immunoprecipitation, radioimmunoassay (RIA), immunostaining, latex agglutination, indirect hemagglutination assay (IHA), complement fixation, indirect immunofluorescent assay (IFA), nephelometry, flow cytometry assay, plasmon resonance assay, chemiluminescence assay, lateral flow immunoassay, μ-capture assay, inhibition assay and avidity assay.

In any of the preceding embodiments, the polypeptide-antibody complex can be assessed in a homogeneous or a heterogeneous assay format. In any of the preceding embodiments, the method can further comprise disassociating the HCV core antigen polypeptide in the sample from an antibody of the subject to be tested. In one aspect, the HCV core antigen polypeptide in the sample is disassociated from the antibody of the subject to be tested by changing the pH of the sample to be 4 or lower, or to be 9 or higher, by treating the sample with a protein denaturing agent, and/or by heating the sample to between about 35° C. and about 95° C., preferably to between about 45° C. and about 70° C., concurrently with or before contacting the sample with the camelid antibody. In some aspects, the protein denaturing agent is guanidine hydrochloride (e.g., about 1 M to about 6 M), guanidinium thiocyanate (e.g., about 1 M to about 6 M), SDS (e.g., about 0.1% to about 2%), β-mercaptoethanol, DTT or other reducing agent for disulfide bond disruption at various concentrations, or urea (e.g., about 2 M to about 8 M), or any combination thereof.

In any of the preceding embodiments, the method can further comprise adjusting the pH of the sample to between about 6 and about 8, and/or removing the protein denaturing agent concurrently with or before contacting the sample with the camelid antibody.

In any of the preceding embodiments, the camelid antibody can be a camelid VHH antibody, and the sample can be contacted with the camelid VHH antibody at a pH that is at 4 or lower, or at 9 or higher, and/or in the presence of the protein denaturing agent. In one aspect, the camelid VHH antibody is a llama VHH antibody.

In another aspect, provided herein is a kit for detecting an HCV core antigen polypeptide, which kit comprises, in a container, an isolated camelid antibody of any of the preceding embodiments.

In another aspect, provided herein is a lateral flow device comprising a matrix that comprises an isolated camelid antibody of any of the preceding embodiments immobilized on a test site on the matrix downstream from a sample application site on the matrix. In one aspect, the lateral flow device further comprises a labeled camelid antibody of any of the preceding embodiments on the matrix upstream from the test site, said labeled camelid antibody being capable of moved by a liquid sample and/or a further liquid to the test site and/or a control site to generate a detectable signal. The isolated camelid antibody disclosed herein is also compatible with other immunoassays such as ELISA.

In yet another aspect, provided herein is a method for detecting an HCV core antigen polypeptide in a liquid sample, which method comprises: a) contacting a liquid sample with the lateral flow device of any of the preceding embodiments, wherein the liquid sample is applied to a site of the lateral flow device upstream of the test site; b) transporting an HCV core antigen polypeptide, if present in the liquid sample, and a labeled camelid antibody of any of the preceding embodiments to the test site; and c) assessing the presence, absence, and/or amount of a signal generated by the labeled camelid antibody at the test site to determining the presence, absence and/or amount of the HCV core antigen polypeptide in the liquid sample.

In one aspect, provided herein is a method for detecting an analyte in a sample from a subject, which method comprises: a) disassociating an analyte in a sample from a subject that is bound to an antibody of the subject from the antibody of the subject by changing the pH of the sample to be 4 or lower, or to be 9 or higher, and/or by treating the sample with a protein denaturing agent, and/or by heating the sample to between about 35° C. and about 95° C., preferably to between about 45° C. and about 70° C.; b) contacting the analyte disassociated from the antibody of the subject with a camelid VHH antibody at a pH that is at 4 or lower, or at 9 or higher, and/or in the presence of the protein denaturing agent, and/or at a temperature of about 35° C. and about 95° C., preferably about 45° C. and about 70° C., and detecting an analyte-antibody complex formed between the disassociated analyte and the camelid antibody to assess the presence, absence and/or amount of the analyte in the sample.

In one embodiment, the analyte is selected from the group consisting of a cell, a cellular organelle, a virus, a molecule and an aggregate or complex thereof. In one aspect, the cell is selected from the group consisting of an animal cell, a plant cell, a fungus cell, a bacterium cell, a recombinant cell and a cultured cell. In another aspect, the cellular organelle is selected from the group consisting of a nucleus, a mitochondrion, a chloroplast, a ribosome, an ER, a Golgi apparatus, a lysosome, a proteasome, a secretory vesicle, a vacuole and a microsome. In yet another aspect, the molecule is selected from the group consisting of an inorganic molecule, an organic molecule and a complex thereof. In still another aspect, the inorganic molecule is an ion selected from the group consisting of a sodium, a potassium, a magnesium, a calcium, a chlorine, an iron, a copper, a zinc, a manganese, a cobalt, an iodine, a molybdenum, a vanadium, a nickel, a chromium, a fluorine, a silicon, a tin, a boron and an arsenic ion. In one embodiment, the organic molecule is selected from the group consisting of an amino acid, a peptide, a protein, a polypeptide, a nucleoside, a nucleotide, an oligonucleotide, a nucleic acid, a polynucleotide, a vitamin, a monosaccharide, an oligosaccharide, a polysaccharide a carbohydrate, a lipid and a complex thereof.

In any of the preceding embodiments, the analyte can be a marker for a disease, disorder or infection. In any of the preceding embodiments, the method can be used for diagnosis, prognosis, stratification, risk assessment, and/or treatment monitoring of the disease, disorder or infection. In any of the preceding embodiments, the analyte can be a marker for is bacterial or viral infection. In any of the preceding embodiments, the analyte can be a marker for HCV infection. In any of the preceding embodiments, the analyte can be an HCV polypeptide. In any of the preceding embodiments, the HCV polypeptide can be an HCV core antigen polypeptide.

In any of the preceding embodiments, the method can be used for diagnosis, prognosis, stratification, risk assessment, and/or treatment monitoring of an HCV infection.

In any of the preceding embodiments, the method can be used for identifying HCV infection in a seronegative mammal, identifying a seropositive mammal that is actively infected with HCV, and/or for monitoring an anti-HCV therapy. In any of the preceding embodiments, the subject can be a mammal. In one embodiment, the mammal is a human.

In any of the preceding embodiments, the sample can be selected from the group consisting of a whole blood sample, a serum, a plasma, a urine and a saliva sample. In any of the preceding embodiments, the sample can be a clinical sample.

In any of the preceding embodiments, the analyte can be disassociated from the antibody of the subject by changing the pH of the sample to be 4 or lower. In any of the preceding embodiments, the analyte can be disassociated from the antibody of the subject by changing the pH of the sample to be 9 or higher. In any of the preceding embodiments, the analyte can be disassociated from the antibody of the subject by heating the sample to between about 35° C. and about 95° C., preferably to between about 45° C. and about 70° C.

In any of the preceding embodiments, the analyte can be disassociated from the antibody of the subject by treating the sample with a protein denaturing agent. In certain embodiments, the protein denaturing agent is guanidine hydrochloride (e.g., about 1 M to about 6 M), guanidinium thiocyanate (e.g., about 1 M to about 6 M), SDS (e.g., about 0.1% to about 2%), 0-mercaptoethanol, DTT or other reducing agent for disulfide bond disruption at various concentrations, or urea (e.g., about 2 M to about 8 M), or any combination thereof.

In any of the preceding embodiments, the analyte can be disassociated from the antibody of the subject by changing the pH of the sample to be 4 or lower, by treating the sample with a protein denaturing agent, and/or by heating the sample to between about 35° C. and about 95° C., preferably to between about 45° C. and about 70° C.

In any of the preceding embodiments, the analyte can be disassociated from the antibody of the subject by changing the pH of the sample to be 9 or higher, by treating the sample with a protein denaturing agent, and/or by heating the sample to between about 35° C. and about 95° C., preferably to between about 45° C. and about 70° C.

In any of the preceding embodiments, the analyte disassociated from the antibody of the subject can be contacted with the camelid VHH antibody at a pH that is at 4 or lower. In any of the preceding embodiments, the analyte disassociated from the antibody of the subject can be contacted with the camelid VHH antibody at pH that is at 9 or higher. In any of the preceding embodiments, the analyte disassociated from the antibody of the subject can be contacted with the camelid VHH antibody at a temperature of about 35° C. and about 95° C., preferably about 45° C. and about 70° C. In any of the preceding embodiments, the analyte disassociated from the antibody of the subject can be contacted with the camelid VHH antibody in the presence of the protein denaturing agent. In one aspect, the protein denaturing agent is guanidine hydrochloride (e.g., about 1 M to about 6 M), guanidinium thiocyanate (e.g., about 1 M to about 6 M), SDS (e.g., about 0.1% to about 2%), 0-mercaptoethanol, DTT or other reducing agent for disulfide bond disruption at various concentrations, or urea (e.g., about 2 M to about 8 M), or any combination thereof.

In any of the preceding embodiments, the analyte disassociated from the antibody of the subject can be contacted with the camelid VHH antibody at a pH that is at 4 or lower and in the presence of the protein denaturing agent. In any of the preceding embodiments, the analyte disassociated from the antibody of the subject can be contacted with the camelid VHH antibody at pH that is at 9 or higher and in the presence of the protein denaturing agent.

In any of the preceding embodiments, the camelid VHH antibody can be a llama VHH antibody. In one aspect, the llama VHH antibody is a fusion llama VHH antibody that comprises a variable region of the llama VHH antibody and a constant region of a rabbit antibody.

In any of the preceding embodiments, the analyte-antibody complex can be assessed by a sandwich or competitive assay format. In one aspect, the camelid antibody is attached to a surface and functions as a capture antibody. In another aspect, the camelid antibody is labeled. In some embodiments, the analyte-antibody complex is assessed by a sandwich assay format that uses two camelid antibodies, one being a capture antibody and the other being a labeled antibody.

In any of the preceding embodiments, the analyte-antibody complex can be assessed by a format selected from the group consisting of an enzyme-linked immunosorbent assay (ELISA), immunoblotting, immunoprecipitation, radioimmunoassay (RIA), immunostaining, latex agglutination, indirect hemagglutination assay (IHA), complement fixation, indirect immunofluorescent assay (IFA), nephelometry, flow cytometry assay, plasmon resonance assay, chemiluminescence assay, lateral flow immunoassay, μ-capture assay, inhibition assay and avidity assay.

In any of the preceding embodiments, the analyte-antibody complex can be assessed in a homogeneous or a heterogeneous assay format. In some aspects, the analyte-antibody complex is assessed by a lateral flow sandwich assay format that uses two camelid antibodies, one being a capture antibody and the other being a labeled antibody. In one embodiment, the labeled antibody is labeled with a particle (such as a nanoparticle or a microparticle) or particulate label.

In any of the preceding embodiments, the steps a) and b) can be conducted concurrently.

In any of the preceding embodiments, the step a) can be conducted before the step b).

In any of the preceding embodiments, the method can be conducted to assess the presence or absence of the analyte in the sample. In any of the preceding embodiments, the method can be conducted to assess the amount of the analyte in the sample.

### BRIEF DESCRIPTION OF THE DRAWINGS

FIG. 1 shows serum ELISA titers for various antigens according to certain aspects of the present disclosure. Test bleeds were collected on the days indicated. Serum was serial diluted by a factor of 10.

FIG. 2 shows coomassie blue stained SDS-PAGE gel (4-20% gradient gel). The left four lanes were loaded with 3-5pg of purified VHH from different clones, the right lane was loaded with NEB pre-stained color plus protein marker. The 23 kDa maker is indicated.

FIG. 3A shows the results of direct ELISA of VHH antibodies. FIG. 3B shows the results of competition/Inhibition ELISA of VHH antibodies.

FIG. 4 shows a competition lateral flow immunoassay using VHH-rFc fusion antibody for AG01.

FIG. 5 shows the results of a lateral flow assays with guanidine hydrochloride and SDS in the sample buffer.

FIG. 6A shows an illustration of a test strip and optical read-out. FIG. 6B shows a portable QD reader.

FIG. 7 shows a chart for ELISA results at 1:1000 of serum dilution, showing first positive titer.

FIG. 8 shows the anti-sera titer for the llama (left) and the rabbit (right), showing ELISA results at 1:10,000 dilution.

FIG. 9A shows the total RNA isolated from PBMC cells. FIG. 9B shows PCR product for VH and VHH. FIG. 9C shows DNA prior to library ligation, vector (pADL20c, digested with BglI), insert, digested with sfiI.

FIGS. 10A and 10B show affinity and specificity of the purified antibodies.

FIG. 11 shows binding epitope of the monoclonal antibody C7-50.

FIG. 12 shows sandwich ELISA results showing that monoclonal antibody C7-50 is able to pair with anti-serum from either the llama or the rabbit.

FIG. 13 shows LFIA results for detecting 1-gal-192 core with antibody pair of mAb C7-50 and the llama anti-serum.

FIG. 14 shows a lateral flow assay testing a labeled antibody according to one aspect of the present disclosure.

### DETAILED DESCRIPTION

A detailed description of one or more embodiments of the claimed subject matter is provided below along with accompanying figures that illustrate the principles of the claimed subject matter. The claimed subject matter is described in connection with such embodiments, but is not limited to any particular embodiment. It is to be understood that the claimed subject matter may be embodied in various forms, and encompasses numerous alternatives, modifications and equivalents. Therefore, specific details disclosed herein are not to be interpreted as limiting, but rather as a basis for the claims and as a representative basis for teaching one skilled in the art to employ the claimed subject matter in virtually any appropriately detailed system, structure, or manner. Numerous specific details are set forth in the following description in order to provide a thorough understanding of the present disclosure. These details are provided for the purpose of example and the claimed subject matter may be practiced according to the claims without some or all of these specific details. It is to be understood that other embodiments can be used and structural changes can be made without departing from the scope of the claimed subject matter. It should be understood that the various features and functionality described in one or more of the individual embodiments are not limited in their applicability to the particular embodiment with which they are described. They instead can, be applied, alone or in some combination, to one or more of the other embodiments of the disclosure, whether or not such embodiments are described, and whether or not such features are presented as being a part of a described embodiment. For the purpose of clarity, technical material that is known in the technical fields related to the claimed subject matter has not been described in detail so that the claimed subject matter is not unnecessarily obscured.

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art. Many of the techniques and procedures described or referenced herein are well understood and commonly employed using conventional methodology by those skilled in the art.

All publications, including patent documents, scientific articles and databases, referred to in this application are incorporated by reference in their entireties for all purposes to the same extent as if each individual publication were individually incorporated by reference. If a definition set forth herein is contrary to or otherwise inconsistent with a definition set forth in the patents, patent applications, published applications or other publications that are herein incorporated by reference, the definition set forth herein prevails over the definition that is incorporated herein by reference. Citation of the publications or documents is not intended as an admission that any of them is pertinent prior art, nor does it constitute any admission as to the contents or date of these publications or documents.

All headings are for the convenience of the reader and should not be used to limit the meaning of the text that follows the heading, unless so specified.

The practice of the provided embodiments will employ, unless otherwise indicated, conventional techniques and descriptions of organic chemistry, polymer technology, molecular biology (including recombinant techniques), cell biology, biochemistry, and sequencing technology, which are within the skill of those who practice in the art. Such conventional techniques include polypeptide and protein synthesis and modification, polynucleotide synthesis and modification, polymer array synthesis, hybridization and ligation of polynucleotides, and detection of hybridization using a label. Specific illustrations of suitable techniques can be had by reference to the examples herein. However, other equivalent conventional procedures can, of course, also be used. Such conventional techniques and descriptions can be found in standard laboratory manuals such as Green, et al., Eds., Genome Analysis: A Laboratory Manual Series (Vols. I-IV) (1999); Weiner, Gabriel, Stephens, Eds., Genetic Variation: A Laboratory Manual (2007); Dieffenbach, Dveksler, Eds., PCR Primer: A Laboratory Manual (2003); Bowtell and Sambrook, DNA Microarrays: A Molecular Cloning Manual (2003); Mount, Bioinformatics: Sequence and Genome Analysis (2004); Sambrook and Russell, Condensed Protocols from Molecular Cloning: A Laboratory Manual (2006); and Sambrook and Russell, Molecular Cloning: A Laboratory Manual (2002) (all from Cold Spring Harbor Laboratory Press); Ausubel et al. eds., Current Protocols in Molecular Biology (1987); T. Brown ed., Essential Molecular Biology (1991), IRL Press; Goeddel ed., Gene Expression Technology (1991), Academic Press; A. Bothwell et al. eds., Methods for Cloning and Analysis of Eukaryotic Genes (1990), Bartlett Publ.; M. Kriegler, Gene Transfer and Expression (1990), Stockton Press; R. Wu et al. eds., Recombinant DNA Methodology (1989), Academic Press; M. McPherson et al., PCR: A Practical Approach (1991), IRL Press at Oxford University Press; Stryer, Biochemistry (4th Ed.) (1995), W. H. Freeman, New York N.Y.; Gait, Oligonucleotide Synthesis: A Practical Approach (2002), IRL Press, London; Nelson and Cox, Lehninger, Principles of Biochemistry (2000) 3rd Ed., W. H. Freeman Pub., New York, N.Y.; Berg, et al., Biochemistry (2002) 5th Ed., W. H. Freeman Pub., New York, N.Y.; D. Weir & C. Blackwell, eds., Handbook of Experimental Immunology (1996), Wiley-Blackwell; Cellular and Molecular Immunology (A. Abbas et al., W.B. Saunders Co. 1991, 1994); Current Protocols in Immunology (J. Coligan et al. eds. 1991), all of which are herein incorporated in their entireties by reference for all purposes.

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be included in the smaller ranges, and are also encompassed within the claimed subject matter, subject to any specifically excluded limit in the stated range. Where the stated range includes one or both of the limits, ranges excluding either or both of those included limits are also included in the claimed subject matter. This applies regardless of the breadth of the range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6.

### I. Definitions

As used herein, the singular forms “a,” “an,” and “the” include plural referents unless the context clearly dictates otherwise. For example, “a” or “an” means “at least one” or “one or more.” It is understood that aspects and variations described herein include “consisting” and/or “consisting essentially of” aspects and variations.

The term “about” as used herein refers to the usual error range for the respective value readily known to the skilled person in this technical field. Reference to “about” a value or parameter herein includes (and describes) embodiments that are directed to that value or parameter per se. For example, description referring to “about X” includes description of “X”.

As used herein, a composition refers to any mixture of two or more products, substances, or compounds, including cells. It may be a solution, a suspension, liquid, powder, a paste, aqueous, non-aqueous or any combination thereof.

The term “antibody” herein is used in the broadest sense and includes polyclonal and monoclonal antibodies, including intact antibodies and functional (antigen-binding) antibody fragments, including fragment antigen binding (Fab) fragments, F(ab′)₂ fragments, Fab′ fragments, Fv fragments, recombinant IgG (rIgG) fragments, single chain antibody fragments, including single chain variable fragments (scFv), and single domain antibodies (e.g., sdAb, sdFv, nanobody) fragments. The term encompasses genetically engineered and/or otherwise modified forms of immunoglobulins, such as intrabodies, peptibodies, chimeric antibodies, fully human antibodies, humanized antibodies, and heteroconjugate antibodies, multispecific, e.g., bispecific, antibodies, diabodies, triabodies, and tetrabodies, tandem di-scFv, tandem tri-scFv. Unless otherwise stated, the term “antibody” should be understood to encompass functional antibody fragments thereof. The term also encompasses intact or full-length antibodies, including antibodies of any class or sub-class, including IgG and sub-classes thereof, IgM, IgE, IgA, and IgD.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG₁, IgG₂, IgG₃, IgG₄, IgA₁, and IgA₂. The heavy chain constant domains that correspond to the different classes of immunoglobulins are called α, δ, ε, γ, and μ, respectively.

The terms “complementarity determining region,” and “CDR,” synonymous with “hypervariable region” or “HVR,” are known in the art to refer to non-contiguous sequences of amino acids within antibody variable regions, which confer antigen specificity and/or binding affinity. In general, there are three CDRs in each heavy chain variable region (CDR-H1, CDR-H2, CDR-H3) and three CDRs in each light chain variable region (CDR-L1, CDR-L2, CDR-L3). “Framework regions” and “FR” are known in the art to refer to the non-CDR portions of the variable regions of the heavy and light chains. In general, there are four FRs in each full-length heavy chain variable region (FR-H1, FR-H2, FR-H3, and FR-H4), and four FRs in each full-length light chain variable region (FR-L1, FR-L2, FR-L3, and FR-LA).

The precise amino acid sequence boundaries of a given CDR or FR can be readily determined using any of a number of well-known schemes, including those described by Kabat et al. (1991), “Sequences of Proteins of Immunological Interest,” 5th Ed. Public Health Service, National Institutes of Health, Bethesda, Md. (“Kabat” numbering scheme), Al-Lazikani et al., (1997) JMB 273,927-948 (“Chothia” numbering scheme), MacCallum et al., J. Mol. Biol. 262:732-745 (1996), “Antibody-antigen interactions: Contact analysis and binding site topography,” J. Mol. Biol. 262, 732-745.” (“Contact” numbering scheme), Lefranc M P et al., “IMGT unique numbering for immunoglobulin and T cell receptor variable domains and Ig superfamily V-like domains,” Dev Comp Immunol, 2003 January; 27(1):55-77 (“IMGT” numbering scheme), and Honegger A and Plückthun A, “Yet another numbering scheme for immunoglobulin variable domains: an automatic modeling and analysis tool,” J Mol Biol, 2001 Jun. 8; 309(3):657-70, (“Aho” numbering scheme).

The boundaries of a given CDR or FR may vary depending on the scheme used for identification. For example, the Kabat scheme is based structural alignments, while the Chothia scheme is based on structural information. Numbering for both the Kabat and Chothia schemes is based upon the most common antibody region sequence lengths, with insertions accommodated by insertion letters, for example, “30a,” and deletions appearing in some antibodies. The two schemes place certain insertions and deletions (“indels”) at different positions, resulting in differential numbering. The Contact scheme is based on analysis of complex crystal structures and is similar in many respects to the Chothia numbering scheme.

Thus, unless otherwise specified, a “CDR” or “complementary determining region,” or individual specified CDRs (e.g., “CDR-H1, CDR-H2), of a given antibody or region thereof, such as a variable region thereof, should be understood to encompass a (or the specific) complementary determining region as defined by any of the aforementioned schemes. For example, where it is stated that a particular CDR (e.g., a CDR-H3) contains the amino acid sequence of a corresponding CDR in a given V_{H} or VL amino acid sequence, it is understood that such a CDR has a sequence of the corresponding CDR (e.g., CDR-H3) within the variable region, as defined by any of the aforementioned schemes.

Likewise, unless otherwise specified, a FR or individual specified FR(s) (e.g., FR-H1, FR-H2), of a given antibody or region thereof, such as a variable region thereof, should be understood to encompass a (or the specific) framework region as defined by any of the known schemes. In some instances, the scheme for identification of a particular CDR, FR, or FRs or CDRs is specified, such as the CDR as defined by the Kabat, Chothia, or Contact method.

The term “variable region” or “variable domain” refers to the domain of an antibody heavy or light chain that is involved in binding the antibody to antigen. The variable domains of the heavy chain and light chain (V_{H} and V_{L}, respectively) of a native antibody generally have similar structures, with each domain comprising four conserved framework regions (FRs) and three CDRs. See, e.g., Kindt et al., Kuby Immunology, 6th ed., W.H. Freeman and Co., page 91 (2007). A single V_{H} or V_{L} domain may be sufficient to confer antigen-binding specificity. Furthermore, antibodies that bind a particular antigen may be isolated using a V_{H} or V_{L} domain from an antibody that binds the antigen to screen a library of complementary V_{L} or V_{H} domains, respectively. See, e.g., Portolano et al., J. Immunol. 150:880-887 (1993); Clarkson et al., Nature 352:624-628 (1991).

The term “Fc region” herein is used to define a C-terminal region of an immunoglobulin heavy chain that contains at least a portion of the constant region. The term includes native sequence Fc regions and variant Fc regions. In one embodiment, a human IgG heavy chain Fc region extends from Cys226, or from Pro230, to the carboxyl-terminus of the heavy chain. However, the C-terminal lysine (Lys447) of the Fc region may or may not be present. Unless otherwise specified herein, numbering of amino acid residues in the Fc region or constant region is according to the EU numbering system, also called the EU index, as described in Kabat el al., Sequences of Proteins of Immunological Interest, 5th Ed. Public Health Service, National Institutes of Health, Bethesda, Md., 1991.

Among the provided antibodies are antibody fragments. An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies; linear antibodies; single-chain antibody molecules (e.g. scFv); and multispecific antibodies formed from antibody fragments. In particular embodiments, the antibodies are single-chain antibody fragments comprising a variable heavy chain region and/or a variable light chain region, such as scFvs.

Single-domain antibodies are antibody fragments comprising all or a portion of the heavy chain variable domain or all or a portion of the light chain variable domain of an antibody. In certain embodiments, a single-domain antibody is a camelid single-domain antibody.

Antibody fragments can be made by various techniques, including but not limited to proteolytic digestion of an intact antibody as well as production by recombinant host cells. In some embodiments, the antibodies are recombinantly-produced fragments, such as fragments comprising arrangements that do not occur naturally, such as those with two or more antibody regions or chains joined by synthetic linkers, e.g., peptide linkers, and/or that are may not be produced by enzyme digestion of a naturally-occurring intact antibody.

A “humanized” antibody is an antibody in which all or substantially all CDR amino acid residues are derived from non-human CDRs and all or substantially all FR amino acid residues are derived from human FRs. The term “chimeric” antibody refers to an antibody in which a portion of the heavy and/or light chain is derived from a particular source or species, while the remainder of the heavy and/or light chain is derived from a different source or species.

Among the provided antibodies are monoclonal antibodies, including monoclonal antibody fragments. The term “monoclonal antibody” as used herein refers to an antibody obtained from or within a population of substantially homogeneous antibodies, i.e., the individual antibodies comprising the population are identical, except for possible variants containing naturally occurring mutations or arising during production of a monoclonal antibody preparation, such variants generally being present in minor amounts. In contrast to polyclonal antibody preparations, which typically include different antibodies directed against different epitopes, each monoclonal antibody of a monoclonal antibody preparation is directed against a single epitope on an antigen. The term is not to be construed as requiring production of the antibody by any particular method. A monoclonal antibody may be made by a variety of techniques, including but not limited to generation from a hybridoma, recombinant DNA methods, phage-display and other antibody display methods.

The terms “polypeptide” and “protein” are used interchangeably to refer to a polymer of amino acid residues, and are not limited to a minimum length. Polypeptides, including the provided antibodies and antibody chains and other peptides, e.g., linkers, the HCV core antigen polypeptides, and/or the anti-HCV core antigen antibodies, may include amino acid residues including natural and/or non-natural amino acid residues. The terms also include post-expression modifications of the polypeptide, for example, glycosylation, sialylation, acetylation, phosphorylation, and the like. In some aspects, the polypeptides may contain modifications with respect to a native or natural sequence, as long as the protein maintains the desired activity. These modifications may be deliberate, as through site-directed mutagenesis, or may be accidental, such as through mutations of hosts which produce the proteins or errors due to PCR amplification.

“Affinity” refers to the strength of the sum total of non-covalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (Kd). Affinity can be measured by common methods known in the art, including those described herein. Specific illustrative and exemplary embodiments for measuring binding affinity are described in the following

An “affinity matured” antibody refers to an antibody with one or more alterations in one or more hypervariable regions (HVRs), compared to a parent antibody which does not possess such alterations, such alterations resulting in an improvement in the affinity of the antibody for antigen.

The term “HCV core antigen” as used herein encompasses “full-length,” unprocessed HCV core antigen as well as any form of HCV core antigen that results from processing in the cell or in vitro, or any mutation in the cell or in vitro. The term also encompasses naturally occurring variants of HCV core antigen, e.g., splice variants or allelic variants.

The terms “anti-HCV core antigen antibody” and “an antibody that binds to HCV core antigen” refer to an antibody that is capable of binding HCV core antigen with sufficient affinity and/or specificity. In some embodiments, such an antibody is useful as a diagnostic and/or therapeutic agent in targeting HCV core antigen. In one embodiment, the extent of binding of an anti-HCV core antigen antibody to an unrelated, non-HCV core antigen protein or peptide is less than about 10% of the binding of the antibody to HCV core antigen as measured, e.g., by a radioimmunoassay (RIA). In certain embodiments, an antibody that binds to HCV core antigen has a dissociation constant (Kd) of ≤1 μM, ≤100 nM, ≤10 nM, ≤1 nM, ≤0.1 nM, ≤0.01 nM, or ≤0.001 nM (e.g., 10⁻⁸ M or less, from 10⁻⁸ M to 10⁻¹³ M, or from 10⁻⁹ M to 10⁻¹³ M). In certain embodiments, an anti-HCV core antigen antibody binds to an epitope of HCV core antigen that is conserved among HCV core antigen from different HCV variants.

As used herein, the term “specific binding” refers to the specificity of a binder, e.g., an antibody, such that it preferentially binds to a target, such as a polypeptide antigen. When referring to a binding partner, e.g., protein, nucleic acid, antibody or other affinity capture agent, etc., “specific binding” can include a binding reaction of two or more binding partners with high affinity and/or complementarity to ensure selective hybridization under designated assay conditions. Typically, specific binding will be at least three times the standard deviation of the background signal. Thus, under designated conditions the binding partner binds to its particular target molecule and does not bind in a significant amount to other molecules present in the sample. Recognition by a binder or an antibody of a particular target in the presence of other potential interfering substances is one characteristic of such binding. Preferably, binders, antibodies or antibody fragments that are specific for or bind specifically to a target bind to the target with higher affinity than binding to other non-target substances. Also preferably, binders, antibodies or antibody fragments that are specific for or bind specifically to a target avoid binding to a significant percentage of non-target substances, e.g., non-target substances present in a testing sample. In some embodiments, binders, antibodies or antibody fragments of the present disclosure avoid binding greater than about 90% of non-target substances, although higher percentages are clearly contemplated and preferred. For example, binders, antibodies or antibody fragments of the present disclosure avoid binding about 91%, about 92%, about 93%, about 94%, about 95%, about 96%, about 97%, about 98%, about 99%, and about 99% or more of non-target substances. In other embodiments, binders, antibodies or antibody fragments of the present disclosure avoid binding greater than about 10%, 20%, 30%, 40%, 50%, 60%, or 70%, or greater than about 75%, or greater than about 80%, or greater than about 85% of non-target substances.

The term “cytotoxic agent” as used herein refers to a substance that inhibits or prevents a cellular function and/or causes cell death or destruction. Cytotoxic agents include, but are not limited to, radioactive isotopes (e.g., At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu); chemotherapeutic agents or drugs (e.g., methotrexate, adriamicin, vinca alkaloids (vincristine, vinblastine, etoposide), doxorubicin, melphalan, mitomycin C, chlorambucil, daunorubicin or other intercalating agents); growth inhibitory agents; enzymes and fragments thereof such as nucleolytic enzymes; antibiotics; toxins such as small molecule toxins or enzymatically active toxins of bacterial, fungal, plant or animal origin, including fragments and/or variants thereof; and the various antitumor or anticancer agents disclosed below.

“Effector functions” refer to those biological activities attributable to the Fc region of an antibody, which vary with the antibody isotype. Examples of antibody effector functions include: C1q binding and complement dependent cytotoxicity (CDC); Fc receptor binding; antibody-dependent cell-mediated cytotoxicity (ADCC); phagocytosis; down regulation of cell surface receptors (e.g. B cell receptor); and B cell activation.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

An “immunoconjugate” is an antibody conjugated to one or more heterologous molecule(s), including but not limited to a cytotoxic agent. In some embodiments, the antibody is or is part of an immunoconjugate, in which the antibody is conjugated to one or more heterologous molecule(s), such as, but not limited to, a cytotoxic or an imaging agent. Cytotoxic agents include, but are not limited to, radioactive isotopes (e.g., exemplary radioactive isotopes include At²¹, I¹³¹, I¹²⁵, Y⁹⁰, Re¹⁸⁶, Re¹⁸⁸, Sm¹⁵³, Bi²¹², P³², Pb²¹² and radioactive isotopes of Lu); chemotherapeutic agents (e.g., methotrexate, adriamicin, vinca alkaloids (vincristine, vinblastine, etoposide), doxorubicin, melphalan, mitomycin C, chlorambucil, daunorubicin or other intercalating agents); growth inhibitory agents; enzymes and fragments thereof such as nucleolytic enzymes; antibiotics; toxins such as small molecule toxins or enzymatically active toxins. In some embodiments, the antibody is conjugated to one or more cytotoxic agents, such as chemotherapeutic agents or drugs, growth inhibitory agents, toxins (e.g., protein toxins, enzymatically active toxins of bacterial, fungal, plant, or animal origin, or fragments thereof), or radioactive isotopes.

Among the immunoconjugates are antibody-drug conjugates (ADCs), in which an antibody is conjugated to one or more drugs, including but not limited to a maytansinoid (see U.S. Pat. Nos. 5,208,020, 5,416,064 and European Patent EP 0 425 235 BI); an auristatin such as monomethylauristatin drug moieties DE and DF (MMAE and MMAF) (see U.S. Pat. Nos. 5,635,483 and 5,780,588, and 7,498,298); a dolastatin; a calicheamicin or derivative thereof (see U.S. Pat. Nos. 5,712,374, 5,714,586, 5,739,116, 5,767,285, 5,770,701, 5,770,710, 5,773,001, and 5,877,296; Hinman et al., Cancer Res. 53:3336-3342 (1993); and Lode et al., Cancer Res. 58:2925-2928 (1998)); an anthracycline such as daunomycin or doxorubicin (see Kratz et al., Current Med. Chem. 13:477-523 (2006); Jeffrey et al., Bioorganic & Med. Chem. Letters 16:358-362 (2006); Torgov et al., Bioconj. Chem. 16:717-721 (2005); Nagy et al., Proc. Natl. Acad. Sci. USA 97:829-834 (2000); Dubowchik et al., Bioorg. & Med. Chem. Letters 12:1529-1532 (2002); King et al., J. Med. Chem. 45:4336-4343 (2002); and U.S. Pat. No. 6,630,579); methotrexate; vindesine; a taxane such as docetaxel, paclitaxel, larotaxel, tesetaxel, and ortataxel; a trichothecene; and CC1065.

Conjugates of an antibody and cytotoxic agent may be made using any of a number of known protein coupling agents, e.g., linkers, (see Vitetta et al., Science 238:1098 (1987)), WO94/11026. The linker may be a “cleavable linker” facilitating release of a cytotoxic drug in the cell, such as acid-labile linkers, peptidase-sensitive linkers, photolabile linkers, dimethyl linkers, and disulfide-containing linkers (Chari et al., Cancer Res. 52:127-131 (1992); U.S. Pat. No. 5,208,020).

An “individual” or “subject” includes a mammal. Mammals include, but are not limited to, domesticated animals (e.g., cows, sheep, cats, dogs, and horses), primates (e.g., humans and non-human primates such as monkeys), rabbits, and rodents (e.g., mice and rats). An “individual” or “subject” may include birds such as chickens, vertebrates such as fish and mammals such as mice, rats, rabbits, cats, dogs, pigs, cows, ox, sheep, goats, horses, monkeys and other non-human primates. In certain embodiments, the individual or subject is a human.

As used herein, a “sample” can be a solution, a suspension, liquid, powder, a paste, aqueous, non-aqueous or any combination thereof.

In some embodiments, the sample is a biological sample. A biological sample of the present disclosure encompasses a sample in the form of a solution, a suspension, a liquid, a powder, a paste, an aqueous sample, or a non-aqueous sample. As used herein, a “biological sample” includes any sample obtained from a living or viral (or prion) source or other source of macromolecules and biomolecules, and includes any cell type or tissue of a subject from which nucleic acid, protein and/or other macromolecule can be obtained. The biological sample can be a sample obtained directly from a biological source or a sample that is processed. For example, isolated nucleic acids that are amplified constitute a biological sample. Biological samples include, but are not limited to, body fluids, such as blood, plasma, serum, cerebrospinal fluid, synovial fluid, urine and sweat, tissue and organ samples from animals and plants and processed samples derived therefrom. In some embodiments, the sample can be derived from a tissue or a body fluid, for example, a connective, epithelium, muscle or nerve tissue; a tissue selected from the group consisting of brain, lung, liver, spleen, bone marrow, thymus, heart, lymph, blood, bone, cartilage, pancreas, kidney, gall bladder, stomach, intestine, testis, ovary, uterus, rectum, nervous system, gland, and internal blood vessels; or a body fluid selected from the group consisting of blood, urine, saliva, bone marrow, sperm, an ascitic fluid, and subfractions thereof, e.g., serum or plasma.

An “isolated” antibody is one which has been separated from a component of its natural environment. In some embodiments, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC). For review of methods for assessment of antibody purity, see, e.g., Flatman et al., J. Chromatogr. B 848:79-87 (2007).

An “isolated” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared.

### II. HCV Core Antigen Polypeptides

Currently there are two serological methods approved by the US FDA for the laboratory diagnosis and monitoring of HCV infection: HCV antibody detection and HCV RNA detection. Both methods have their limitations.

The presence of anti-HCV antibodies typically cannot be confirmed until 12-27 weeks after exposure, creating a window period of sero-negativity and potential infectivity. See Centers for Disease Control and Prevention, Recommendations for prevention and control of hepatitis C virus (HCV) infection and HCV-related chronic disease, MMWR Recomm Rep, 1998, 47(RR-19):1-39. HCV RNA detected by polymerase chain reaction (PCR) becomes positive within days of infection, RNA-PCR has become the method of choice for early diagnosis. See Fang et al., Fluctuation of HCV viral load before sero-conversion in a healthy volunteer blood donor, Transfusion, 2003, 43(4):541-4; Lee et al., Efficacy of a hepatitis C virus core antigen enzyme-linked immunosorbent assay for the identification of “window-phase” blood donations, Vox Sang, 2001, 80(1): 19-23. However, the tests have to be done in a laboratory following strict guidelines due to the nature of RNA (easy to degrade) and PCR (amplification and cross-contamination to cause false positive).

Immunoassays for detection of hepatitis C core antigen are being used in Europe and Asia as another early diagnosis tool. However, these immunoassays all require to be conducted in a laboratory with complicated procedures to process the samples prior to the immunoassays.

Therefore, there is an urgent need for a rapid test that is suitable for point-of-care (POC) use and early detection of HCV infection. In some aspects, provided herein is a technology platform for isolating highly specific single domain antibodies from immunized llamas and use of these antibodies in lateral flow immunoassays (LFIA). In other aspects, this technology is used to develop HCV core antigen specific antibodies and to produce LFIA devices for rapid and early diagnosis of HCV.

In one aspect, HCV core antigen is used as a sero-marker for early detection of HCV infection. HCV contains a single-stranded, positive-sense RNA molecule of 9.6 kb with one long open reading frame coding for a large polyprotein of about 3000 amino acids which undergoes co-translational and post-translational cleavage by host and viral proteases to yield individual viral proteins. See Choo et al., Isolation of a cDNA clone derived from a blood-borne non-A, non-B viral hepatitis genome, Science, 1989, 244(4902):359-62. HCV undergoes rapid mutation in a hypervariable region of the genome coding for the envelope proteins and escapes immune surveillance by the host. As a consequence, most HCV-infected people develop chronic infection. See Centers for Disease Control and Prevention, Recommendations for prevention and control of hepatitis C virus (HCV) infection and HCV-related chronic disease, MMWR Recomm Rep, 1998, 47(RR-19):1-39.

The N-terminal quarter of the HCV genome encodes the core and structural proteins. These consist of a non-glycosylated nucleic acid-binding nucleocapsid protein (C) of about 190 amino acids (about 21 kD, HCV core antigen) and one or possibly two membrane-associated glycoproteins (E1 and E2/NS1) of about 190 and 370 amino acids respectively (33 and 70 kD when glycosylated). See Laufer et al., Saliva and serum as diagnostic media for antibody to hepatitis A virus in adults and in individuals who have received an inactivated hepatitis A vaccine, Clin Infect Dis, 1995, 20(4):868-71; Mandell et al., ed. Principle and Practice of Infectious Disease, 4th ed., Hepatitis C virus, 1995, Churchill Livingstone: New York, 1474-1486.

Detection and quantification of HCV core antigen by monoclonal antibodies directed against the conserved epitopes of the virus core protein have been described. Studies have revealed significant correlations of core antigen levels with those results from HCV-RNA assays, and HCV core antigen level has been suggested as a potential marker for viral replication. See Ergunay et al., Utility of a commercial quantitative hepatitis C virus core antigen assay in a diagnostic laboratory setting, Diagn Microbiol Infect Dis, 2011, 70(4):486-91.

Abbott Labs has developed an immunoassay system for clinically testing HCV core antigen (not available in the US). HCV Core Antigen testing can be utilized to identify HCV infection in seronegative individuals (pre-seroconversion window period detection), identify seropositive individuals who are actively infected with HCV, and as a complementary test to HCV NAT to monitor antiviral therapy. Unfortunately, due to the complication of host antibodies (sero-conversion), the samples need to be pretreated to dissociate antibody-bound antigen, lyse viral particles and expose core antigen, and inactivate the antibody. These complicated sample processing steps prior to the immunoassay are not suitable for rapid tests.

In some aspects, provided herein are llama single domain antibodies specific to HCV core antigen, for examples, antibodies that specifically bind to at least one epitope on one or more HCV core antigen polypeptides. In some aspects, these antibodies are resistant to denaturant and can overcome the above mentioned hurdles associated with existing methods. In other aspects, these antibodies are used to develop rapid tests for HCV core antigen detection.

Therefore, one aspect of the present disclosure provides HCV core antigen polypeptides for use as sero-markers, for examples, as sero-markers for early detection of HCV infection. Specific embodiments of HCV core antigen polypeptides comprise a polypeptide having all or part of the sequence of Accession Nos. of ABM14502, BAM14497, and/or AAA21062. See Bukh et al., Sequence analysis of the core gene of 14 hepatitis C virus genotypes, Proc Natl Acad Sci USA, 1994, 91(17):8239-43.

Other specific embodiments of HCV core antigen polypeptides comprise a polypeptide having all or part of the amino acid sequence of SEQ ID NO: 1.

(SEQ ID NO: 1)
MSTNPKPQRKTKRNTNRRPQDVKFPGGGQIVGGVYLLPRRGPRLGVRATR
KTSERSQPRGRRQPIPKARQPEGRAWAQPGYPWPLYGNEGMGWAGWLLSP
RGSRPSWGPTDPRRRSRNLGKVIDTLTCGFADLMGYIPLVGAPLGGAARA
LAHGVRVLEDGVNYATGNLPGCSFSIFLLALLSCLTIPASA.

Other embodiments of HCV core antigen polypeptides comprise a polypeptide having all or part of the amino acid sequence of:

MSTNPKPQRC (SEQ ID NO: 2), and/or

TKRNTNRRPC (SEQ ID NO: 3), and/or

DVKFPGGGQIVGGVYCRR (SEQ ID NO: 4), and/or

CLLPRRGPRLGVRA (SEQ ID NO: 5), and/or

CRKTSERSQPRGRRQPIPK (SEQ ID NO: 6), and/or

RKRCWAQPGYPWPLY (SEQ ID NO: 7), and/or

RKRCGWAGWLLSP (SEQ ID NO: 8), and/or

DPRRRSRNLGKVIDTLTC (SEQ ID NO: 9), and/or

RKRCGFADLMGYIPLVGAP (SEQ ID NO: 10), and/or

EDGVNYATGNLPGCK (SEQ ID NO: 11), in any suitable combination and order.

In one aspect, the underlined amino acid residues in SEQ ID NOs: 2-8 and 10-11 are added for increased solubility. In some aspects, the HCV core antigen polypeptide of the present disclosure comprises a polypeptide having all or part of an amino acid sequence without the underlined amino acid residue(s) in SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 10, or SEQ ID NO: 11.

Alternatively, embodiments of HCV core antigen polypeptides comprise variant, homolog or analog polypeptides that have alterations in the amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, and/or SEQ ID NO: 11.

Table 1 lists SEQ ID NOs: 2-11. All listed peptide sequences are common to both the 1a and 1b genotypes.

| | | | Referred in | Common | SEQ ID |
|---|---|---|---|---|---|
| Name | Sequence | Position | Bukh et al. | genotype | NO |
| CV001 | 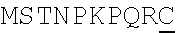 | 1-9 | 1-8 | 1a, 1b, 2a, 2b | 2 |
| CV011 | 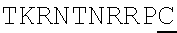 | 11-19 | | 1a, 1b, 2a, 2b | 3 |
| CV021 | 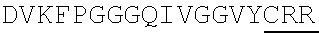 | 21-35 | 22-27 | 1a, 1b, 2a, 2b | 4 |
| CV036 | 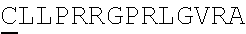 | 36-48 | | 1a, 1b, 2a, 2b | 5 |
| CV050 | 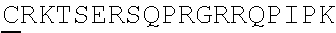 | 50-67 | | 1a, 1b, 2a, 2b | 6 |
| CV076 | 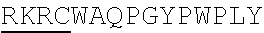 | 76-86 | | 1a, 1b, | 7 |
| CV092 | 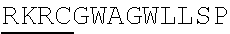 | 92-100 | | 1a, 1b, 2a, 2b | 8 |
| CV121 | DPRRRSRNLGKVIDTLTC | 111-128 | 110-125 | 1a, 1b, one | 9 |
| | | | | difference in | |
| | | | | 2a or 2b | |
| CV128 | 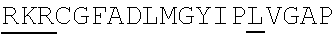 | 128-143 | 131-141 | 1a, 1b, 1a, 1b, | 10 |
| | | | | one difference | |
| | | | | in 2a or 2b | |
| CV159 | 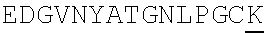 | 159-172 | | 1a, 1b, 1a, 1b, | 11 |
| | | | | one difference | |
| | | | | in 2a or 2b | |

In some aspects, variants, homologs, or analogs of HCV core antigen polypeptides share a high degree of structural identity and homology (e.g., 90% or more homology). In some aspects, an HCV core antigen polypeptide contains conservative amino acid substitutions within the HCV core antigen peptide sequences described herein or contain a substitution of an amino acid from a corresponding position in a homologue of HCV core antigen peptide. In comparisons of protein sequences, the terms, similarity, identity, and homology each have a distinct meaning as appreciated in the field of genetics. Moreover, orthology and paralogy can be important concepts describing the relationship of members of a given protein family in one organism to the members of the same family in other organisms.

Conservative amino acid substitutions can frequently be made in a protein or peptide without altering either the conformation or the function of the protein or peptide. Peptides of the present disclosure can comprise 1, 2, 3, 4, 5, 6, 7, 8, 9, 10, 11, 12, 13, 14, 15 or more conservative substitutions. Such changes include substituting any of isoleucine (I), valine (V), and leucine (L) for any other of these hydrophobic amino acids; aspartic acid (D) for glutamic acid (E) and vice versa; glutamine (Q) for asparagine (N) and vice versa; and serine (S) for threonine (T) and vice versa. Other substitutions can also be considered conservative, depending on the environment of the particular amino acid and its role in the three-dimensional structure of the protein or peptide. For example, glycine (G) and alanine (A) can frequently be interchangeable, as can alanine (A) and valine (V). Methionine (M), which is relatively hydrophobic, can frequently be interchanged with leucine and isoleucine, and sometimes with valine. Lysine (K) and arginine (R) are frequently interchangeable in locations in which the significant feature of the amino acid residue is its charge and the differing pKs of these two amino acid residues are not significant. Still other changes can be considered “conservative” in particular environments (see, e.g. pages 13-15 “Biochemistry” 2nd ED. Lubert Stryer ed (Stanford University); Henikoff et al., PNAS, 1992, 89:10915-19; Lei et al., J Biol Chem, 1995, 270(20): 11882-86).

Embodiments of the present disclosure include a wide variety of art-accepted variants or analogs of HCV core antigen such as polypeptides having amino acid insertions, deletions and substitutions. HCV core antigen polypeptides, including variants thereof, can be made using methods known in the art such as site-directed mutagenesis, alanine scanning, and PCR mutagenesis. Site-directed mutagenesis (Carter et al., Nucl. Acids Res., 13:4331 (1986); Zoller et al., Nucl. Acids Res., 10:6487 (1987)), cassette mutagenesis (Wells et al., Gene, 34:315 (1985)), restriction selection mutagenesis (Wells et al., Philos. Trans. R. Soc. London SerA, 317:415 (1986)) or other known techniques can be performed on the cloned DNA to produce variants of the HCV core antigen DNA.

Scanning amino acid analysis can also be employed to identify one or more amino acids along a contiguous sequence that is involved in a specific biological activity such as a protein-protein interaction. Among the preferred scanning amino acids are relatively small, neutral amino acids. Such amino acids include alanine, glycine, serine, and cysteine. Alanine is typically a preferred scanning amino acid among this group because it eliminates the side-chain beyond the beta-carbon and is less likely to alter the main-chain conformation of the variant. Alanine is also typically preferred because it is the most common amino acid. Further, it is frequently found in both buried and exposed positions (Creighton, The Proteins, (W.H. Freeman & Co., N.Y.); Chothia, J. Mol. Biol., 150:1 (1976)). If alanine substitution does not yield adequate amounts of variant, an isosteric amino acid can be used.

In some aspects, HCV core antigen polypeptides disclosed herein, including variants, analogs or homologs thereof, have the distinguishing attribute of having at least one epitope that is “cross reactive” with the HCV core antigen amino acid sequence set forth in SEQ ID NO: 1. As used in this sentence, “cross reactive” means that an antibody or T cell that specifically binds to an HCV core antigen polypeptide also specifically binds to the HCV core antigen having the amino acid sequence of SEQ ID NO: 1. A polypeptide ceases to be a variant of the amino acid sequence of SEQ ID NO: 1, when it no longer contains any epitope capable of being recognized by an antibody or T cell that specifically binds to the HCV core antigen. Those skilled in the art understand that antibodies that recognize proteins bind to epitopes of varying size, and a grouping of the order of about four or five amino acids, contiguous or not, is regarded as a typical number of amino acids in a minimal epitope. See, e.g., Nair et al., J. Immunol 2000 165(12): 6949-6955; Hebbes et al., Mol Immunol (1989) 26(9):865-73; Schwartz et al., J Immunol (1985) 135(4):2598-608.

In some embodiments, an HCV core antigen antibody disclosed herein specifically binds to all or a portion of one or more sequences (amino acid sequence or nucleic acid sequence) of the following accession numbers: AB030907, AB031663, AB047639, AB092962, AB520610, AB705379, AF009606, AF011751, AF011752, AF011753, AF046866, AF064490, AF238486, AF268569, AF268570, AF268571, AF268572, AF268573, AF268574, AF268575, AF268576, AF268577, AF268578, AF268579, AF268580, AF271632, AF290978, AF359345, AF359346, AF359347, AF359348, AF359349, AF511948, AF511949, AF511950, AF512996, AF529293, AJ278830, AJ557443, AJ557444, AM910652, AY051292, AY231582, AY365214, AY521893, AY521894, AY521895, AY521896, AY521897, AY521898, AY521899, AY521900, AY521901, AY521902, AY521903, AY521904, AY521905, AY521906, AY521907, AY521908, AY521909, AY521910, AY521911, AY521912, AY521913, AY521914, AY521915, AY521916, AY521917, AY521918, AY521919, AY521920, AY521921, AY521922, AY521923, AY521924, AY521925, AY521926, AY521927, AY521928, AY521929, AY521930, AY521931, AY521932, AY521933, AY521934, AY521957, AY521958, AY521959, AY521960, AY521961, AY521962, AY521963, AY521964, AY521965, AY521966, AY521967, AY521968, AY521969, AY521970, AY521971, AY521972, AY521973, AY521974, AY521975, AY521976, AY521977, AY521978, AY521979, AY521980, AY521982, AY521983, AY521984, AY521985, AY521986, AY521987, AY521988, AY521989, AY521990, AY521991, AY521992, AY521993, AY521994, AY521995, AY521996, AY521997, AY521998, AY521999, AY522000, AY522001, AY522002, AY522003, AY522004, AY522005, AY522006, AY522007, AY522008, AY522010, AY522034, AY522035, AY522036, AY522037, AY522038, AY522039, AY522040, AY522041, AY522042, AY522043, AY522044, AY522045, AY522046, AY522047, AY522048, AY522049, AY522050, AY522051, AY522052, AY522053, AY522054, AY522055, AY522056, AY522057, AY522058, AY522059, AY522060, AY522061, AY522062, AY522063, AY522064, AY522065, AY522066, AY522067, AY522068, AY522069, AY522070, AY522071, AY522072, AY522073, AY522074, AY522075, AY522076, AY522077, AY522078, AY522091, AY522092, AY522093, AY522094, AY522095, AY522096, AY522098, AY522099, AY522100, AY522123, AY522124, AY522125, AY522126, AY522127, AY522128, AY522129, AY522130, AY522131, AY522132, AY522133, AY522134, AY522135, AY522136, AY522137, AY522138, AY522139, AY522140, AY522141, AY522142, AY522143, AY522144, AY522145, AY522146, AY522147, AY522148, AY522149, AY522150, AY522151, AY522152, AY522153, AY522154, AY522155, AY522156, AY522157, AY522158, AY522171, AY522172, AY522173, AY522174, AY522175, AY522176, AY522177, AY522178, AY522179, AY522180, AY522181, AY522182, AY522183, AY522184, AY522185, AY522186, AY522187, AY522188, AY522189, AY522190, AY522191, AY522192, AY522193, AY522194, AY522195, AY522196, AY522197, AY522198, AY522199, AY522200, AY522201, AY522202, AY522203, AY522204, AY522205, AY522206, AY522207, AY522208, AY522209, AY522210, AY522211, AY522212, AY522213, AY522214, AY522215, AY522216, AY522217, AY522218, AY522219, AY522220, AY522221, AY522222, AY522223, AY522224, AY522225, AY522226, AY522227, AY522228, AY522229, AY522230, AY522231, AY522232, AY522233, AY522234, AY522252, AY522253, AY522254, AY522255, AY522256, AY522257, AY522258, AY522259, AY522260, AY522261, AY522262, AY522263, AY522264, AY522265, AY522266, AY522267, AY522268, AY522269, AY522270, AY522271, AY522272, AY522273, AY522274, AY522276, AY522278, AY522280, AY522281, AY522282, AY522283, AY522294, AY522295, AY522296, AY522297, AY522298, AY522299, AY522300, AY522301, AY522302, AY522303, AY522304, AY522305, AY522306, AY522307, AY522308, AY522309, AY522310, AY522311, AY522312, AY522313, AY522314, AY522315, AY522316, AY522317, AY522318, AY522319, AY522320, AY522321, AY522322, AY522323, AY522324, AY522325, AY522326, AY587016, AY615798, AY695436, AY695437, AY746460, AY859526, AY878652, AY885238, AY956463, AY956464, AY956465, AY956466, AY956468, AY956469, D00831, D00944, D10749, D10988, D11355, D14853, D17763, D49374, D50409, D63821, D63822, D84262, D84263, D84264, D84265, DQ061300, DQ061301, DQ061302, DQ061303, DQ061304, DQ061305, DQ061306, DQ061307, DQ061308, DQ061309, DQ061310, DQ061311, DQ061312, DQ061313, DQ061314, DQ061315, DQ061316, DQ061317, DQ061318, DQ061319, DQ061320, DQ061321, DQ061322, DQ061323, DQ061324, DQ061325, DQ061326, DQ061327, DQ061328, DQ061329, DQ061330, DQ077736, DQ155561, DQ278891, DQ278892, DQ278893, DQ314805, DQ314806, DQ418786, DQ418788, DQ430811, DQ430813, DQ480513, DQ516083, DQ835760, DQ835761, DQ835762, DQ835763, DQ835764, DQ835765, DQ835766, DQ835769, DQ835770, DQ838739, DQ838740, DQ838741, DQ838742, DQ838743, DQ838744, DQ838745, EF032883, EF032884, EF032885, EF032886, EF032887, EF032889, EF032890, EF032891, EF032892, EF032895, EF032896, EF032898, EF032899, EF032900, EF108306, EF407411, EF407412, EF407413, EF407414, EF407415, EF407416, EF407417, EF407418, EF407419, EF407420, EF407421, EF407422, EF407423, EF407424, EF407425, EF407426, EF407427, EF407428, EF407429, EF407430, EF407431, EF407432, EF407433, EF407434, EF407435, EF407436, EF407437, EF407438, EF407439, EF407440, EF407441, EF407442, EF407443, EF407444, EF407445, EF407446, EF407447, EF407448, EF407449, EF407450, EF407451, EF407452, EF407453, EF407454, EF407455, EF407456, EF407457, EF424625, EF424626, EF424627, EF424628, EF424629, EF560518, EF560519, EF560520, EF560521, EF560522, EF560523, EF560524, EF560525, EF560526, EF560527, EF560528, EF560529, EF560530, EF560535, EF560536, EF560537, EF560538, EF560539, EF560540, EF560541, EF560542, EF560543, EF560544, EF560545, EF560546, EF560547, EF560548, EF560549, EF560550, EF560555, EF560556, EF560557, EF560558, EF589160, EF589161, EF621489, EF632069, EF632070, EF632071, EU155213, EU155214, EU155215, EU155216, EU155233, EU155236, EU155237, EU155238, EU155239, EU155240, EU155241, EU155242, EU155243, EU155244, EU155245, EU155246, EU155247, EU155248, EU155249, EU155250, EU155251, EU155252, EU155265, EU155266, EU155267, EU155268, EU155269, EU155270, EU155271, EU155272, EU155273, EU155274, EU155275, EU155276, EU155277, EU155278, EU155282, EU155283, EU155284, EU155285, EU155286, EU155287, EU155288, EU155289, EU155290, EU155291, EU155292, EU155293, EU155294, EU155295, EU155296, EU155297, EU155298, EU155299, EU155309, EU155310, EU155311, EU155312, EU155313, EU155314, EU155319, EU155320, EU155321, EU155322, EU155323, EU155338, EU155339, EU155340, EU155341, EU155342, EU155343, EU155344, EU155345, EU155346, EU155347, EU155348, EU155349, EU155350, EU155351, EU155352, EU155353, EU155354, EU155355, EU155378, EU155379, EU155380, EU158186, EU234063, EU234064, EU234065, EU239713, EU239715, EU239716, EU246931, EU246932, EU246933, EU246934, EU246935, EU246936, EU246937, EU246938, EU246939, EU246940, EU250017, EU255927, EU255928, EU255929, EU255930, EU255931, EU255932, EU255933, EU255934, EU255935, EU255936, EU255937, EU255938, EU255939, EU255940, EU255941, EU255942, EU255943, EU255944, EU255945, EU255946, EU255947, EU255948, EU255949, EU255950, EU255951, EU255952, EU255953, EU255954, EU255955, EU255956, EU255957, EU255958, EU255959, EU255963, EU255964, EU255965, EU255966, EU255967, EU255968, EU255969, EU255970, EU255971, EU255973, EU255974, EU255975, EU255976, EU255977, EU255978, EU255979, EU255980, EU255981, EU255982, EU255983, EU255984, EU255985, EU255986, EU255987, EU255988, EU255989, EU255990, EU255991, EU255992, EU255993, EU255994, EU255995, EU255996, EU255997, EU255998, EU255999, EU256002, EU256003, EU256004, EU256005, EU256006, EU256007, EU256008, EU256009, EU256010, EU256011, EU256012, EU256013, EU256014, EU256015, EU256016, EU256017, EU256018, EU256019, EU256020, EU256021, EU256022, EU256023, EU256024, EU256025, EU256026, EU256027, EU256028, EU256029, EU256030, EU256031, EU256032, EU256033, EU256034, EU256035, EU256036, EU256037, EU256038, EU256039, EU256040, EU256041, EU256042, EU256043, EU256044, EU256046, EU256047, EU256048, EU256049, EU256050, EU256051, EU256052, EU256053, EU256055, EU256056, EU256057, EU256058, EU256060, EU256067, EU256068, EU256069, EU256070, EU256071, EU256072, EU256073, EU256074, EU256086, EU256087, EU256094, EU256095, EU256096, EU256097, EU256104, EU256105, EU256106, EU256107, EU260395, EU260396, EU362876, EU362877, EU362878, EU362879, EU362880, EU362881, EU362882, EU362883, EU362884, EU362885, EU362886, EU362887, EU362888, EU362889, EU362890, EU362891, EU362892, EU362893, EU362894, EU362895, EU362896, EU362897, EU362898, EU362899, EU362900, EU362901, EU362902, EU362903, EU362904, EU362905, EU362906, EU362907, EU362908, EU362909, EU362910, EU362911, EU370586, EU370587, EU370588, EU370589, EU370590, EU370591, EU370592, EU370593, EU370594, EU370595, EU370616, EU370617, EU370618, EU370619, EU370620, EU370621, EU370622, EU370623, EU370624, EU370625, EU370636, EU370637, EU370638, EU370639, EU370640, EU370641, EU370642, EU370643, EU370644, EU370645, EU370656, EU370657, EU370658, EU370659, EU370660, EU370661, EU370662, EU370663, EU370664, EU370665, EU392169, EU392170, EU392171, EU392172, EU392173, EU392174, EU392175, EU408326, EU408327, EU408328, EU408329, EU408330, EU408331, EU408332, EU482831, EU482832, EU482834, EU482835, EU482836, EU482837, EU482838, EU482840, EU482841, EU482842, EU482843, EU482844, EU482845, EU482846, EU482847, EU482848, EU482850, EU482852, EU482853, EU482854, EU482855, EU482856, EU482857, EU482858, EU482861, EU482862, EU482863, EU482864, EU482865, EU482866, EU482867, EU482868, EU482869, EU482870, EU482871, EU482872, EU482873, EU482876, EU482878, EU482882, EU482884, EU482887, EU482889, EU529676, EU529677, EU529678, EU529679, EU529680, EU529681, EU569722, EU569723, EU595697, EU595698, EU595699, EU643834, EU643836, EU660383, EU660384, EU660385, EU660387, EU677247, EU677248, EU677249, EU677250, EU677251, EU677252, EU677253, EU677254, EU677255, EU677256, EU677257, EU677258, EU687193, EU687194, EU687195, EU781746, EU781747, EU781748, EU781749, EU781750, EU781751, EU781752, EU781753, EU781754, EU781755, EU781756, EU781757, EU781758, EU781759, EU781760, EU781761, EU781762, EU781763, EU781764, EU781765, EU781766, EU781767, EU781768, EU781769, EU781770, EU781771, EU781772, EU781773, EU781774, EU781775, EU781776, EU781777, EU781778, EU781779, EU781780, EU781781, EU781782, EU781783, EU781784, EU781785, EU781786, EU781787, EU781788, EU781789, EU781790, EU781791, EU781792, EU781793, EU781794, EU781795, EU781796, EU781797, EU781798, EU781799, EU781800, EU781801, EU781802, EU781803, EU781804, EU781805, EU781806, EU781807, EU781808, EU781809, EU781810, EU781811, EU781812, EU781813, EU781814, EU781815, EU781816, EU781817, EU781818, EU781819, EU781820, EU781821, EU781822, EU781823, EU781824, EU798760, EU798761, EU862823, EU862824, EU862825, EU862826, EU862827, EU862828, EU862829, EU862831, EU862832, EU862833, EU862834, EU862836, EU862838, EU862839, EU862840, EU862841, FJ024087, FJ024274, FJ024275, FJ024276, FJ024278, FJ024280, FJ024281, FJ024282, FJ025854, FJ025855, FJ025856, FJ181999, FJ182000, FJ182001, FJ205867, FJ205868, FJ205869, FJ390394, FJ390395, FJ390399, FJ407092, FJ410172, FJ435090, FJ462431, FJ462432, FJ462433, FJ462434, FJ462435, FJ462436, FJ462437, FJ462438, FJ462439, FJ462440, FJ462441, FJ477239, FJ839869, FJ839870, FJ911707, FJ911708, FJ911709, FJ911712, FJ911713, FJ911714, FJ911718, FJ911719, FJ911720, FJ911721, FJ911722, FJ911723, FJ911724, FJ911725, FJ911727, FJ911728, FJ911729, FJ911730, FJ911731, FJ911732, FJ911734, FJ911735, FJ911736, FN666290, FN666291, FN666292, FN666293, FN666294, FN666295, FN666296, FN666297, FN666298, FN666299, FN666300, FN666301, FN666302, FN666303, FN666304, FN666305, FN666306, FN666307, FN666308, FN666309, FN666310, FN666311, FN666312, FN666313, FN666314, FN666315, FN666316, FN666317, FN666318, FN666319, FN666320, FN666321, FN666322, FN666323, FN666324, FN666326, FN666374, FN666375, FN666376, FN666377, FN666378, FN666379, FN666380, FN666381, FN666382, FN666383, FN666384, FN666385, FN666386, FN666387, FN666388, FN666389, FN666390, FN666391, FN666392, FN666393, FN666394, FN666395, FN666396, FN666397, FN666398, FN666399, FN666400, FN666401, FN666402, FN666403, FN666404, FN666405, FN666406, FN666407, FN666408, FN666428, FN666429, GQ149768, GQ451336, GQ848648, GQ848649, GQ848650, GQ848651, GQ848652, GQ848653, GQ848654, GQ848655, GQ848656, GQ848698, GQ848699, GQ848700, GQ848701, GQ848702, GQ848703, GQ848704, GQ848705, GQ848747, GQ848748, GQ848751, GQ848752, GQ848781, GQ848782, GQ848783, GQ848784, GQ848785, GQ848786, GQ848787, GQ848788, GQ848789, GQ848790, GQ848791, GQ848792, GQ848793, GQ848794, GQ848795, GQ848796, GQ848797, GQ848798, GQ870457, GQ870459, GQ870460, GQ870461, GQ870462, GQ870463, GQ870464, GQ870465, GQ870466, GQ870467, GQ870468, GQ870469, GQ870470, GQ870471, GQ870472, GQ870473, GQ870474, GQ870475, GQ870476, GQ870477, GQ870478, GQ870479, GQ870480, GQ870481, GQ870482, GQ870483, GQ870484, GQ870485, GQ870487, GQ870488, GQ870489, GQ870490, GQ870491, GQ870492, GQ870493, GQ870494, GQ870495, GQ870496, GQ870497, GQ870498, GQ870500, GQ870501, GQ870502, GQ870504, GQ870505, GQ870506, GQ870507, GQ870508, GQ870509, GQ913854, GQ913855, GQ913856, GQ913857, HM000514, HM000515, HM000516, HM000517, HM000518, HM000519, HM000520, HM000521, HM000522, HM000523, HM000524, HM000525, HM000526, HM000527, HM000528, HM000529, HM000530, HM000531, HM000532, HM000533, HM000534, HM000535, HM000536, HM000537, HM000538, HM000539, HM000540, HM000541, HM000542, HM041973, HM041974, HM041975, HM041976, HM041977, HM041978, HM041979, HM041980, HM041981, HM041982, HM041983, HM041984, HM777358, HM777359, HQ113464, HQ113465, HQ113466, HQ113467, HQ113468, HQ13469, HQ113494, HQ113495, HQ113496, HQ113497, HQ113498, HQ113499, HQ113524, HQ113525, HQ113526, HQ113527, HQ113528, HQ113529, HQ113560, HQ113561, HQ113562, HQ113563, HQ113564, HQ113565, HQ113606, HQ113607, HQ113608, HQ113609, HQ113610, HQ113611, HQ113636, HQ113637, HQ113638, HQ113639, HQ113640, HQ113641, HQ113666, HQ113667, HQ113668, HQ113669, HQ113670, HQ113671, HQ113726, HQ113727, HQ113728, HQ113729, HQ113730, HQ113731, HQ537007, HQ537008, HQ537009, HQ828059, JQ061778, JQ061780, JQ061781, JQ061782, JQ061783, JQ061784, JQ061785, JQ061786, JQ061787, JQ061887, JQ061888, JQ061889, JQ061890, JQ061891, JQ061892, JQ062043, JQ062044, JQ062045, JQ062046, JQ062047, JQ062048, JQ062049, JQ062050, JQ062051, JQ062052, JQ343803, JQ343804, JQ343805, JQ343806, JQ343807, JQ343808, JQ343809, 343809, JQ343810, JQ343810, JQ343811, JQ343812, JQ343813, JQ343814, JQ343815, JQ343816, JQ343817, JQ343818, JQ343819, JQ343820, JQ343821, JQ343822, JQ343823, JQ343824, JQ343825, JQ343826, JQ914271, JQ914272, JQ914273, JQ914272, JQ914273, JQ914274, JQ924876, JQ924877, JQ924878, JQ924879, JQ924880, JQ924881,82, 24882, JQ924883, JQ924885, JQ924887, JX463525, JX463526, JX463527, JX463528, JX463529, JX463530, JX463531, JX463532, JX463533, JX463534, JX463535, JX463536, JX463537, JX463538, JX463539, JX463540, JX463541, JX463542, JX463543, JX463544, JX463545, JX463546, JX463547, JX463548, JX463549, JX463550, JX463551, JX463552, JX463553, JX463554, JX463555, JX463556, JX463557, JX463558, JX463559, JX463560, JX463561, JX463562, JX463563, JX463564, JX463565, JX463566, JX463567, JX463568, JX463569, JX463570, JX463571, JX463572, JX463573, JX463574, JX463575, JX463576, JX463577, JX463578, JX463579, JX463580, JX463581, JX463582, JX463583, JX463584, JX463585, JX463586, JX463587, JX463588, JX463589, JX463590, JX463591, JX463592, JX463593, JX463594, JX463595, JX463596, JX463597, JX463598, JX463599, JX463600, JX463601, JX463602, JX463603, JX463604, JX463605, JX463606, JX463607, JX463608, JX463609, JX463610, JX463611, JX463612, JX463613, JX463614, JX463615, JX463616, JX463617, JX463618, JX463619, JX463620, JX463621, JX463622, JX463623, JX463624, JX463625, JX463626, JX463627, JX463628, JX463629, JX463630, JX463631, JX463632, JX463633, JX463634, JX463635, JX463636, JX463637, JX463638, JX463639, JX463640, JX463641, KC143868, KC143869, KC143870, KC143871, KC143872, KC143873, KC143874, KC143875, KC143876, KC143877, KC143878, KC143879, KC143916, KC143917, KC143918, KC143919, KC143920, KC143921, KC155254, KC283194, KC844049, L12353, M55970, M62321, M62381, M62382, M67463, M74804, M74805, M74808, M74811, M74812, NC_004102, NC_009826, U10194, U10196, U10206, U10207, U10222, U10232, U55281, U55283, U55285, X76918, X84079, Y11604, Y12083, AB008441, AB008442, AB008443, AB008445, AB008446, AB008447, AB014487, AB016785, AB030159, AB030160, AB030161, AB030162, AB030163, AB030164, AB030165, AB030166, AB030167, AB030168, AB030169, AB030170, AB030171, AB030172, AB030173, AB030907, AB031663, AB037249, AB039866, AB039869, AB039870, AB039871, AB039872, AB039873, AB039874, AB039875, AB039876, AB039877, AB047639, AB049087, AB049088, AB049089, AB049090, AB049091, AB049092, AB049093, AB049094, AB049095, AB049096, AB049097, AB049098, AB049099, AB049100, AB049101, AB061927, AB061928, AB061929, AB061930, AB061931, AB061932, AB061933, AB061934, AB061935, AB061936, AB061937, AB061938, AB061939, AB061940, AB061941, AB061942, AB061943, AB061944, AB061945, AB061946, AB061947, AB061948, AB061949, AB061950, AB061951, AB061952, AB061953, AB061954, AB061955, AB061956, AB061957, AB061958, AB061959, AB061960, AB061961, AB061962, AB061963, AB061964, AB061965, AB061966, AB061967, AB061968, AB061969, AB061970, AB061971, AB061972, AB061973, AB061974, AB061975, AB061976, AB061977, AB061978, AB061979, AB061980, AB061981, AB061982, AB061983, AB061984, AB061985, AB061986, AB061987, AB061988, AB061989, AB061990, AB061991, AB061992, AB061993, AB061994, AB061995, AB061996, AB061997, AB061998, AB061999, AB062000, AB062001, AB062002, AB062003, AB062004, AB062005, AB062006, AB062007, AB062008, AB062009, AB062010, AB062011, AB062012, AB062013, AB062014, AB062015, AB062016, AB062017, AB062018, AB062019, AB062020, AB062021, AB062022, AB062023, AB062024, AB062025, AB062026, AB062173, AB062174, AB062175, AB062176, AB062177, AB062178, AB062179, AB062180, AB062181, AB062182, AB062183, AB062184, AB062185, AB062186, AB062187, AB062188, AB062189, AB062190, AB062191, AB062192, AB062193, AB062194, AB062195, AB062196, AB062197, AB062198, AB062199, AB062200, AB062201, AB062202, AB062203, AB062204, AB062205, AB062206, AB062207, AB062208, AB062209, AB062210, AB062211, AB062212, AB062213, AB062214, AB062215, AB062216, AB062217, AB062218, AB062219, AB062220, AB062221, AB062222, AB062223, AB062224, AB062225, AB062226, AB062227, AB062228, AB062229, AB062230, AB062231, AB062232, AB062233, AB062234, AB062235, AB062236, AB062237, AB062238, AB062239, AB062240, AB062241, AB062242, AB062243, AB062244, AB062245, AB062246, AB062247, AB062248, AB062249, AB062250, AB062251, AB062252, AB062253, AB062254, AB062255, AB062256, AB062257, AB062258, AB062259, AB062260, AB062261, AB062262, AB062263, AB062264, AB062265, AB062266, AB062267, AB062268, AB062269, AB062270, AB062271, AB062272, AB063485, AB063486, AB077701, AB077702, AB077703, AB077704, AB077705, AB077706, AB077707, AB077708, AB077709, AB077710, AB077711, AB077712, AB077713, AB077714, AB077715, AB077716, AB077717, AB077718, AB077719, AB077720, AB077721, AB077722, AB077723, AB077724, AB077725, AB077726, AB077727, AB077728, AB077729, AB077730, AB077731, AB077732, AB077733, AB077734, AB077735, AB077736, AB077737, AB077738, AB077739, AB077740, AB077741, AB077742, AB077743, AB077744, AB077745, AB077746, AB077747, AB080299, AB086043, AB086044, AB086045, AB086046, AB086047, AB086048, AB086049, AB086050, AB086051, AB086052, AB086053, AB086054, AB119282, AB154177, AB154178, AB154179, AB154180, AB154181, AB154182, AB154183, AB154184, AB154185, AB154186, AB154187, AB154188, AB154189, AB154190, AB154191, AB154192, AB154193, AB154194, AB154195, AB154196, AB154197, AB154198, AB154199, AB154200, AB154201, AB154202, AB154203, AB154204, AB154205, AB154206, AB162859, AB162860, AB162861, AB191333, AB249644, AB269350, AB269351, AB269352, AB291002, AB291012, AB291014, AB291015, AB291019, AB291024, AB291028, AB301744, AB301745, AB301746, AB301747, AB301748, AB301749, AB301750, AB301751, AB301752, AB301753, AB301754, AB301755, AB301756, AB301757, AB301758, AB301759, AB301760, AB301761, AB301762, AB301763, AB301764, AB306333, AB306334, AB306338, AB306345, AB306350, AB306357, AB306360, AB306367, AB306372, AB306373, AB306374, AB306382, AB306395, AB306404, AB426117, AB429050, AB435162, AB442219, AB442220, AB442221, AB442222, AB492181, AB492182, AB492183, AB492184, AB492185, AB492186, AB492187, AB492188, AB492189, AB492190, AB492191, AB492192, AB492193, AB492194, AB492195, AB492196, AB492197, AB492198, AB492199, AB492200, AB492201, AB492202, AB492203, AB492204, AB492205, AB492206, AB492207, AB492208, AB492209, AB492210, AB492211, AB492212, AB492213, AB492214, AB492215, AB492216, AB492217, AB492218, AB492219, AB492220, AB492221, AB492222, AB492223, AB492224, AB492225, AB492226, AB492227, AB492228, AB492229, AB492230, AB492231, AB492232, AB492233, AB492234, AB492235, AB518796, AB518797, AB518798, AB518799, AB518800, AB518801, AB518802, AB518803, AB518804, AB518805, AB518806, AB518807, AB518808, AB518809, AB518810, AB518811, AB518812, AB518813, AB518814, AB518815, AB518816, AB518817, AB518818, AB518819, AB518820, AB518821, AB518822, AB518823, AB518824, AB518825, AB518826, AB518827, AB518828, AB518829, AB518830, AB518831, AB518832, AB518833, AB518834, AB518835, AB518836, AB518837, AB518838, AB518839, AB518840, AB518841, AB518842, AB518843, AB518844, AB518845, AB518846, AB518847, AB518848, AB518849, AB518850, AB518851, AB518852, AB518853, AB518854, AB518855, AB518856, AB518857, AB518858, AB518859, AB518860, AB518861, AB522972, AB522984, AB522985, AB522993, AB522996, AB523000, AB523003, AB523005, AB523010, AB523012, AB523014, AB523023, AB523025, AB523035, AB523037, AB523040, AB523041, AB523045, AB523048, AB523053, AB523060, AB523063, AB523064, AB523065, AB523074, AB523079, AB523084, AB523087, AB523093, AB523096, AB523097, AB523104, AB523108, AB523115, AB523116, AB523120, AB523141, AB523149, AB523155, AB523157, AB691953, AB700211, AB700212, AB700213, AB700214, AB700215, AB700216, AB700217, AB700218, AB700219, AB700220, AB700221, AB700222, AB700223, AB700224, AB700225, AB700226, AB700227, AB700228, AB700229, AB700230, AB700231, AB700232, AB700233, AB700234, AB700235, AB700236, AB700237, AB700238, AB700239, AB700240, AB700241, AB700242, AB700243, AB700244, AB700245, AB705317, AB705324, AB705325, AB705326, AB705327, AB705328, AB705330, AB705331, AB705333, AB705340, AB705341, AB705342, AB705343, AB705344, AB705348, AB705349, AB705350, AB705352, AB705353, AB705354, AB705357, AB705361, AB705362, AB705363, AB705364, AB705366, AB705374, AB705380, AB705381, AB705383, AB705386, AB705388, AB705389, AB705390, AB705391, AB719460, AB719461, AB719462, AB719463, AB719464, AB719465, AB719466, AB719467, AB719468, AB719469, AB719470, AB719471, AB719472, AB719473, AB719474, AB719475, AB719476, AB719477, AB719478, AB719479, AB719480, AB719481, AB719482, AB719483, AB719484, AB719485, AB719486, AB719487, AB719488, AB719489, AB719490, AB719491, AB719492, AB719493, AB719494, AB719495, AB719496, AB719497, AB719498, AB719499, AB719500, AB719501, AB719502, AB719503, AB719504, AB719505, AB719506, AB719507, AB719508, AB719509, AB719510, AB719511, AB719512, AB719513, AB719514, AB719515, AB719516, AB719517, AB719518, AB719519, AB719520, AB719521, AB719522, AB719523, AB719524, AB719525, AB719526, AB719527, AB719528, AB719529, AB719530, AB719531, AB719532, AB719533, AB719534, AB719535, AB719536, AB719537, AB719538, AB719539, AB719540, AB719541, AB719542, AB719543, AB719544, AB719545, AB719546, AB719547, AB719548, AB719549, AB719550, AB719551, AB719552, AB719553, AB719554, AB719555, AB719556, AB719557, AB719558, AB719559, AB719560, AB719561, AB719562, AB719563, AB719564, AB719565, AB719566, AB719567, AB719568, AB719569, AB719570, AB719571, AB719572, AB719573, AB719574, AB719575, AB719576, AB719577, AB719578, AB719579, AB719580, AB719581, AB719582, AB719583, AB719584, AB719585, AB719586, AB719587, AB719588, AB719589, AB719590, AB719591, AB719592, AB719593, AB719594, AB719595, AB719596, AB779562, AB779679, AB822372, AB822373, AB822374, AB822375, AB822376, AB822377, AB822378, AB822379, AB822380, AB822381, AB822382, AB822383, AB822384, AB822385, AB822386, AB822387, AB822388, AB822389, AB822390, AB822391, AB822392, AB822393, AB822394, AB822395, AB822396, AB822397, AB822398, AB822399, AB822400, AB822401, AB822402, AB822403, AB822404, AB822405, AB822406, AB822407, AB822408, AB822409, AB822410, AB822411, AB822412, AB822413, AB822414, AB822415, AB822416, AB822417, AB822418, AB822419, AB822420, AB822421, AB822422, AB822423, AB822424, AB822425, AB822426, AB822427, AB822428, AB822429, AB822430, AB822431, AB822432, AB822433, AB822434, AB822435, AB822436, AB822437, AB822438, AB822439, AB822440, AB822441, AB822442, AB822443, AB822444, AB822445, AB822446, AB822447, AB822448, AB822449, AB822450, AB822451, AB822452, AB822453, AB822454, AB822455, AB822456, AB822457, AB822458, AB822459, AF046866, AF054247, AF054248, AF054249, AF054250, AF054251, AF054252, AF054253, AF054254, AF054255, AF054256, AF054257, AF054258, AF054259, AF064490, AF072824, AF134740, AF134742, AF134743, AF134745, AF134748, AF134749, AF139594, AF165045, AF165046, AF165047, AF165048, AF165049, AF165050, AF165051, AF165052, AF165053, AF165054, AF165055, AF165056, AF165057, AF165058, AF165059, AF165060, AF165061, AF165062, AF165063, AF165064, AF176573, AF205595, AF207752, AF207753, AF207754, AF207755, AF207756, AF207757, AF207758, AF207759, AF207760, AF207761, AF207762, AF207763, AF207764, AF207765, AF207766, AF207767, AF207768, AF207769, AF207770, AF207771, AF207772, AF207773, AF207774, AF208024, AF210895, AF210898, AF210899, AF210900, AF210901, AF213648, AF213649, AF213650, AF213651, AF213652, AF213653, AF213654, AF213894, AF213895, AF233703, AF233704, AF233713, AF233714, AF238486, AF313916, AF333324, AF350914, AF350915, AF350916, AF350917, AF356827, AF379040, AF379041, AF379042, AF379043, AF379044, AF379045, AF379046, AF379047, AF379048, AF379049, AF379050, AF379051, AF379052, AF379053, AF379054, AF379055, AF379056, AF379057, AF379058, AF379059, AF379060, AF379061, AF379062, AF379063, AF379064, AF379065, AF379066, AF379067, AF379068, AF379069, AF379070, AF379071, AF379072, AF379073, AF379074, AF379075, AF379076, AF379077, AF379078, AF379079, AF379080, AF379081, AF379082, AF379083, AF379084, AF379085, AF379086, AF379087, AF379088, AF379089, AF379090, AF379091, AF379092, AF379093, AF379094, AF379095, AF379096, AF379097, AF379098, AF379099, AF379100, AF379101, AF379102, AF379103, AF379104, AF379105, AF379106, AF379107, AF379108, AF379109, AF379110, AF379111, AF379112, AF379113, AF379114, AF379115, AF379116, AF379117, AF379118, AF482729, AF483269, AF484974, AF484975, AF506614, AF506615, AF506616, AF506617, AF506618, AF506619, AF506620, AF511950, AF525887, AF525888, AF525889, AF525890, AF525891, AF525892, AF525893, AF525894, AF531454, AF531455, AJ000009, AJ132996, AJ132997, AJ238799, AJ238800, AJ582128, AJ582129, AJ582130, AJ781122, AJ781123, AJ849973, AJ849974, AM114084, AM114085, AM114086, AM114087, AM114088, AM114089, AM114090, AM114091, AM114092, AM114093, AM114094, AM114095, AM114096, AM114097, AM114098, AM262455, AM262456, AM262457, AM262458, AM262459, AM262460, AM262461, AM262462, AM262463, AM262464, AM262465, AM262466, AM262467, AM262468, AM262469, AM262470, AM262471, AM262472, AM262473, AM262474, AM262475, AM262476, AM262477, AM262478, AM262479, AM262480, AM262481, AM262482, AM262483, AM262484, AM262485, AM262486, AM262487, AM262488, AM262489, AM262490, AM262491, AM262492, AM262493, AM262494, AM262495, AM262496, AM262497, AM262498, AM262499, AM262500, AM262501, AM262502, AM262503, AM262504, AM262505, AM262506, AM262507, AM262508, AM262509, AM262510, AM262511, AM285684, AM285685, AM285686, AM285687, AM285688, AM285689, AM285690, AM285691, AM285692, AM285693, AM285694, AM285695, AM285696, AM285697, AM285698, AM285699, AM285700, AM285701, AM285702, AM285703, AM285704, AM285705, AM285706, AM285707, AM285708, AM285709, AM285710, AM285711, AM285712, AM285713, AM285714, AM285715, AM285716, AM285717, AM285718, AM285719, AM285720, AM285721, AM285722, AM285724, AM285725, AM285726, AM285727, AM285728, AM285729, AM285730, AM285731, AM285732, AM285734, AM285735, AM285736, AM285737, AM285738, AM285739, AM285740, AM285741, AM285742, AM285743, AM285744, AM285745, AM285746, AM285747, AM285748, AM285749, AM285750, AM285751, AM285752, AM285753, AM285754, AM285755, AM285756, AM285757, AM285758, AM285759, AM285760, AM285761, AM285762, AM285763, AM285764, AM285765, AM285766, AM285767, AM285768, AM285769, AM285770, AM285771, AM285772, AM285773, AM285774, AM285775, AM285776, AM285777, AM285778, AM285779, AM285780, AM285781, AM285782, AM285783, AM285784, AM285785, AM285786, AM285787, AM285788, AM285789, AM285790, AM285791, AM285792, AM285793, AM285794, AM285795, AM285796, AM285797, AM285798, AM285799, AM285800, AM285801, AM285802, AM285803, AM285804, AM285805, AM285806, AM285807, AM285808, AM285809, AM285810, AM285811, AM285812, AM285813, AM285814, AM285815, AM285816, AM285817, AM285818, AM285819, AM285820, AM285821, AM285822, AM285823, AM285824, AM285825, AM285826, AM285827, AM285828, AM285829, AM285830, AM285831, AM285832, AM285833, AM285834, AM285835, AM285836, AM285837, AM285838, AM285839, AM285840, AM285841, AM285842, AM285843, AM285844, AM285845, AM285846, AM285847, AM285848, AM285849, AM285850, AM285851, AM285852, AM285853, AM285854, AM285855, AM285856, AM285857, AM285858, AM285859, AM285860, AM285861, AM285862, AM285863, AM285864, AM285865, AM285866, AM285867, AM285868, AM285869, AM285870, AM285871, AM285872, AM285873, AM285874, AM285875, AM285876, AM285877, AM285878, AM285879, AM285880, AM285881, AM285882, AM285883, AM285884, AM285885, AM285886, AM285887, AM285889, AM285890, AM285891, AM285892, AM285893, AM285894, AM285895, AM285896, AM285897, AM285898, AM285899, AM285900, AM285901, AM285902, AM285903, AM285904, AM285905, AM285906, AM285907, AM285908, AM285909, AM285910, AM285911, AM285912, AM285913, AM285914, AM285915, AM285917, AM285918, AM285919, AM285920, AM285921, AM285922, AM285923, AM285924, AM285925, AM285926, AM285927, AM285928, AM285929, AM285930, AM285931, AM285932, AM285933, AM285934, AM285935, AM285936, AM285937, AM285938, AM285939, AM285940, AM285941, AM285942, AM285943, AM285946, AM285947, AM285948, AM285949, AM285950, AM285951, AM285952, AM285953, AM285954, AM285955, AM285956, AM285958, AM285959, AM285960, AM285961, AM285962, AM285963, AM285964, AM285965, AM285966, AM285967, AM285968, AM285969, AM285970, AM285971, AM285972, AM285973, AM285974, AM285975, AM285976, AM285977, AM285978, AM285979, AM285980, AM285981, AM285982, AM285983, AM285984, AM285985, AM285986, AM285987, AM285988, AM285989, AM285990, AM285991, AM285992, AM285993, AM285994, AM285995, AM285996, AM285997, AM285998, AM285999, AM286000, AM286001, AM286002, AM286003, AM286004, AM286005, AM286006, AM286007, AM286008, AM286010, AM286011, AM286012, AM286013, AM286014, AM286015, AM286016, AM286017, AM286018, AM286019, AM286020, AM286021, AM286022, AM286023, AM286024, AM286025, AM286026, AM286027, AM286028, AM286029, AM286030, AM286031, AM286032, AM286034, AM286035, AM286036, AM286037, AM286038, AM286039, AM286041, AM286042, AM286043, AM286044, AM286045, AM286046, AM286047, AM286048, AM286049, AM286050, AM286051, AM286052, AM286053, AM286054, AM286055, AM286056, AM286057, AM286058, AM286059, AM286060, AM286062, AM286063, AM286065, AM286066, AM286068, AM286069, AM286070, AM286071, AM286072, AM286073, AM286074, AM286075, AM286076, AM286077, AM286078, AM286079, AM286080, AM286081, AM286082, AM286083, AM286084, AM286085, AM286086, AM286087, AM286088, AM286089, AM286090, AM286091, AM286092, AM286093, AM286094, AM286095, AM286096, AM286097, AM286098, AM286100, AM286101, AM286102, AM286103, AM286104, AM286105, AM286106, AM286107, AM286108, AM286109, AM286110, AM286111, AM286112, AM286113, AM286114, AM286115, AM286116, AM286117, AM286118, AM286119, AM286120, AM286121, AM286122, AM286123, AM286124, AM286125, AM286126, AM286127, AM286128, AM286129, AM286130, AM286131, AM286132, AM286133, AM286134, AM286135, AM286136, AM286137, AM286138, AM286139, AM286140, AM286141, AM286142, AM286143, AM286144, AM286145, AM286146, AM286147, AM286148, AM286149, AM286150, AM286151, AM286152, AM286153, AM286154, AM286155, AM286156, AM286157, AM286158, AM286159, AM286160, AM286161, AM286162, AM286163, AM286164, AM286165, AM286166, AM286167, AM286168, AM286169, AM286170, AM286171, AM286172, AM286173, AM286174, AM286175, AM286176, AM286177, AM286178, AM286179, AM286180, AM286181, AM286182, AM286183, AM286184, AM397933, AM397934, AM397935, AM397936, AM397937, AM397938, AM397939, AM397940, AM397941, AM397942, AM397943, AM397944, AM397945, AM397946, AM397947, AM397948, AM397949, AM422808, AM422809, AM422810, AM422811, AM422812, AM422813, AM422814, AM422815, AM422816, AM422817, AM422818, AM422819, AM422820, AM422821, AM422822, AM422823, AM422824, AM422825, AM422826, AM422827, AM422828, AM422829, AM494544, AM494545, AM494546, AM494547, AM494548, AM494549, AM494550, AM494551, AM494552, AM494553, AM494554, AM494555, AM494556, AM494557, AM494558, AM494559, AM494560, AM494562, AM494563, AM494564, AM494565, AM494566, AM494567, AM494568, AM494569, AM494570, AM494571, AM494572, AM494573, AM494574, AM494575, AM494577, AM494578, AM494579, AM494580, AM494581, AM494582, AM494583, AM500641, AM500642, AM500643, AM910652, AY045702, AY051292, AY070174, AY070195, AY070196, AY070197, AY070198, AY070199, AY070200, AY070201, AY070202, AY070203, AY070204, AY070205, AY070206, AY070207, AY070208, AY070209, AY070210, AY079244, AY079245, AY079246, AY079247, AY079248, AY079249, AY079250, AY079253, AY079254, AY079255, AY079256, AY079257, AY079258, AY079259, AY079260, AY079261, AY079262, AY079263, AY079265, AY079266, AY079267, AY079268, AY079269, AY079270, AY079271, AY079272, AY079273, AY079274, AY079275, AY079276, AY079277, AY079278, AY079279, AY079280, AY079281, AY079282, AY079283, AY079284, AY079286, AY089747, AY089748, AY089749, AY089750, AY089751, AY089752, AY089753, AY089764, AY190328, AY190330, AY190343, AY190344, AY190349, AY190357, AY190362, AY190364, AY190374, AY256786, AY308072, AY365213, AY446021, AY446022, AY446023, AY460204, AY506668, AY506672, AY506673, AY506680, AY506682, AY506683, AY506686, AY506690, AY506691, AY506695, AY521935, AY521936, AY521937, AY521938, AY521939, AY521940, AY521941, AY521942, AY521943, AY521944, AY521945, AY521946, AY521947, AY521948, AY521949, AY521950, AY521951, AY521952, AY521953, AY521954, AY521955, AY521956, AY521981, AY522009, AY522011, AY522012, AY522013, AY522014, AY522015, AY522016, AY522017, AY522018, AY522019, AY522020, AY522021, AY522022, AY522023, AY522024, AY522025, AY522026, AY522027, AY522028, AY522029, AY522030, AY522031, AY522032, AY522033, AY522079, AY522080, AY522081, AY522082, AY522083, AY522084, AY522085, AY522086, AY522087, AY522088, AY522089, AY522090, AY522097, AY522159, AY522160, AY522161, AY522162, AY522163, AY522164, AY522165, AY522166, AY522167, AY522168, AY522169, AY522170, AY522235, AY522236, AY522237, AY522238, AY522239, AY522240, AY522241, AY522242, AY522243, AY522244, AY522245, AY522246, AY522247, AY522248, AY522249, AY522250, AY522251, AY522275, AY522277, AY522279, AY522284, AY522285, AY522286, AY522287, AY522288, AY522289, AY522290, AY522291, AY522292, AY522293, AY548590, AY548630, AY548656, AY587016, AY587287, AY587288, AY587289, AY587290, AY587291, AY587292, AY587293, AY587294, AY587295, AY587296, AY587297, AY587298, AY587299, AY587300, AY587301, AY587302, AY587303, AY587304, AY587305, AY587306, AY587307, AY587308, AY587309, AY587310, AY587311, AY587312, AY587313, AY587314, AY587315, AY587316, AY587317, AY587318, AY587319, AY587320, AY587321, AY587322, AY587323, AY587324, AY587325, AY587326, AY587327, AY587328, AY587329, AY587330, AY587331, AY587332, AY587333, AY587334, AY587335, AY587336, AY587337, AY587338, AY587339, AY587340, AY587341, AY587342, AY587343, AY587344, AY587345, AY587346, AY587347, AY587348, AY587349, AY587350, AY587351, AY587352, AY587353, AY587354, AY587355, AY587356, AY587357, AY587358, AY587359, AY587360, AY587361, AY587362, AY587363, AY587364, AY587365, AY587366, AY587367, AY587368, AY587369, AY587370, AY587371, AY587372, AY587373, AY587374, AY587375, AY587376, AY587377, AY587378, AY587379, AY587380, AY587381, AY587382, AY587386, AY587387, AY587388, AY587389, AY587390, AY587391, AY587392, AY587393, AY587394, AY587395, AY587396, AY587397, AY587398, AY587399, AY587400, AY587401, AY587402, AY587403, AY587404, AY587405, AY587406, AY587407, AY587408, AY587409, AY587410, AY587411, AY587412, AY587413, AY587414, AY587415, AY587416, AY587417, AY587418, AY587419, AY587420, AY587421, AY587422, AY587423, AY587424, AY587425, AY587426, AY587427, AY587428, AY587429, AY587430, AY587431, AY587432, AY587433, AY587434, AY587435, AY587436, AY587437, AY587438, AY587439, AY587440, AY587441, AY587442, AY587443, AY587444, AY587445, AY587446, AY587465, AY587466, AY587467, AY587468, AY587469, AY587470, AY587471, AY587472, AY587473, AY587474, AY587475, AY587476, AY587477, AY587478, AY587479, AY587480, AY587481, AY587482, AY587483, AY587484, AY587485, AY587486, AY587487, AY587488, AY587489, AY587490, AY587491, AY587492, AY587493, AY587494, AY587495, AY587496, AY587497, AY587844, AY600628, AY600629, AY600630, AY600631, AY600632, AY600633, AY600634, AY600635, AY600636, AY600637, AY600638, AY600639, AY600640, AY600641, AY600642, AY600643, AY600644, AY600645, AY600646, AY600647, AY600648, AY600649, AY600650, AY600651, AY600652, AY600653, AY600654, AY600655, AY600656, AY600657, AY600658, AY600659, AY600660, AY600661, AY600662, AY600663, AY600664, AY600665, AY600666, AY600667, AY600668, AY600669, AY600670, AY600671, AY600672, AY600673, AY600674, AY600675, AY600676, AY600677, AY600678, AY600679, AY600680, AY600681, AY600682, AY600683, AY600684, AY600685, AY600686, AY600687, AY600688, AY600689, AY600690, AY600691, AY600692, AY600693, AY600694, AY600695, AY600696, AY600697, AY600698, AY600699, AY600700, AY600701, AY600702, AY600703, AY600704, AY600705, AY600706, AY600707, AY600708, AY600709, AY600710, AY600711, AY600712, AY600713, AY600714, AY600715, AY600716, AY600717, AY600728, AY600729, AY600730, AY600731, AY600732, AY600733, AY600734, AY600735, AY600736, AY600737, AY600738, AY600739, AY652608, AY746460, AY835076, AY835104, AY835105, AY835106, AY835107, AY835108, AY835109, AY835110, AY835111, AY835112, AY835113, AY835114, AY835115, AY835116, AY835117, AY835118, AY835119, AY835120, AY835121, AY835122, AY835123, AY835124, AY835125, AY835126, AY835127, AY835128, AY835129, AY835130, AY835131, AY835132, AY835133, AY835134, AY835135, AY835136, AY835137, AY835138, AY835139, AY835140, AY835141, AY835142, AY835143, AY835144, AY835145, AY835146, AY835147, AY835148, AY835149, AY835150, AY835151, AY835152, AY835153, AY835154, AY835155, AY835156, AY835157, AY835158, AY835159, AY835160, AY835161, AY835162, AY835163, AY835164, AY835165, AY835166, AY835167, AY835168, AY835169, AY835170, AY835171, AY835172, AY835173, AY835174, AY835175, AY835176, AY835177, AY835178, AY835179, AY835180, AY835181, AY835182, AY835183, AY835184, AY835185, AY835186, AY835187, AY835188, AY835189, AY835190, AY835191, AY835192, AY835227, AY835228, AY835229, AY835230, AY835231, AY835232, AY835233, AY835234, AY835235, AY835236, AY835237, AY835238, AY835239, AY835240, AY835241, AY835242, AY835243, AY835244, AY835245, AY835246, AY835247, AY835248, AY835249, AY835250, AY835251, AY835252, AY835253, AY835254, AY835255, AY835256, AY835257, AY835258, AY835259, AY835260, AY835261, AY835262, AY835263, AY835264, AY835265, AY835266, AY835267, AY835268, AY835269, AY835270, AY835271, AY835272, AY835273, AY835274, AY835275, AY835276, AY835277, AY835278, AY835279, AY835280, AY835281, AY835282, AY835283, AY835284, AY835285, AY835286, AY835287, AY835288, AY835289, AY835290, AY835291, AY835292, AY835293, AY835294, AY835295, AY835296, AY835297, AY835298, AY835299, AY835300, AY835301, AY835302, AY835303, AY835304, AY835305, AY835306, AY835307, AY835308, AY835309, AY835310, AY835311, AY835312, AY835313, AY835314, AY835315, AY835316, AY835317, AY859526, AY878652, AY898811, AY898812, AY898813, AY898814, AY898815, AY898816, AY898817, AY898818, AY898819, AY898820, AY898821, AY898822, AY898823, AY898824, AY898825, AY898826, AY898827, AY898828, AY898829, AY898830, AY898831, AY898832, AY898833, AY898834, AY898835, AY898836, AY898837, AY898838, AY898839, AY898840, AY898841, AY898842, AY898843, AY898844, AY898845, AY898846, AY898847, AY898848, AY898849, AY898850, AY898851, AY898852, AY898853, AY898854, AY921165, AY921166, AY921167, AY921168, AY921169, AY921170, AY921171, AY921172, AY921608, AY940619, AY940620, AY940621, AY940622, D00574, D00689, D00691, D00832, D00944, D10074, D10687, D10688, D10750, D10934, D10988, D11168, D11355, D13406, D13558, D14116, D14484, D14829, D14830, D14831, D14853, D16694, D16695, D16697, D16698, D16702, D16703, D16704, D16705, D16722, D16723, D16738, D16739, D16740, D16741, D16742, D16748, D16749, D16750, D16751, D16752, D16753, D16754, D16755, D16763, D16764, D16765, D16766, D16767, D16768, D16769, D16770, D16805, D16806, D17763, D28918, D28919, D28920, D28921, D28922, D28923, D28924, D28925, D28926, D28927, D28928, D28929, D28930, D28931, D28932, D28933, D30613, D30614, D45172, D49374, D49455, D49462, D50409, D50480, D50481, D50482, D50483, D50484, D50485, D63821, D63822, D63857, D83645, D84262, D84263, D84264, D84265, D85516, D89815, D89872, D90208, DQ001223, DQ001224, DQ001225, DQ001226, DQ001227, DQ001228, DQ001229, DQ001230, DQ001231, DQ001232, DQ001233, DQ001234, DQ001235, DQ001236, DQ001237, DQ001238, DQ001239, DQ001240, DQ001241, DQ001242, DQ001243, DQ001244, DQ001245, DQ001246, DQ001247, DQ001248, DQ001249, DQ001267, DQ001268, DQ001270, DQ061296, DQ061297, DQ061298, DQ061299, DQ061331, DQ061332, DQ061333, DQ061334, DQ061335, DQ061336, DQ061337, DQ061338, DQ061339, DQ061340, DQ061341, DQ061342, DQ061343, DQ061344, DQ061345, DQ061346, DQ061347, DQ061348, DQ061349, DQ061350, DQ061351, DQ061352, DQ061353, DQ061354, DQ061355, DQ061356, DQ061357, DQ061358, DQ061359, DQ061360, DQ061361, DQ061362, DQ061363, DQ061364, DQ061365, DQ061366, DQ061367, DQ061368, DQ061369, DQ061370, DQ061371, DQ061372, DQ061373, DQ061374, DQ061375, DQ061376, DQ061377, DQ061378, DQ065829, DQ065836, DQ071885, DQ155449, DQ155454, DQ155462, DQ155466, DQ155471, DQ155473, DQ155485, DQ155486, DQ155488, DQ155489, DQ155495, DQ155497, DQ155498, DQ155561, DQ228488, DQ228489, DQ228490, DQ228491, DQ228492, DQ228493, DQ233304, DQ233305, DQ233306, DQ233307, DQ233308, DQ233309, DQ233310, DQ233311, DQ233312, DQ233313, DQ233314, DQ249537, DQ249538, DQ249539, DQ249540, DQ249541, DQ249542, DQ249543, DQ249544, DQ249545, DQ249546, DQ249547, DQ249548, DQ249549, DQ249550, DQ249551, DQ249552, DQ249553, DQ249554, DQ249555, DQ249556, DQ249557, DQ249558, DQ249559, DQ249560, DQ249561, DQ249562, DQ249563, DQ249564, DQ249565, DQ249566, DQ249567, DQ249568, DQ249569, DQ249570, DQ249571, DQ249623, DQ249624, DQ249625, DQ249626, DQ249627, DQ249628, DQ249629, DQ249630, DQ249631, DQ249632, DQ249633, DQ249634, DQ249635, DQ249636, DQ249637, DQ249638, DQ249639, DQ249640, DQ249641, DQ278891, DQ278892, DQ278893, DQ314805, DQ314806, DQ374420, DQ374421, DQ418786, DQ418788, DQ480513, DQ485285, DQ516083, DQ518404, DQ640336, DQ640350, DQ640354, DQ641950, DQ641951, DQ641952, DQ641953, DQ641954, DQ641955, DQ641956, DQ641957, DQ641958, DQ641959, DQ641960, DQ641961, DQ641962, DQ641963, DQ641964, DQ641965, DQ641966, DQ641967, DQ641968, DQ641969, DQ641970, DQ641971, DQ641972, DQ641973, DQ641974, DQ641975, DQ641976, DQ641977, DQ648495, DQ648496, DQ648497, DQ777787, DQ777788, DQ777789, DQ777790, DQ777791, DQ777792, DQ777793, DQ777794, DQ777795, DQ777796, DQ777806, DQ777807, DQ777808, DQ777809, DQ835760, DQ835761, DQ835762, DQ835763, DQ835764, DQ835765, DQ835766, DQ835769, DQ835770, DQ839181, DQ839182, DQ839183, DQ839184, DQ839185, DQ839186, DQ839187, DQ839188, DQ839189, DQ839190, DQ839191, DQ839192, DQ839193, DQ839194, DQ839195, DQ839196, DQ839197, DQ839198, DQ839199, DQ839200, DQ839201, DQ839202, DQ839203, DQ839204, DQ839205, DQ839206, DQ839207, DQ839208, DQ839209, DQ839210, DQ839211, DQ839212, DQ839213, DQ839214, DQ839215, DQ839216, DQ839217, DQ839218, DQ839219, DQ839220, DQ859926, DQ859927, DQ859928, DQ859929, DQ859933, DQ859934, DQ859935, DQ859936, DQ859939, DQ859945, DQ859947, DQ859951, DQ859961, DQ859967, EF032892, EF032893, EF032894, EF108306, EF175795, EF175798, EF205221, EF205229, EF407419, EF407458, EF407459, EF407460, EF407461, EF407462, EF407463, EF407464, EF407465, EF407466, EF407467, EF407468, EF407469, EF407470, EF407471, EF407472, EF407473, EF407474, EF407475, EF407476, EF407477, EF407478, EF407479, EF407480, EF407481, EF407482, EF407483, EF407484, EF407485, EF407486, EF407487, EF407488, EF407489, EF407490, EF407491, EF407492, EF407493, EF407494, EF407495, EF407496, EF407497, EF407498, EF407499, EF407500, EF407501, EF407502, EF407503, EF407504, EF420126, EF420127, EF424625, EF424626, EF424627, EF424628, EF424629, EF543226, EF543227, EF543228, EF543229, EF543230, EF543231, EF543232, EF543234, EF543235, EF543236, EF543237, EF543238, EF560531, EF560532, EF560533, EF560534, EF560551, EF560552, EF560553, EF560554, EF589160, EF589161, EF632069, EF632070, EF632071, EF638081, EF652512, EF652513, EF652514, EF652515, EF652516, EF652517, EF652518, EF652519, EF652520, EF652521, EF652522, EF652523, EF652524, EF652525, EF652526, EF652527, EF652528, EF652529, EF652530, EF652531, EF652532, EF652533, EF652534, EF652535, EF652536, EF652537, EF652538, EF652539, EF652540, EF652541, EF652542, EF652543, EF652544, EF652545, EF652546, EF652547, EF652548, EF652549, EF652550, EF652551, EF652552, EF652553, EF652554, EF652555, EF652556, EF652557, EF652558, EF652559, EF652560, EF652561, EF652562, EF652563, EF652564, EF652565, EF652566, EF652567, EF652568, EF652569, EF652570, EF652571, EF652572, EF652573, EF652574, EF652575, EF652576, EF652577, EF652578, EF652579, EF652580, EF652581, EF652582, EF652583, EF652584, EF652585, EF652586, EF652587, EF652588, EF652589, EF652590, EF652591, EF652592, EF652593, EF652594, EF652595, EF652596, EF652597, EF652598, EF652599, EF652600, EF652601, EF652602, EF652603, EF652604, EF652605, EF652606, EF652607, EF652608, EF652609, EF652610, EF652611, EF652612, EF652613, EF652614, EF652615, EF652616, EF652617, EF652618, EF652619, EF652620, EF652621, EF652622, EF652623, EF652624, EF652625, EF652626, EF652627, EF652628, EF652629, EF652630, EF652631, EF652632, EF652633, EF652634, EF652635, EF652636, EF652637, EF652638, EF652639, EF652640, EF652641, EF652642, EF652643, EF652644, EF652645, EF652646, EF652647, EF652648, EF652649, EF652650, EF652651, EF652652, EF652653, EF652654, EF652655, EF652656, EF652657, EF652658, EF652659, EF652660, EF652661, EF652662, EF652663, EF652664, EF652665, EF652666, EF652667, EF652668, EF652669, EF652670, EF652671, EF652672, EF652673, EF652674, EF652675, EF652676, EF652677, EF652678, EF652679, EF652680, EF652681, EF652682, EF652683, EF652684, EF652685, EF652686, EF652687, EF652688, EF652689, EF652690, EF652691, EF652692, EF652693, EF652694, EF652695, EF652696, EF652697, EF652698, EF652699, EF652700, EF652701, EF652702, EF652703, EF652704, EF652705, EF652706, EF652707, EF652708, EF652709, EF652710, EF652711, EF652712, EF652713, EF652714, EF652715, EF652716, EF652717, EF652718, EF652719, EF652720, EF652721, EF652722, EF652723, EF652724, EF652725, EF652726, EF652727, EF652728, EF652729, EF652730, EF652731, EF652732, EF652733, EF652734, EF652735, EF652736, EF652737, EF652738, EF652739, EF652740, EF652741, EF652742, EF652743, EF652744, EF652745, EF652746, EF652747, EF652748, EF652749, EF652750, EF652751, EF652752, EF652753, EF652754, EF652755, EF652756, EF652757, EF652758, EF652759, EF652760, EF652761, EF652762, EF652763, EF652764, EF652765, EF652766, EF652767, EF652768, EF652769, EF652770, EF652771, EF652772, EF652773, EF652774, EF652775, EF652776, EF652777, EF652778, EF652779, EF652780, EF652781, EF652782, EF652783, EF652784, EF652785, EF652786, EF652787, EF652788, EF652789, EF652790, EF652791, EF652792, EF652793, EF652794, EF652795, EF652796, EF652797, EF652798, EF652799, EF652800, EF652801, EF652802, EF652803, EF652804, EF652805, EF652806, EF652807, EF652808, EF652809, EF652810, EU081313, EU081375, EU081379, EU081380, EU081382, EU081383, EU081388, EU081394, EU081401, EU081407, EU081409, EU081410, EU081412, EU081421, EU081423, EU081424, EU155217, EU155218, EU155219, EU155220, EU155221, EU155222, EU155223, EU155224, EU155225, EU155226, EU155227, EU155228, EU155229, EU155230, EU155231, EU155232, EU155234, EU155235, EU155253, EU155254, EU155255, EU155256, EU155257, EU155258, EU155259, EU155260, EU155261, EU155262, EU155263, EU155264, EU155279, EU155280, EU155281, EU155300, EU155301, EU155302, EU155303, EU155304, EU155305, EU155306, EU155307, EU155308, EU155315, EU155316, EU155317, EU155318, EU155324, EU155325, EU155326, EU155327, EU155328, EU155329, EU155330, EU155331, EU155332, EU155333, EU155334, EU155335, EU155336, EU155337, EU155356, EU155357, EU155358, EU155359, EU155360, EU155361, EU155362, EU155363, EU155364, EU155365, EU155366, EU155367, EU155368, EU155369, EU155370, EU155371, EU155372, EU155373, EU155374, EU155375, EU155376, EU155377, EU155381, EU155382, EU158186, EU233421, EU233422, EU233423, EU233425, EU233426, EU233427, EU234061, EU234062, EU239714, EU246931, EU246932, EU246933, EU246934, EU246935, EU246936, EU246937, EU246938, EU246939, EU246940, EU255960, EU255961, EU255962, EU256000, EU256001, EU256045, EU256054, EU256059, EU256061, EU256062, EU256064, EU256065, EU256066, EU256075, EU256076, EU256077, EU256078, EU256079, EU256080, EU256081, EU256082, EU256083, EU256084, EU256085, EU256088, EU256089, EU256090, EU256091, EU256092, EU256098, EU256099, EU256100, EU256101, EU256102, EU256103, EU257482, EU257483, EU333897, EU333898, EU333899, EU392169, EU392170, EU392171, EU392172, EU392173, EU392174, EU392175, EU408326, EU408327, EU408328, EU408329, EU408330, EU408331, EU408332, EU414144, EU414145, EU414146, EU414147, EU414148, EU414149, EU414150, EU414151, EU414152, EU414153, EU414154, EU414155, EU414156, EU414157, EU414158, EU414159, EU414160, EU414161, EU414162, EU482833, EU482839, EU482849, EU482859, EU482860, EU482874, EU482875, EU482877, EU482879, EU482880, EU482881, EU482883, EU482885, EU482886, EU482888, EU529682, EU589464, EU589465, EU589466, EU589467, EU589468, EU589469, EU589470, EU589471, EU589472, EU589473, EU589474, EU643834, EU643836, EU660386, EU660388, EU781825, EU781826, EU781827, EU781828, EU781829, EU781830, EU781831, EU781832, EU798760, EU798761, EU857431, EU862835, EU862837, FJ024086, FJ024277, FJ024279, FJ025854, FJ025855, FJ025856, FJ390396, FJ390397, FJ390398, FJ407092, FJ411253, FJ411254, FJ411255, FJ411256, FJ435090, FJ462431, FJ462432, FJ462433, FJ462434, FJ462435, FJ462436, FJ462437, FJ462438, FJ462439, FJ462440, FJ462441, FJ477240, FJ478453, FJ483361, FJ483362, FJ483363, FJ483364, FJ483365, FJ483366, FJ483367, FJ483368, FJ483369, FJ483370, FJ483371, FJ483372, FJ483373, FJ483374, FJ483375, FJ483376, FJ483377, FJ483378, FJ483379, FJ483380, FJ483381, FJ483382, FJ483383, FJ483384, FJ483385, FJ483386, FJ483387, FJ483388, FJ483389, FJ483390, FJ483391, FJ483392, FJ483393, FJ483394, FJ483395, FJ483396, FJ483397, FJ483398, FJ483399, FJ483400, FJ483401, FJ483402, FJ483403, FJ483404, FJ483405, FJ483406, FJ483407, FJ597156, FJ597157, FJ597158, FJ597164, FJ607065, FJ607066, FJ607067, FJ607068, FJ607069, FJ607070, FJ607071, FJ607072, FJ607073, FJ607074, FJ607075, FJ607076, FJ607077, FJ607078, FJ607079, FJ607080, FJ607081, FJ607082, FJ607083, FJ607084, FJ607085, FJ607086, FJ607087, FJ607088, FJ607089, FJ607090, FJ607091, FJ607092, FJ607093, FJ607094, FJ607095, FJ607096, FJ607097, FJ607098, FJ607099, FJ607100, FJ607101, FJ607102, FJ607103, FJ607104, FJ607105, FJ607106, FJ607107, FJ607108, FJ607109, FJ607110, FJ607111, FJ607112, FJ607113, FJ607114, FJ607115, FJ607116, FJ607117, FJ607118, FJ607119, FJ607120, FJ607121, FJ607122, FJ607123, FJ607124, FJ607125, FJ607126, FJ607127, FJ607128, FJ607129, FJ607130, FJ607131, FJ607132, FJ607133, FJ768870, FJ768871, FJ768872, FJ768873, FJ768874, FJ768875, FJ768876, FJ768877, FJ768878, FJ768879, FJ768880, FJ768881, FJ839869, FJ839870, FJ911706, FJ911715, FJ911717, FJ911733, FJ948016, FJ948017, FJ948018, FJ948031, FJ948034, FJ948036, FJ948045, FJ948046, FJ948047, FJ948048, FN435993, FN665803, FN666283, FN666284, FN666285, FN666286, FN666287, FN666288, FN666289, FN666325, FN666327, FN666428, FN666429, FN675941, FN675942, FN675943, FN675944, FN675945, FN675946, FN675947, FN675948, FN675949, FN675950, FN675951, FN675952, FN675953, FN675954, FN675955, FN675956, FN675957, FN675958, FN675959, FN675960, FN675961, FN675962, FN675963, FN675964, FN675965, FN675966, FN675967, FN675968, FN675969, FN675970, FN675971, FN675972, FN675973, FN675974, FN675975, FN675976, FN675977, FN675978, FN675979, FN675980, FN675981, FN675982, FN675983, FR850665, FR850667, FR850669, FR850672, FR850673, FR850674, GQ205675, GQ205676, GQ205677, GQ205678, GQ205679, GQ205680, GQ205681, GQ205682, GQ205683, GQ205684, GQ205685, GQ205686, GQ205687, GQ205688, GQ205689, GQ205690, GQ205691, GQ205692, GQ205693, GQ205694, GQ205695, GQ205696, GQ205697, GQ205698, GQ205699, GQ205700, GQ205701, GQ205702, GQ205703, GQ205704, GQ205705, GQ205706, GQ205707, GQ205708, GQ205709, GQ205710, GQ205711, GQ205712, GQ205713, GQ205714, GQ205715, GQ205716, GQ205717, GQ205718, GQ205719, GQ205720, GQ205721, GQ205722, GQ205723, GQ205724, GQ205725, GQ205726, GQ205727, GQ205728, GQ205729, GQ205730, GQ205731, GQ205732, GQ205733, GQ205734, GQ205735, GQ205736, GQ205737, GQ205738, GQ205739, GQ205740, GQ205741, GQ205742, GQ205743, GQ205744, GQ205745, GQ205746, GQ205747, GQ205748, GQ205749, GQ205750, GQ205751, GQ205752, GQ205753, GQ205754, GQ205755, GQ205756, GQ205757, GQ205758, GQ205759, GQ244326, GQ244328, GQ244329, GQ244330, GQ244331, GQ247874, GQ247875, GQ247876, GQ247877, GQ292447, GQ292448, GQ292449, GQ292450, GQ292451, GQ913858, GQ913859, GQ913860, GQ913861, GQ913862, GQ913863, GQ913864, GU133617, GU186927, GU186929, GU186932, GU186952, GU186953, GU186959, GU186963, GU289921, GU441243, GU441244, GU441245, GU441246, GU441247, GU441248, GU441249, GU441250, GU441251, GU441252, GU441253, GU441254, GU441255, GU441256, GU441257, GU441259, GU441260, GU441261, GU441262, GU441263, GU441264, GU441265, GU441266, GU441267, GU441268, GU441269, GU441270, GU441271, GU441272, GU441273, GU441274, GU441275, GU441276, GU441277, GU441278, GU441279, GU441280, GU441281, GU441282, GU441283, GU441284, GU441285, GU441286, GU441287, GU441288, GU441289, GU441290, GU441291, GU441292, GU441293, GU441294, GU441295, GU441296, GU441297, GU441298, GU441299, GU441300, GU441301, GU441302, GU441303, GU441304, GU441305, GU441306, GU441307, GU441308, GU441309, GU441310, GU441311, GU441312, GU441385, GU451218, GU451219, GU451220, GU451221, GU451222, GU451223, GU451224, HM041985, HM041986, HM041987, HM041988, HM041989, HM041990, HM041991, HM041992, HM041993, HM041994, HM041995, HM041996, HM041997, HM041998, HM041999, HM053611, HM106522, HM106523, HM106524, HM106525, HM106526, HM106527, HM106528, HM106529, HM106530, HM106531, HM106532, HM106533, HM106534, HM106535, HM106536, HM106537, HM106538, HM106539, HM106540, HM106541, HM106542, HM106543, HM106544, HM106545, HM106546, HM106547, HM106548, HM106549, HM106550, HM106551, HM106552, HM106553, HM106554, HM106555, HM106556, HM106557, HM106558, HM106559, HM106560, HM106561, HM106562, HM106563, HM106564, HM106565, HM106566, HM106567, HM106568, HM161675, HM777358, HM777359, HM777361, HM777366, HM777370, HM777377, HM777402, HM777405, HM777406, HM777409, HM777410, HM777411, HM777413, HM777414, HM777415, HM777418, HM777421, HM777424, HM777425, HM777426, HQ110091, HQ113696, HQ113697, HQ113698, HQ113699, HQ113700, HQ113701, HQ229038, HQ229043, HQ229046, HQ229048, HQ229054, HQ229061, HQ229062, HQ229088, HQ229090, HQ229095, HQ229101, HQ229109, HQ229110, HQ229112, HQ229114, HQ229119, HQ229125, HQ229128, HQ229129, HQ229130, HQ229131, HQ229135, HQ229143, HQ229149, HQ229156, HQ229157, HQ229158, HQ229168, HQ229171, HQ229173, HQ229176, HQ229178, HQ229192, HQ229194, HQ229207, HQ229209, HQ229220, HQ229226, HQ229235, HQ229241, HQ229246, HQ229250, HQ229255, HQ229257, HQ229260, HQ229262, HQ537008, HQ537009, HQ639937, HQ639940, HQ639946, HQ639947, HQ719473, HQ729726, HQ729727, HQ729728, HQ729729, HQ729730, HQ729736, HQ729737, HQ729739, HQ729755, HQ729756, HQ729757, HQ729758, HQ729759, HQ912956, HQ912957, HQ912958, HQ912959, JF721245, JF721246, JF721247, JF721248, JF721249, JF721250, JF721251, JF721252, JF721253, JF721254, JF721255, JF721256, JF721257, JF721258, JF824180, JF824188, JF824219, JF824229, JF824237, JF824303, JF824317, JN055400, JN055401, JN055403, JN055406, JN055407, JN055409, JN055410, JN055411, JN055412, JN055413, JN055414, JN055416, JN055417, JN055418, JN055420, JN055421, JN055422, JN055423, JN055429, JN055431, JN055432, JN18490, JN120912, JN214605, JN214611, JN214619, JN214625, JN214630, JN214643, JN214648, JN214661, JN214676, JN214677, JN214681, JN214684, JN214685, JN214689, JN214692, JN214695, JN214697, JN214699, JN214706, JN214710, JN214714, JN214718, JN214727, JN214732, JN214739, JN214763, JN214778, JN214790, JN214795, JN214806, JN214807, JN214812, JN214813, JN214826, JN214837, JN214838, JN214843, JN214848, JN214859, JN214867, JN214882, JN214884, JN573003, JN573005, JN573006, JN573007, JN573008, JN573009, JN573010, JN573011, JN573012, JN573013, JN573018, JN573020, JN573021, JN573023, JN573024, JN573025, JN573026, JN573030, JN573031, JN573032, JN573036, JN573037, JN573039, JN573042, JN573045, JN573047, JN573049, JN573050, JN573052, JN573054, JN573055, JN573056, JN573057, JN573058, JN573061, JN573062, JN573063, JN573064, JN573065, JN573066, JN573067, JN573069, JN573070, JN573071, JN573072, JN573073, JN573075, JN573077, JN573078, JN573080, JN573084, JN573086, JN573087, JN573089, JN573090, JN573091, JN573092, JN573093, JN573094, JN573095, JN573098, JN870282, JN981168, JN981169, JN981170, JN981171, JN981172, JN981173, JN981174, JN981175, JN981176, JN981177, JN981178, JN981179, JN981180, JN981181, JN981182, JN981183, JN981184, JN981185, JN981186, JN981187, JN981188, JN981189, JN981190, JN981191, JN981192, JN981193, JN981194, JN981195, JN981196, JN981197, JN981198, JN981199, JN981200, JN981201, JN981202, JN981203, JN981204, JN981205, JN981206, JN981207, JN981208, JN981209, JN981210, JN981211, JN981212, JN981213, JN981214, JN981215, JN981216, JN981217, JN981218, JN981219, JN981220, JN981221, JN981222, JN981223, JN981224, JN981225, JN981226, JN981227, JN981228, JN981229, JN981230, JN981231, JN981232, JN981233, JN981234, JN981235, JN981236, JN981237, JN981238, JN981239, JN981240, JN981241, JN981242, JN981243, JN981244, JN981245, JN981246, JN981247, JN981248, JN981249, JN981250, JN981251, JN981252, JN981253, JN981254, JN981255, JN981256, JN981257, JN981258, JN981259, JN981260, JN981261, JN981262, JN981263, JN981264, JN981265, JN981266, JN981267, JN981268, JN981269, JN981270, JN981271, JN981272, JN981273, JN981274, JN981275, JN981276, JN981277, JN981278, JN981279, JN981280, JN981281, JN981282, JN981283, JN981284, JN981285, JN981286, JN981287, JN981288, JN981289, JN981290, JN981291, JN981292, JN981293, JN981294, JN981295, JN981296, JN981297, JN981298, JN981299, JN981300, JN981301, JN981302, JN981303, JN981304, JN981305, JN981306, JN981307, JN981308, JN981309, JN981310, JN981311, JN981312, JN981313, JN981314, JN981315, JN981316, JN981317, JN981318, JN981319, JN981320, JN981321, JN981322, JN981323, JN981324, JN981325, JN981326, JN981327, JN981328, JN981329, JN981330, JN981331, JN981332, JN981333, JN981334, JN981335, JN981336, JN981337, JN981338, JN981339, JN981340, JN981341, JN981342, JN981343, JN981344, JN981345, JN981346, JN981347, JN981348, JN981349, JN981350, JN981351, JN981352, JN981353, JN981354, JN981355, JN981356, JN981357, JN981358, JN981359, JN981360, JN981361, JN981362, JN981363, JN981364, JN981365, JN981366, JN981367, JN981368, JN981369, JN981370, JN981371, JN981372, JN981373, JN981374, JN981375, JN981376, JN981377, JN981378, JN981379, JN981380, JN981381, JN981382, JN981383, JN981384, JN981385, JN981386, JN981387, JN981388, JN981389, JN981390, JN981391, JN981392, JN981393, JN981394, JN981395, JN981396, JN981397, JN981398, JN981399, JN981400, JN981401, JN981402, JN981403, JN981404, JN981405, JN981406, JN981407, JN981408, JN981409, JN981410, JN981411, JN981412, JN981413, JN981414, JN981415, JN981416, JN981417, JN981418, JN981419, JN981420, JN981421, JN981422, JN981423, JN981424, JN981425, JN981426, JN981427, JN981428, JN981429, JN981430, JN981431, JN981432, JN981433, JN981434, JN981435, JN981436, JN981437, JN981438, JN981439, JN981440, JN981441, JN981442, JN981443, JN981444, JN981445, JN981446, JN981447, JN981448, JN981449, JN981450, JN981451, JN981452, JN981453, JN981454, JN981455, JN981456, JN981457, JN981458, JN981459, JN981460, JN981461, JN981462, JN981463, JN981464, JN981465, JN981466, JN981467, JN981468, JN981469, JN981470, JN981471, JN981472, JN981473, JN981474, JN981475, JN981476, JN981477, JN981478, JN981479, JN981480, JN981481, JN981482, JN981483, JN981484, JN981485, JN981486, JN981487, JN981488, JN981489, JN981490, JN981491, JN981492, JN981493, JN981494, JN981495, JN981496, JN981497, JN981498, JN981499, JN981500, JN981501, JN981502, JN981503, JN981504, JN981505, JN981506, JQ253518, JQ253519, JQ253520, JQ253521, JQ253522, JQ253523, JQ253524, JQ253525, JQ253526, JQ253527, JQ253528, JQ253529, JQ253530, JQ253531, JQ253532, JQ253533, JQ253534, JQ253535, JQ253536, JQ253537, JQ253538, JQ253539, JQ253540, JQ253541, JQ668313, JQ668314, JQ791266, JQ791267, JQ791268, JQ791269, JQ791270, JQ791271, JQ791272, JQ791273, JQ791274, JQ791275, JQ791276, JQ791277, JQ791278, JQ791279, JQ791280, JQ791281, JQ791282, JQ791283, JQ791284, JQ791285, JQ791286, JQ791287, JQ791288, JQ791289, JQ791290, JQ791291, JQ791292, JQ791293, JQ791294, JQ791295, JQ791296, JQ791297, JQ791298, JQ791299, JQ791300, JQ791301, JQ791302, JQ791303, JQ791304, JQ791305, JQ791306, JQ791307, JQ791308, JQ791309, JQ791310, JQ791311, JQ791312, JQ791313, JQ791314, JQ791315, JQ791316, JQ791317, JQ791318, JQ791319, JQ791320, JQ791321, JQ791322, JQ791323, JQ791324, JQ791325, JQ791326, JQ791327, JQ791328, JQ791329, JQ791330, JQ791331, JQ791332, JQ791333, JQ791334, JQ791335, JQ791336, JQ791337, JQ791338, JQ791339, JQ791340, JQ791341, JQ791342, JQ791343, JQ791344, JQ791345, JQ791346, JQ791347, JQ791348, JQ791349, JQ791350, JQ791351, JQ791352, JQ791353, JQ791354, JQ791355, JQ791356, JQ791357, JQ791358, JQ791359, JQ791360, JQ791361, JQ791362, JQ791363, JQ791364, JQ791365, JQ791366, JQ791367, JQ791368, JQ791369, JQ791370, JQ791371, JQ791372, JQ791373, JQ791374, JQ791375, JQ791376, JQ791377, JQ791378, JQ791379, JQ791380, JQ791381, JQ791382, JQ791383, JQ791384, JQ791385, JQ791386, JQ791387, JQ791388, JQ791389, JQ791390, JQ791391, JQ791392, JQ791393, JQ791394, JQ791395, JQ791396, JQ791397, JQ791398, JQ791399, JQ791400, JQ791401, JQ791402, JQ791403, JQ791404, JQ801813, JQ801814, JQ801815, JQ801816, JQ801817, JQ801818, JQ801819, JQ801820, JQ801821, JQ801822, JQ801823, JQ801824, JQ801825, JQ801826, JQ801827, JQ801828, JQ801829, JQ801830, JQ801831, JQ801832, JQ801833, JQ801834, JQ801835, JQ801836, JQ801837, JQ801838, JQ801839, JQ801840, JQ801841, JQ801842, JQ801843, JQ801844, JQ801845, JQ801846, JQ801847, JQ801848, JQ801849, JQ801850, JQ801851, JQ801852, JQ801922, JQ801923, JQ801924, JQ801925, JQ801926, JQ801927, JQ801928, JQ801929, JQ801930, JQ801931, JQ801932, JQ801933, JQ801934, JQ801935, JQ801936, JQ801937, JQ801938, JQ801939, JQ801940, JQ801941, JQ801942, JQ801943, JQ801944, JQ801945, JQ801946, JQ801947, JQ801948, JQ802087, JQ802088, JQ802089, JQ802090, JQ802091, JQ802092, JQ802093, JQ802094, JQ802095, JQ802096, JQ802097, JQ802098, JQ802099, JQ802100, JQ802101, JQ802104, JQ802105, JQ803005, JQ803006, JQ803007, JQ803008, JQ803009, JQ803010, JQ803011, JQ803012, JQ803013, JQ803014, JQ803015, JQ803016, JQ803017, JQ803018, JQ803019, JQ803020, JQ803021, JQ803022, JQ803023, JQ803024, JQ803025, JQ803026, JQ803027, JQ803028, JQ803029, JQ803030, JQ803031, JQ803032, JQ803033, JQ803034, JQ803035, JQ803036, JQ803037, JQ803038, JQ803039, JQ803040, JQ803041, JQ803042, JQ803043, JQ803044, JQ803045, JQ803046, JQ803047, JQ803048, JQ803049, JQ803050, JQ803051, JQ803052, JQ803053, JQ803054, JQ803055, JQ803056, JQ803057, JQ803058, JQ803059, JQ803060, JQ803061, JQ803062, JQ803063, JQ803064, JQ803065, JQ803066, JQ803067, JQ803068, JQ803069, JQ803070, JQ803071, JQ803072, JQ803073, JQ803074, JQ803075, JQ803076, JQ803077, JQ803078, JQ803079, JQ803080, JQ803081, JQ803082, JQ803083, JQ803084, JQ803085, JQ803086, JQ803087, JQ803088, JQ803089, JQ803090, JQ803091, JQ803092, JQ803093, JQ803094, JQ803095, JQ803096, JQ803097, JQ803098, JQ803099, JQ803100, JQ803101, JQ803102, JQ803103, JQ803104, JQ803105, JQ803106, JQ803107, JQ803108, JQ803109, JQ803110, JQ803111, JQ803112, JQ803113, JQ803114, JQ803115, JQ803116, JQ803117, JQ803118, JQ803119, JQ803120, JQ803121, JQ803122, JQ803585, JQ803586, JQ803587, JQ803588, JQ803589, JQ803590, JQ803591, JQ804029, JQ804030, JQ804031, JQ804032, JQ804033, JQ804034, JQ804035, JQ804036, JQ804037, JQ804038, JQ804039, JQ804040, JQ804041, JQ804042, JQ804043, JQ804044, JQ804045, JQ804046, JQ804047, JQ804048, JQ804049, JQ804050, JQ804051, JQ804052, JQ804053, JQ804054, JQ804055, JQ804056, JQ804057, JQ804058, JQ804059, JQ804060, JQ804061, JQ804062, JQ804063, JQ804064, JQ804065, JQ804066, JQ804067, JQ804068, JQ804069, JQ804070, JQ804071, JQ804072, JQ804073, JQ804074, JQ804075, JQ804076, JQ804077, JQ804078, JQ804079, JQ804080, JQ804081, JQ804082, JQ804083, JQ804084, JQ804085, JQ804086, JQ804087, JQ804088, JQ804089, JQ804090, JQ804091, JQ804092, JQ804093, JQ804094, JQ804095, JQ804096, JQ804097, JQ804098, JQ804099, JQ804100, JQ804101, JQ804102, JQ804103, JQ804104, JQ804105, JQ804106, JQ804107, JQ804108, JQ804109, JQ804110, JQ804111, JQ804112, JQ804113, JQ804114, JQ804115, JQ804116, JQ804117, JQ804118, JQ804119, JQ804120, JQ804121, JQ804122, JQ804123, JQ804124, JQ804125, JQ804126, JQ804127, JQ804128, JQ888223, JQ888225, JQ924889, JQ924890, JQ924891, JQ924892, JQ924893, JQ924894, JQ924895, JQ924896, JQ924897, JQ924898, JQ924899, JQ924900, JQ924901, JQ924902, JQ924903, JQ924904, JQ924905, JQ924906, JQ924907, JQ924908, JQ924909, JQ924910, JQ924911, JQ924912, JQ924913, JQ924914, JX183307, JX183308, JX183309, JX183310, JX183311, JX183357, JX183358, JX183359, JX183360, JX183361, JX183362, JX649674, JX649675, JX649676, JX649677, JX649678, JX649679, JX649680, JX649681, JX649682, JX649683, JX649684, JX649685, JX649686, JX649687, JX649688, JX649689, JX649690, JX649691, JX649692, JX649693, JX649694, JX649695, JX649696, JX649697, JX649698, JX649699, JX649700, JX649701, JX649702, JX649703, JX649704, JX649705, JX649706, JX649707, JX649708, JX649709, JX649710, JX649711, JX649712, JX649713, JX649714, JX649715, JX649716, JX649717, JX649718, JX649719, JX649720, JX649721, JX649722, JX649723, JX649724, JX649725, JX649726, JX649727, JX649728, JX649729, JX649730, JX649731, JX649732, JX649733, JX649734, JX649735, JX649736, JX649737, JX649738, JX649739, JX649740, JX649741, JX649742, JX649743, JX649744, JX649745, JX649746, JX649747, JX649748, JX649749, JX649750, JX649751, JX649752, JX649753, JX649754, JX649755, JX649756, JX649757, JX649758, JX649759, JX649760, JX649761, JX649762, JX649763, JX649764, JX649765, JX649766, JX649767, JX649768, JX649769, JX649770, JX649771, JX649772, JX649773, JX649774, JX649775, JX649776, JX649777, JX649778, JX649779, JX649780, JX649781, JX649782, JX649783, JX649784, JX649785, JX649786, JX649787, JX649788, JX649789, JX649790, JX649791, JX649792, JX649793, JX649794, JX649795, JX649796, JX649797, JX649798, JX649799, JX649800, JX649801, JX649802, JX649803, JX649804, JX649805, JX649806, JX649807, JX649808, JX649809, JX649810, JX649811, JX649812, JX649813, JX649814, JX649815, JX649816, JX649817, JX649818, JX649819, JX649820, JX649821, JX649822, JX649823, JX649824, JX649825, JX649826, JX649827, JX649828, JX649829, JX649830, JX649831, JX649832, JX649833, JX649834, JX649835, JX649836, JX649837, JX649838, JX649839, JX649840, JX649841, JX649842, JX649843, JX649844, JX649845, JX649846, JX649847, JX649848, JX649849, JX649850, JX649851, JX649852, JX649853, JX676777, JX676778, JX676780, JX676781, JX676782, JX676783, JX676785, JX676786, JX676788, JX676789, JX676791, JX676792, JX676794, JX676795, JX676798, JX676800, JX676801, JX676802, JX676803, JX676804, JX676805, JX676806, JX676807, JX676808, JX676809, JX676810, JX676813, JX676815, JX676816, JX676817, JX676818, JX676820, JX676821, JX676823, JX676824, JX676825, JX676826, JX676828, JX676829, JX676831, JX676832, JX676834, JX676835, JX676837, JX676839, JX676840, JX676841, JX676842, JX676843, JX676845, JX676846, JX676847, JX676848, JX676849, JX676851, JX676852, JX676854, JX676856, JX676859, JX676861, JX676862, JX676864, JX676866, JX676869, JX676870, JX676871, JX676872, JX676874, JX676875, JX676877, JX676880, JX676881, JX676883, JX676884, JX676885, JX676886, JX676887, JX676888, JX676891, JX676894, JX676895, JX676896, JX676897, JX676899, JX676900, JX676901, JX676902, JX676903, JX676904, JX676905, JX676906, JX676907, JX676908, JX676909, JX676910, JX676914, JX676915, JX676916, JX676919, JX676920, JX676921, JX676922, JX676923, JX676925, JX676926, JX676928, JX676930, JX676931, JX676932, JX676933, JX676934, JX676935, JX676939, JX676940, JX676941, JX676942, JX676943, JX676944, JX676945, JX676947, JX676950, JX676953, JX676955, JX676956, JX676958, JX676959, JX676960, JX676963, JX676964, JX676966, JX676967, JX676969, JX676970, JX676971, JX676973, JX676974, JX676976, JX676977, JX676978, JX676979, JX676980, JX676981, JX676982, JX676984, JX676985, JX676986, JX676988, JX676989, JX676990, JX676991, JX676996, JX676997, JX676999, JX677000, JX677001, JX677003, JX677004, JX677006, JX677009, JX677010, JX677012, JX677013, JX677014, JX677015, JX677018, JX677019, JX677020, JX677025, JX677026, JX677027, JX677028, JX677029, JX677030, JX677035, JX677038, JX677039, JX677040, JX677041, JX677043, JX677044, JX677046, JX677047, JX677049, JX677050, JX677052, JX677053, JX677054, JX677055, JX677056, JX677058, JX677059, JX677062, JX677063, JX677066, JX677067, JX677068, JX677069, JX677072, JX677073, JX677075, JX677076, JX677077, JX677081, JX677082, JX677083, JX677084, JX677086, JX677088, JX677090, JX677092, JX677093, JX677094, JX677096, JX677097, JX677098, JX677100, JX677103, JX677105, JX677107, JX677108, JX677109, JX677110, JX677112, JX677113, JX677114, JX677115, JX677120, JX677121, JX677123, JX677124, JX677125, JX677126, JX677129, JX677131, JX677132, JX677133, JX677134, JX677135, JX677136, JX677137, JX677138, JX677139, JX677141, JX677144, JX677145, JX677146, JX677147, JX677148, JX677150, JX677151, JX677152, JX677154, JX677156, JX677157, JX677159, JX677160, JX677161, JX677163, JX677166, JX944427, JX944428, JX944429, JX944430, JX944431, JX944432, JX944433, JX944434, JX944435, KC18292, KC118293, KC118294, KC118295, KC118296, KC118297, KC118298, KC118299, KC118300, KC118301, KC118302, KC118303, KC118304, KC118305, KC118306, KC18307, KC118308, KC18309, KC143880, KC143881, KC143882, KC143883, KC143884, KC143885, KC143886, KC143887, KC143888, KC143889, KC143890, KC143891, KC143892, KC143893, KC143894, KC143895, KC143922, KC143923, KC143924, KC143925, KC143926, KC143927, KC143928, KC143929, KC143930, KC143931, KC285356, KC348431, KC348432, KC348433, KC348434, KC439481, KC439482, KC439483, KC439484, KC439485, KC439486, KC439487, KC439488, KC439489, KC439490, KC439491, KC439492, KC439493, KC439494, KC439495, KC439496, KC439497, KC439498, KC439499, KC439500, KC439501, KC439502, KC439503, KC439504, KC439505, KC439506, KC439507, KC439508, KC439509, KC439510, KC439511, KC439512, KC439513, KC439514, KC439515, KC439516, KC439517, KC439518, KC439519, KC439520, KC439521, KC439522, KC439523, KC439524, KC439525, KC439526, KC439527, KC844051, KC844052, KF181661, KF181662, KF181663, KF181664, KF181665, KF181666, KF181667, KF181668, KF181669, KF181670, KF181671, KF273114, KF273115, KF273116, KF273117, KF273121, KF586319, KF586320, KF586321, KF586322, KF586323, KF586324, KF586325, KF586326, KF586327, KF586328, KF586329, KF586330, KF586331, KF728598, KF728600, KF728601, L02836, L12354, L20498, L38318, L38343, L38344, L38345, L38346, L38348, L38351, L38421, L44598, L44599, L50543, L50555, M58335, M67463, M74806, M74807, M74809, M74810, M74813, M74814, M74815, M74888, M84754, M86765, M86779, M96362, NC_004102, NC_009826, S46012, S62220, S67463, S72727, S72728, S76540, S78528, S83169, U01214, U10189, U10193, U10199, U10200, U10201, U10202, U10203, U10204, U10205, U10209, U10212, U10213, U10223, U10225, U10227, U10234, U16362, U23385, U23389, U23390, U23744, U28029, U31232, U37592, U37593, U37598, U37599, U37600, U37601, U37602, U37603, U37604, U37605, U37606, U37607, U37608, U37609, U45461, U45462, U45463, U45464, U45465, U45466, U45467, U45468, U45469, U45470, U45471, U45472, U45473, U45474, U45475, U45476, U55282, U55284, U63376, U63377, U63378, U63379, U89015, U89019, U94722, X61591, X61592, X61593, X61594, X61595, X61596, X65924, X76408, X76409, X76918, X78950, X78951, X91297, X91302, X91304, X91305, Y11604, Y12083, Z29445, Z29446, Z29450, Z29451, Z29452, Z29453, and Z29454.

In some embodiments, an HCV core antigen polypeptide shares about 30%, about 35%, about 40%, about 45%, about 50%, about 55%, about 60%, about 65%, about 70%, about 75%, about 80%, about 85%, about 90%, about 99%, or 100% similarity with the amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, or SEQ ID NO: 11, or a fragment thereof. Thus, encompassed by the present disclosure are analogs of HCV core antigen polypeptides (nucleic or amino acid) that have altered functional (e.g., immunogenic) properties relative to the starting fragment.

As discussed herein, embodiments of an HCV core antigen polypeptide disclosed herein include polypeptides containing less than the full amino acid sequence of SEQ ID NO: 1. For example, representative embodiments of an HCV core antigen polypeptide disclosed herein comprise peptides/proteins having any 4, 5, 6, 7, 8, 9, 10, 11, 12, 13, 14, 15 or more contiguous amino acids of the amino acid sequence of SEQ ID NO: 1.

An HCV core antigen polypeptide of the present disclosure can be generated using standard peptide synthesis technology or using chemical cleavage methods well known in the art. Alternatively, recombinant methods can be used to generate nucleic acid molecules that encode an HCV core antigen polypeptide. In one embodiment, nucleic acid molecules provide a means to generate defined fragments of an HCV core antigen polypeptide (or variants, homologs or analogs thereof).

In one embodiment, a polynucleotide for expressing an HCV core antigen polypeptide comprises all or part of the nucleic acid sequence of SEQ ID NO: 12. For example, the polynucleotide can be used to express an HCV core antigen polypeptide in a cell-free system, or in a non-human organism or a cell. In some aspects, the organism or cell is a virus, a bacterium (such as E. coli), a yeast cell, a plant cell, an insect cell, or a mammalian cell.

(SEQ ID NO: 12)
atgtccacgaacccaaagccgcagcgtaaaaccaaacgtaacaccaaccg
tcgcccgcaggacgtcaaattccaggtggcggtcagatcgtgggtggcgt
gtacctgagccgcgtcgtggtccgcgcagggtgtacgtgcaacccgtaaa
acctccgagcgttcccagccgcgcggtcgtcgtcagcctatccctaaagc
tcgtcagccggaaggtcgcgcatgggcacaaccgggttacccgtggccac
tgtacggtaacgaaggtatgggctgggcgggttggctgctgagcccacgt
ggttctcgtccgtcttggggtccgactgacccgcgtcgtcgctctcgcaa
tctgggtaaagttatcgacaccctgacctgcggcttcgcggatctgatgg
gctatatcccgctggtaggcgccccgctgggcggcgcagctcgcgctctg
gctcacggcgttcgcgttctggaagatggcgttaactatgcgactggcaa
cctgccgggctgtagcttttccattttcctgaggcgctgagagagcctga
ctattccggcgtccgcg.

In some embodiments, a polynucleotide encoding an HCV core antigen polypeptide shares about 10%, about 15%, about 20%, about 25%, about 30%, about 35%, about 40%, about 45%, about 50%, about 55%, about 60%, about 65%, about 70%, about 75%, about 80%, about 85%, about 90%, about 99%, or 100% similarity with the nucleic acid sequence of SEQ ID NO: 12 or a fragment thereof.

In some embodiments, an HCV core antigen polypeptide can be conveniently expressed in cells (such as E. coli or 293T cells) transfected with a commercially available expression vector. Modifications of an HCV core antigen polypeptide such as covalent modifications are included within the scope of this disclosure. One type of covalent modification includes reacting targeted amino acid residues of an HCV core antigen polypeptide with an organic derivatizing agent that is capable of reacting with selected side chains or the N- or C-terminal residues of the HCV core antigen polypeptide. Another type of covalent modification comprises altering the native glycosylation pattern of the HCV core antigen polypeptide. Another type of covalent modification comprises linking the HCV core antigen polypeptide to one of a variety of nonproteinaceous polymers, e.g., polyethylene glycol (PEG), polypropylene glycol, or polyoxyalkylenes, in the manner set forth in U.S. Pat. Nos. 4,640,835; 4,496,689; 4,301,144; 4,670,417; 4,791,192 or 4,179,337.

In addition to prokaryotes, eukaryotic microbes such as filamentous fungi or yeast are suitable cloning or expression hosts for expressing vectors, including fungi and yeast strains whose glycosylation pathways have been modified to mimic or approximate those in human cells, resulting in the production of a polypeptide or an antibody with a partially or fully human glycosylation pattern. See Gemgross, Nat. Biotech. 22:1409-1414 (2004), and Li et al., Nat. Biotech. 24:210-215 (2006).

Exemplary eukaryotic cells that may be used to express polypeptides include, but are not limited to, COS cells, including COS 7 cells; 293 cells, including 293-6E cells; CHO cells, including CHO-S, DG44. Lec13 CHO cells, and FUT8 CHO cells; PER.C6® cells; and NSO cells. In some embodiments, the antibody heavy chains and/or light chains may be expressed in yeast. See, e.g., U.S. Publication No. US 2006/0270045 A1. In some embodiments, a particular eukaryotic host cell is selected based on its ability to make desired post-translational modifications to the heavy chains and/or light chains. For example, in some embodiments, CHO cells produce polypeptides that have a higher level of sialylation than the same polypeptide produced in 293 cells.

In some embodiments, a polypeptide or antibody disclosed herein is produced in a cell-free system. Exemplary cell-free systems are described, e.g., in Sitaraman et al., Methods Mol. Biol. 498: 229-44 (2009); Spirin, Trends Biotechnol. 22: 538-45 (2004); Endo et al., Biotechnol. Adv. 21: 695-713 (2003).

The HCV core antigen polypeptide of the present disclosure can also be modified to form a chimeric molecule comprising an HCV core antigen polypeptide fused to another, heterologous polypeptide or amino acid sequence. Such a chimeric molecule can be synthesized chemically or recombinantly. In some aspects, an HCV core antigen polypeptide in accordance can comprise a fusion of fragments of the HCV core antigen sequence (amino or nucleic acid) such that a molecule is created that is not, through its length, directly homologous to the amino or nucleic acid sequences shown in SEQ ID NOs: 1-12. Such a chimeric molecule can comprise multiples of the same subsequence of the HCV core antigen polypeptide. A chimeric molecule can comprise a fusion of an HCV core antigen polypeptide with a poly-histidine epitope tag, which provides an epitope to which immobilized nickel can selectively bind, with cytokines or with growth factors. The epitope tag is generally placed at the amino- or carboxyl-terminus of the HCV core antigen polypeptide. In an alternative embodiment, the chimeric molecule can comprise a fusion of an HCV core antigen polypeptide with an immunoglobulin or a particular region of an immunoglobulin. For a bivalent form of the chimeric molecule (also referred to as an “immunoadhesin”), such a fusion could be to the Fc region of an IgG molecule. The Ig fusions preferably include the substitution of a soluble form of an HCV core antigen polypeptide in place of at least one variable region within an Ig molecule. In one embodiment, the immunoglobulin fusion includes the hinge, CH2 and CH3, or the hinge, CH1, CH2 and CH3 regions of an IgG molecule. For the production of immunoglobulin fusions see, e.g., U.S. Pat. No. 5,428,130 issued Jun. 27, 1995.

### III. Camelid HCV Core Antigen Antibodies

In some aspects, provided herein are anti-HCV antibodies, including functional antibody fragments, including those comprising a variable heavy chain. Also provided are molecules containing such antibodies, e.g., fusion proteins and/or recombinant receptors such as chimeric receptors. Among the provided anti-HCV antibodies are antibodies against the HCV core antigen. The antibodies include isolated antibodies.

One aspect of the present disclosure provides antibodies that bind to an HCV core antigen polypeptide. Preferred antibodies specifically bind to an HCV core antigen polypeptide and do not bind (or bind weakly) to peptides or proteins that are not HCV core antigen polypeptides. For example, antibodies that bind to an HCV core antigen polypeptide can bind the HCV core antigen-related proteins such as the homologs or analogs thereof.

HCV core antigen antibodies of the present disclosure are particularly useful in the treatment, diagnosis, diagnostic and prognostic assays, imaging methodologies, and/or prognosis of HCV-related diseases or conditions.

The present disclosure also provides various immunological assays useful for the detection and quantification of HCV core antigen and HCV infection status. Such assays can comprise one or more HCV core antigen antibodies capable of recognizing and binding an HCV core antigen polypeptide, as appropriate. These assays are performed within various immunological assay formats well known in the art, including but not limited to various types of radioimmunoassays, enzyme-linked immunosorbent assays (ELISA), enzyme-linked immunofluorescent assays (ELIFA), and the like.

In other aspects, immunological non-antibody assays of the present disclosure also comprise T cell immunogenicity assays (inhibitory or stimulatory) as well as major histocompatibility complex (MHC) binding assays.

Various methods for the preparation of antibodies are well known in the art. For example, antibodies can be prepared by immunizing a suitable mammalian host using an HCV core antigen polypeptide or fragment, in isolated or immunoconjugated form (Antibodies: A Laboratory Manual, CSH Press, Eds., Harlow, and Lane (1988); Harlow, Antibodies, Cold Spring Harbor Press, NY (1989)). In addition, fusion proteins of an HCV core antigen polypeptide can also be used, such as an HCV core antigen GST-fusion protein. In a particular embodiment, a GST fusion protein comprising all or most of the amino acid sequence of SEQ ID NOs: 1-11 is produced, then used as an immunogen to generate appropriate antibodies. In another embodiment, an HCV core antigen polypeptide is synthesized and used as an immunogen.

In addition, naked DNA immunization techniques known in the art are used to generate an immune response to the encoded immunogen (for review, see Donnelly et al., 1997, Ann. Rev. Immunol. 15: 617-648). For example, all or a part of SEQ ID NO: 12 can be used to generate an immune response to the encoded immunogen, i.e., an HCV core antigen polypeptide.

The amino acid sequence of an HCV core antigen polypeptide, such as one shown in SEQ ID NOs: 1-11 can be analyzed to select specific regions of the HCV core antigen polypeptide for generating antibodies. For example, hydrophobicity and hydrophilicity analyses of the HCV core antigen amino acid sequence are used to identify hydrophilic regions in the HCV core antigen structure. Regions of the HCV core antigen that show immunogenic structure, as well as other regions and domains, can readily be identified using various other methods known in the art, such as Chou-Fasman, Hopp and Woods, Kyte-Doolittle, Janin, Bhaskaran and Ponnuswamy, Deleage and Roux, Garnier-Robson, Eisenberg, Karplus-Schultz, or Jameson-Wolf analysis. Thus, each region identified by any of these programs or methods is within the scope of the present disclosure. Methods for the generation of HCV core antigen antibodies are further illustrated by way of the examples provided herein. Methods for preparing a protein or polypeptide for use as an immunogen are well known in the art. Also well known in the art are methods for preparing immunogenic conjugates of a protein with a carrier, such as BSA, KLH or other carrier protein. In some circumstances, direct conjugation using, for example, carbodiimide reagents are used; in other instances linking reagents such as those supplied by Pierce Chemical Co., Rockford, Ill., are effective. Administration of an HCV core antigen immunogen is often conducted by injection over a suitable time period and with use of a suitable adjuvant, as is understood in the art. During the immunization schedule, titers of antibodies can be taken to determine adequacy of antibody formation.

HCV core antigen monoclonal antibodies can be produced by various means well known in the art. For example, immortalized cell lines that secrete a desired monoclonal antibody are prepared using the standard hybridoma technology of Kohler and Milstein or modifications that immortalize antibody-producing B cells, as is generally known. Immortalized cell lines that secrete the desired antibodies are screened by immunoassay in which the antigen is an HCV core antigen polypeptide. When the appropriate immortalized cell culture is identified, the cells can be expanded and antibodies produced either from in vitro cultures or from ascites fluid.

Reactivity of an HCV core antigen antibody with an HCV core antigen polypeptide can be established by a number of well known means, including Western blot, immunoprecipitation, ELISA, and FACS analyses using, as appropriate, an HCV core antigen polypeptide, an HCV core antigen expressing cells or extracts thereof. An HCV core antigen antibody or fragment thereof can be labeled with a detectable marker or conjugated to a second molecule. Suitable detectable markers include, but are not limited to, a radioisotope, a fluorescent compound, a bioluminescent compound, chemiluminescent compound, a metal chelator or an enzyme. Further, bi-specific antibodies specific for two or more HCV core antigen epitopes are generated using methods generally known in the art. Homodimeric antibodies can also be generated by cross-linking techniques known in the art (e.g., Wolff et al., Cancer Res. 53: 2560-2565).

In one aspect, because single domain VHH antibodies from camelids are well suited for large scale production of antibodies, single domain VHH antibodies specific for HCV core antigen are provided in the present disclosure. In some embodiments, the present disclosure also includes single-chain antibody fragments, typically comprising linker(s) joining two antibody domains or regions, such two or more single domain VHH antibodies (which can be the same or different). The linker typically is a peptide linker, e.g., a flexible and/or soluble peptide linker, such as one rich in glycine and serine. In some aspects, the linkers rich in glycine and serine (and/or threonine) include at least 80, 85, 90, 91, 92, 93, 94, 95, 96, 97, 98, or 99% such amino acid(s). In some embodiments, they include at least at or about 50%, 55%, 60%, 70%, or 75%, glycine, serine, and/or threonine. In some embodiments, the linker is comprised substantially entirely of glycine, serine, and/or threonine. The linkers generally are between about 5 and about 50 amino acids in length, typically between at or about 10 and at or about 30, e.g., 10, 11, 12, 13, 14, 15, 16, 17, 18, 19, 20, 21, 22, 23, 24, 25, 26, 27, 28, 29, or 30, and in some examples between 10 and 25 amino acids in length. Exemplary linkers include linkers having various numbers of repeats of the sequence GGGGS (4GS) or GGGS (3GS), such as between 2, 3, 4, and 5 repeats of such a sequence.

Currently mice are the most widely used host for generating monoclonal antibodies, but antibody yields are generally low. Rabbits usually generate better immune response than mice for many immunogens. However, technologies to generate monoclonal rabbit antibodies are not as widely available due to limited availability of fusion partners for hybridomas.

Camelids make a type of antibodies with a homodimeric heavy-chain that is devoid of light chains. The antigen-binding sites of these antibodies are located on a single variable domain (VHH), which also has three hypervariable regions as well as increased variability on the framework regions. See Muyldermans et al., Recognition of antigens by single-domain antibody fragments: the superfluous luxury of paired domains, Trends Biochem Sci, 2001, 26(4):230-35. VHHs often have longer CDR1 and CDR3 regions to increase the structural repertoire of the antigen-binding site and compensate for the absence of the VL CDRs. This special structural feature also allows the paratope to be more concentrated over a smaller area so that small hidden epitopes can still be targeted by VHH.

Nonetheless, VHH antibodies tend to target different epitopes from those of regular antibodies. Particularly, camelids are able to produce high affinity VHH antibodies for haptens and peptides which are otherwise difficult to generate from mice or rabbits through conventional antibody production techniques.

Antigen-specific VHHs can be selected using a number of genetic engineering techniques from synthetic or naive VHH libraries. See Olichon et al., Preparation of a naive library of camelid single domain antibodies, Methods Mol Biol, 2012, 911:65-78. However, these often results in antibodies with lower affinity for small molecules. See Alvarez-Rueda et al., Generation of llama single-domain antibodies against methotrexate, a prototypical hapten, Mol Immunol, 2007, 44(7):1680-90. In addition, stability and yield are often a problem associated with antibodies developed from synthetic libraries. On the other hand, immunizing llamas by repeated subcutaneous injections reliably gives affinity-matured antibodies as in any other animal system (e.g., goat or rabbit).

The size of the library is often a limiting factor for the throughput and efficiency of library screening, especially when large numbers of antibodies need to be generated. In the case of screening a VHH library, it usually involves cloning the VHH repertoire from B lymphocytes into a phage display vector. After several rounds of panning, individual clones with antigen-specific VHH can be identified. This method is more efficient than corresponding techniques to identify antigen binding partners for conventional antibodies in scFv or Fab format, where VH and VL genes are separately cloned and recombined. For example, from 10⁵ B cells, 10⁵ different VHH genes need to be amplified. If however, a library for both VH and VL regions is created, 10⁵ VH genes will need to be joined to 10⁵ different VL genes in 10¹⁰ clones to cover the entire repertoire. See Campaign, Diagnosis and treatment of Hepatitis C: A technical landscape, Oct. 1, 2013, available at msfaccess.org/sites/default/files/MSF_assets/HepC/Docs/HepC_Report DxTxTech_ENG_2013_FINAL.pdf.

In a related aspect, the present disclosure provides a method of producing a library of expression vectors encoding VH (and/or VL) domains of camelid antibodies, said method comprising the steps: a) amplifying regions of nucleic acid molecules encoding VH (and/or VL) domains of camelid antibodies to obtain amplified gene segments, each gene segment containing a sequence of nucleotides encoding a VH domain or a sequence of nucleotides encoding a VL domain of a camelid antibody, and b) cloning the gene segments obtained in a) into expression vectors, such that each expression vector contains at least a gene segment encoding a VH domain and/or a gene segment encoding a VL domain, whereby a library of expression vectors is obtained.

In one embodiment, the nucleic acid amplified in step a) comprises cDNA or genomic DNA prepared from lymphoid tissue of a camelid, said lymphoid tissue comprising one or more B cells, lymph nodes, spleen cells, bone marrow cells, or a combination thereof. In one aspect, peripheral blood lymphocytes (PBLs) or PBMCs can be used as a source of nucleic acid encoding VH and VL domains of camelid antibodies, i.e. there is sufficient quantity of plasma cells (expressing antibodies) present in a sample of PBMCs to enable direct amplification. This is advantageous because PBMCs can be prepared from a whole blood sample taken from the animal (camelid). This avoids the need to use invasive procedures to obtain tissue biopsies (e.g. from spleen or lymph node), and means that the sampling procedure can be repeated as often as necessary, with minimal impact on the animal. For example, it is possible to actively immunize the camelid, remove a first blood sample from the animal and prepare PBMCs, then immunize the same animal a second time, either with a “boosting” dose of the same antigen or with a different antigen, then remove a second blood sample and prepare PBMCs.

Accordingly, a particular embodiment of this method of the present disclosure may involve: preparing a sample containing PBMCs from a camelid, preparing cDNA or genomic DNA from the PBMCs and using this cDNA or genomic DNA as a template for amplification of gene segments encoding VH or VL domains of camelid antibodies.

In one embodiment the lymphoid tissue (e.g. circulating B cells) is obtained from a camelid which has been actively immunized, as described elsewhere herein. However, this embodiment is non-limiting and it is also contemplated to prepare non-immune libraries and libraries derived from lymphoid tissue of diseased camelids, also described elsewhere herein.

Conveniently, total RNA (or mRNA) can be prepared from the lymphoid tissue sample (e.g. peripheral blood cells or tissue biopsy) and converted to cDNA by standard techniques. It is also possible to use genomic DNA as a starting material.

This aspect of the present disclosure encompasses both a diverse library approach, and a B cell selection approach for construction of the library. In a diverse library approach, repertoires of VH and VL-encoding gene segments may be amplified from nucleic acid prepared from lymphoid tissue without any prior selection of B cells. In a B cell selection approach, B cells displaying antibodies with desired antigen-binding characteristics may be selected, prior to nucleic acid extraction and amplification of VH and VL-encoding gene segments.

Various conventional methods may be used to select camelid B cells expressing antibodies with desired antigen-binding characteristics. For example, B cells can be stained for cell surface display of conventional IgG with fluorescently labelled monoclonal antibody (mAb, specifically recognizing conventional antibodies from llama or other camelids) and with target antigen labelled with another fluorescent dye. Individual double positive B cells may then be isolated by FACS, and total RNA (or genomic DNA) extracted from individual cells. Alternatively cells can be subjected to in vitro proliferation and culture supernatants with secreted IgG can be screened, and total RNA (or genomic DNA) extracted from positive cells. In a still further approach, individual B cells may be transformed with specific genes or fused with tumor cell lines to generate cell lines, which can be grown “at will”, and total RNA (or genomic DNA) subsequently prepared from these cells.

Instead of sorting by FACS, target specific B cells expressing conventional IgG can be “panned” on immobilized monoclonal antibodies (directed against camelid antibodies) and subsequently on immobilized target antigen. RNA (or genomic DNA) can be extracted from pools of antigen specific B cells or these pools can be transformed and individual cells cloned out by limited dilution or FACS.

B cell selection methods may involve positive selection, or negative selection.

Whether using a diverse library approach without any B cell selection, or a B cell selection approach, nucleic acid (cDNA or genomic DNA) prepared from the lymphoid tissue is subject to an amplification step in order to amplify gene segments encoding individual VH domains or VL domains.

Total RNA extracted from the lymphoid tissue (e.g. peripheral B cells or tissue biopsy) may be converted into random primed cDNA or oligo dT primer can be used for cDNA synthesis, alternatively Ig specific oligonucleotide primers can be applied for cDNA synthesis, or mRNA (i.e. poly A RNA) can be purified from total RNA with oligo dT cellulose prior to cDNA synthesis. Genomic DNA isolated from B cells can be used for PCR.

In some aspects, provided herein are methods of producing renewable antibodies against HCV core antigen from camelids, specifically llamas. Camelids produce single-domain heavy-chain antibodies (VHH) in addition to conventional antibodies. See Hamers-Casterman et al., Naturally occurring antibodies devoid of light chains, Nature, 1993, 363(6428):446-8. The antigen specific VHHs are the smallest binding units produced by the immune systems. Compared to conventional antibodies, in some aspects, camelid VHHs have advantages which make them a better system for generating renewable antibodies on a large scale.

In some embodiments, the camelid is first immunized with an HCV core antigen polypeptide of the present disclosure. The HCV core antigen polypeptide can be the full length HCV core antigen or a fragment thereof, and can be a fusion protein with one or more tags. In some embodiments, the same animal is immunized a second time (or additional times), either with a “boosting” dose of the same HCV core antigen polypeptide or with a different HCV core antigen polypeptide. For example, the camelid can be initially immunized with an HCV core antigen fragment fused to a tag, and then boosted with a full length HCV core antigen and/or an HCV core antigen fragment without the tag, or vice versa.

First, Camelid single-domain antibody fragments make the VHHs more suited for construction of large libraries for in vitro display selection systems. See Arbabi Ghahroudi et al., Selection and identification of single domain antibody fragments from camel heavy-chain antibodies, FEBS Lett, 1997, 414(3):521-6. VHH libraries generated from immunized camelids retain full functional diversity, whereas the conventional antibody libraries suffer from diminished diversity due to reshuffling of VL and VH domains during library construction. See Harmsen et al., Properties, production, and applications of camelid single-domain antibody fragments, Appl Microbiol Biotechnol, 2007, 77(1):13-22; Harmsen et al., Llama heavy-chain V regions consist of at least four distinct subfamilies revealing novel sequence features, Mol Immunol, 2000, 37(10):579-90; van der Linden et al., Induction of immune responses and molecular cloning of the heavy chain antibody repertoire of Lama glama, J Immunol Methods, 2000, 240(1-2): 185-95; Frenken et al., Isolation of antigen specific llama VHH antibody fragments and their high level secretion by Saccharomyces cerevisiae, J Biotechnol, 2000, 78(1):11-21. In vitro selection systems immediately provide the identity of genes and corresponding sequences of antibodies selected against a particular target. By introducing additional mutations and constructing secondary libraries, antibody affinity and specificity can be further tailored. Usability of these antibodies can be further expanded through modifications by simple subcloning to create fusion products to enzymes, tags, fluorescent proteins or Fc domains. In some aspects, provided herein are fusion VHH antibodies with rabbit Fc, and the functionality of the fusion antibodies in LFIA devices is demonstrated. In some aspects, the uniform Fc domain on antibodies also makes them easier to be applied in multiplexed immunoassays.

Second, by adopting different binding patterns, VHHs can specifically interact with small molecules. See Fanning et al., An anti-hapten camelid antibody reveals a cryptic binding site with significant energetic contributions from a nonhypervariable loop, Protein Sci, 2011, 20(7):1196-207. Small molecules such as herbicides, caffeine, mycotoxins, trinitrotoluene, steroids, and therapeutic drugs have all been successfully used as haptens to generate specific VHHs from both naïve and immunized camelid VHH display libraries. See Yau et al., Selection of hapten-specific single-domain antibodies from a non-immunized llama ribosome display library, J Immunol Methods, 2003, 281(1-2):161-75; Sheedy et al., Selection, characterization, and CDR shuffling of naive llama single-domain antibodies selected against auxin and their cross-reactivity with auxinic herbicides from four chemical families, J Agric Food Chem, 2006, 54(10):3668-78; Ladenson et al., Isolation and characterization of a thermally stable recombinant anti-caffeine heavy-chain antibody fragment, Anal Chem, 2006, 78(13):4501-8; Alvarez-Rueda et al., Generation of llama single-domain antibodies against methotrexate, a prototypical hapten, Mol Immunol, 2007, 44(7):1680-90; Doyle et al., Cloning, expression, and characterization of a single-domain antibody fragment with affinity for 15-acetyl-deoxynivalenol, Mol Immunol, 2008, 45(14):3703-13; Anderson et al., TNT detection using llama antibodies and a two-step competitive fluid array immunoassay, J Immunol Methods, 2008, 339(1):47-54; and Kobayashi et al., “Cleavable” hapten-biotin conjugates: preparation and use for the generation of anti-steroid single-domain antibody fragments, Anal Biochem, 2009, 387(2):257-66. Anti-peptide VHHs have also been successfully generated from immunized camels. See Aliprandi et al., The availability of a recombinant anti-SNAP antibody in VHH format amplifies the application flexibility of SNAP-tagged proteins, J Biomed Biotechnol, 2010, 2010:658954. Therefore, both synthetic peptides and purified proteins may be used as immunogen to guide the immune response to specific epitopes.

Third, single-domain antibody fragments are well expressed in microorganisms and have a high apparent stability and solubility. In some aspects, without much optimization, several milligrams of VHHs can be purified from each liter of bacterial culture. These properties greatly facilitate the production of such antibodies at larger number/quantity at significant lower cost, therefore will further reduce the cost of immunoassays. This is more important to resource-poor areas where usually also have higher incidences of various HBV and HCV.

In some embodiments, single domain antibodies and their binding to cognate antigens are extremely stable and resistant to high concentrations of denaturant. This property makes it possible to perform specific immunoassays under denaturing conditions. In the case of many viral infections, including CMV, HCV, HIV, etc., patients produce self-defense antibodies which bind on the viral antigen. In order to detect these antigens, the antibody-antigen complexes have to be destroyed and the host antibodies need to be denatured. The denaturant will then need to be removed from the assay prior to adding the detection antibody to protect the detection antibody from being damaged. All these procedures make the current immunoassays for viral antigen detection complicated. As a result, no rapid tests have been successfully developed for HCV. On the other hand, VHH single domain antibodies can be applied in lateral flow immunoassays for rapid detection of antigen in the presence of strong denaturant. Typically, removal of the denaturant from the assay is not necessary with these antibodies. Therefore, in some aspects, these antibodies are used to detect viral antigens directly from body fluid under denaturing conditions, for example, to provide rapid tests for point-of-care (POC) detection of these viral antigens.

In one aspect, provided herein is an immunization and in vitro screening platform that is well suited to generate large numbers of high affinity VHH antibodies. In one aspect, provided herein is an immunization and in vitro screening platform for generating high affinity antibodies to HCV core antigen.

In some aspects, the provided antibodies have one or more specified functional features, such as binding properties, including binding to particular epitopes, such as epitopes that are similar to or overlap with those of other antibodies, the ability to compete for binding with other antibodies, and/or particular binding affinities.

In some embodiments, such properties are described in relation to properties observed for another antibody, e.g., a reference antibody or a host antibody (for example, host antibodies in seroconversion). For example, in some embodiments, the antibody specifically binds to an epitope that overlaps with the epitope of HCV core antigen bound by a host antibody, such as antibodies that bind to the same or a similar epitope as the host antibody. In some embodiments, the antibody competes for binding to HCV core antigen with the host antibody. An antibody “competes for binding” to HCV core antigen with a reference or host antibody if it competitively inhibits binding of the reference or host antibody to HCV core antigen, and/or if the reference or host antibody competitively inhibits binding of the antibody to HCV core antigen. An antibody competitively inhibits binding of a reference or host antibody to an antigen if the presence of the antibody in excess detectably inhibits (blocks) binding of the other antibody to its antigen. A particular degree of inhibition may be specified. In some embodiments, addition of the provided antibody in excess, e.g., 1-, 2-, 5-, 10-, 50- or 100-fold excess, as compared to the amount or concentration of the reference or host antibody, inhibits binding to the antigen by the reference or host antibody (or vice versa). In some embodiments, the inhibition of binding is by at least 50%, and in some embodiments by at least 75%, 90% or 99%. In some aspects, the competitive inhibition is as measured in a competitive binding assay (see, e.g., Junghans et al., Cancer Res. 1990:50:1495-1502). Competitive inhibition assays are known and include ELISA-based, flow cytometry-based assays, and RIA-based assays. In some aspects, competitive inhibition assays are carried out by incorporating an excess of an unlabeled form of one of the antibodies and assessing its ability to block binding of the other antibody, which is labeled with a detectable marker, such that degree of binding and reduction thereof can be assessed by detection of the label or marker.

HCV is highly heterogeneous and has been classified into different genotypes based on at least 67% similarity of the nucleotide sequences. HCV genotypes display significant differences in their global distribution and prevalence. The genotype of the HCV strain also appears to be an important determinant of the severity and aggressiveness of liver infection, as well as patient response to anti-viral therapy (interferon and ribavirin). See Turhan et al., Investigation of the genotype distribution of hepatitis C virus among Turkish population in Turkey and various European countries, Chin Med J (Engl), 2005, 118(16):1392-94. Genotype 1 is the most common HCV genotype in the United States. Yet patients with genotypes 2 and 3 are almost three times more likely than patients with genotype 1 to respond to therapy with alpha interferon or the combination of alpha interferon and ribavirin. See Qiu et al., HCV genotyping using statistical classification approach, J Biomed Sci, 2009, 16:62.

The monoclonal antibody in the immunoassay for HCV core antigen available from Abbott used a recombinant c11 antigen (residues 1-160 of genotype 2a) as immunogen. See Ergunay et al., Utility of a commercial quantitative hepatitis C virus core antigen assay in a diagnostic laboratory setting, Diagn Microbiol Infect Dis, 2011, 70(4):486-91. There are also multiple antibodies available for research applications (Abcam), mostly generated with recombinant polypeptides covering partial sequences of the core antigen. A sequence alignment of the first 180 amino acids for genotype 1a, 1b, 2a and 2b shows that the sequences are almost 90% identical. Since the genotype 1 is the most dominant form that constitutes up to 70% of the total incidence, the core antigen from genotype 1 is used in one embodiment as immunogen for generating VHH antibodies. Recombinant protein of genotype 1b is commercially available (e.g., from Prospec, Cat # HCV-241). Genotype 1a and 1b have only one amino acid difference in the aligned region (underlined and bolded in the sequence alignment below).

| | |
|---|---|
| 2a (SEQ ID NO: 13) | MSTNPKPQRKTKRNTNRRPQDVKFPGGGQIVGGVYLLPRRGPRLGVRAARKTSERSQPRG |
| 2b (SEQ ID NO: 14) | MSTNPKPQRKTKRNTNRRPQDVKFPGGGQIVGGVYLLPRRGPRLGVRAPRKTSERSQPRG |
| 1b (SEQ ID NO: 15) | MSTNPKPQRKTKRNTNRRPQDVKFPGGGQIVGGVYLLPRRGPRLGVRATRKTSERSQPRG |
| 1a (SEQ ID NO: 16) | MSTNPKPQRKTKRNTNRRPQDVKFPGGGQIVGGVYLLPRRGPRLGVRATRKTSERSQPRG |
| consensus | ************************************************.*********** |
| 2a (SEQ ID NO: 13) | RRQPIPKDRRSTGKSWGKPGYPWPLYGNEGLGWAGWLLSPRGSRPSWGPTDPRHRSRNVG |
| 2b (SEQ ID NO: 14) | RRQPIPKDRRSTGKSWGKPGYPWPLYGNEGCGWAGWLLSPRGSRPTWGPTDPRHRSRNLG |
| 1b (SEQ ID NO: 15) | RRQPIPKARRPEGRAWAQPGYPWPLYGNEGLGWAGWLLSPRGSRPSWGPTDPRRRSRNLG |
| 1a (SEQ ID NO: 16) | RRQPIPKARRPEGRAWAQPGYPWPLYGNEGCGWAGWLLSPRGSRPSWGPTDPRRRSRNLG |
| consensus | ******* **. *::*.:************ **************:*******:****:* |
| 2a (SEQ ID NO: 13) | KVIDTLTCGFADLMGYIPVVGAPLGGVARALAHGVRVLEDGVNFATGNLPGCSFSIFLLA |
| 2b (SEQ ID NO: 14) | RVIDTITCGFADLMGYIPVVGAPVGGVARALAHGVRVLEDGVNYATGNLPGCSFSIFLLA |
| 1b (SEQ ID NO: 15) | KVIDTLTCGFADLMGYIPLVGAPLGGAARALAHGVRVLEDGVNYATGNLPGCPFSVFLLA |
| 1a (SEQ ID NO: 16) | KVIDTLTCGFADLMGYIPLVGAPLGGAARALAHGVRVLEDGVNYATGNLPGCSFSIFLLA |
| consensus | :****:************:****:**.****************:********.**:**** |

In one embodiment, a llama (male or female) is immunized following an optimized immunization and boost schedule. In one aspect, specific anti-sera titer is determined at 40 days, 60 days, 80 days and 100 days post immunization. In one aspect, three proteins are used for coating the ELISA plates: 1) the immunogen; 2) the recombinant core protein that covers amino acid 1-120 (e.g., from Creative Biolabs); and 3) Bf-galactosidase (the fusion partner in the recombinant protein). Typically, 96-well plates are coated with antigen as indicated. Following blocking and washing, 1:10 serial diluted anti-sera are added to each well. Dilutions in the range of 1:10,000 to 1:10,000,000 are adequate in most cases. Bound antibodies are detected with HRP-conjugated goat anti-llama antibody. In one aspect, the ELISA tests are carried out with or without 3-galactosidase (e.g., from USbio, Cat# G1041-05) as blocker in the binder buffer. In one aspect, positive high titers in both coated plates and reactions are not blocked by β-galactosidase indicate the presence of HCV core antigen specific antibodies in the serum. In some embodiments, positive reactions are seen at 60 days and the titer continues to rise afterwards. Production bleed are typically collected on day 80 and 100 when the titer reaches the highest.

In cases where no specific titer is detected on day 60 due to low immune response, a different llama can be immunized. Recombinant antigen of a different source can be used, such as a recombinant protein (1-120 amino acid with His tag) purified from pichia (e.g., from Creative Biolabs). In other embodiments, synthetic peptides are used for immunization (after conjugation to KLH) to cover different regions of the core antigen.

In one aspect, provided herein is a method for single domain antibody library construction. Peripheral blood mononuclear cells (PBMC) are isolated by Ficoll gradient and total RNA is isolated from these cells. Each production bleed typically results in recovery of ˜5×10⁸ cells from 500 ml of blood. The cell number and integrity are examined under microscope with Trypan Blue staining. PBMC cells are then processed, and VHH libraries are constructed by RT-PCR. Based on bioinformatics analysis and sequencing of VHH clones, sets of primers are designed for reverse transcription and PCR. A phage display vector with His-tag is then used for cloning the VHH library. Typically, >10⁹ independent clones for each library are obtained. One library is constructed for each immunized llama.

In one aspect, provided herein is a method for VHH library screening. Specific high affinity binders are selected according to an optimized in vitro screening protocol. To isolate highly specific antibody clones, two approaches are incorporated in the protocol. First, core-antigen coated plates and biotin-core-antigen/streptavidin magnetic beads are used alternatively in subsequent screening steps to prevent the isolation of phage that binds to the plate or magnetic beads non-specifically. For example, biotin-core-antigen/streptavidin magnetic beads are used for the first round of screening for higher handling volume, since the starting number of phages is the largest during the first round in order to cover the entire library. For the second round of screening, core-antigen coated plates are used to select phage. Those phages that non-specifically bind on the magnetic beads and are isolated from the first round of screening have a less chance of binding on the plate. This way, the background is dramatically reduced. Second, β-galactosidase (the fusion partner in the recombinant protein) is used in the hybridization buffer to block the binding of galactosidase specific antibodies to the plate or beads. The binding conditions for each round of panning/screening can be adjusted to obtain desired clones, including input antigen concentration, input number of phage, detergent concentration, and number of washing steps. Typically, between about 10% and about 50% of clones are positive high affinity binders after three rounds of screening.

To further increase the chance of isolating pairing antibodies for sandwich immunoassays and to improve the efficiency, a recombinant protein (1-120 amino acid with His tag) purified from pichia (e.g., from Creative Biolabs) can be used to screen the antibody library. When screening the library with the immunogen (2-192 amino acids), the shorter peptide (1-120 amino acids) can be used as a blocker to favor the isolation of antibodies against epitopes outside of the 1-120 amino acids. Antibodies isolated with two different antigens have a better chance to form pair in sandwich immunoassays.

In one aspect, provided herein is a method for high affinity VHH clone isolation. The phage display system disclosed herein has several convenient features. First, by changing culture conditions, the system can be induced to either preferentially display antibodies on phage particles for screening of phage, or secreting soluble antibodies into the culture media for direct ELISA to identify positive clones. By switching host cells, the system can produce soluble VHH proteins for pilot scale purification and characterization without further subcloning. The VHH sequences are flanked by two rare restriction sites that are also built into our expression vector for Fc fusion protein expression. Once positive clones are identified, the VHH sequence can be easily subcloned into an Fc fusion protein expression vector to produce VHH-Fc proteins. Individual clones producing high affinity specific antibodies will be identified by ELISA. The gene sequence of each clone will be analyzed and aligned to each other and other VHH sequences to identify the framework regions and CDRs of each antibody. Typically, high affinity specific binders are amplified through multiple rounds of screening and therefore multiple clones show identical sequences for the same epitope. Based on sequence information, clones with different sequences can be sorted into different groups. Those clones with more significant differences in the CDR can have a different binding epitope on the antigen. One or two amino acid differences in CDR1 or CDR3 can cause variation in the affinity on the same epitope. Antibodies with multiple amino acid differences spanning CDR1 to CDR3 usually bind on different epitopes. These sequence information therefore can be used for selecting pairing antibodies in the next steps.

In one aspect, the affinity and specificity of the VHH antibodies are examined. In one aspect, the antibody is expressed in rabbit Fc fusion format for lateral flow assays. In another aspect, pairing antibodies for sandwich immunoassays are identified. VHH antibody proteins are purified from the positive clones from E. coli culture. Several milligrams of pure VHH protein are usually obtained from each liter of culture. Purity of protein is examined on SDS-PAGE followed by Coomassie blue staining of the gel. Protein concentration is determined with Bradford assay using Bovine Gamma Globulin Standard (e.g., from Pierce, Cat#23212).

In one aspect, polyclonal antibody raised in goat against llama IgG is used in detecting VHH antibodies in ELISA to determine affinities of the antibodies to their cognate antigens. ELISA plates are coated with BSA-peptide conjugates at 1 ng/μl. Serial diluted purified antibodies can be added to antigen coated wells. After washing steps, VHH antibody binding to HCV core antigen can be detected by HRP-conjugated goat anti-llama antibody. TMB substrate can be used to develop color signal of the ELISA. The apparent kD for each purified VHH antibody can be obtained by non-linear regression curve fitting. Typically, the goat anti-llama antibody has a kD of about 10 nM to VHHs (measured by ELISA). Although the affinity of the secondary antibody to VHH sets the limit on measurable kD of VHHs to their cognate antigens, this method typically provides a quick ranking of isolated VHH clones without much manipulation.

Specificity of each VHH can be determined by two ELISA methods. VHH antibodies can be used directly in ELISA to detect binding to the HCV core antigen and β-galactosidase (as described above). Those VHHs that bind to the core antigen, but not the β-galactosidase can be further tested in competition ELISA. 96-well plates can be coated with HCV core antigen at 0.1 ng/μl, HCV core antigen and β-galactosidase can be serial diluted with binding buffer containing VHH (concentration determined by kD analysis) and added to each well. In cases where the antibody is specific to the core antigen, the core antigen competes with the coated protein for binding of the VHH; the β-galactosidase does not compete for the binding of antibody. A competition/inhibition curve can be constructed to determine the specificity.

Those antibodies perform well in ELISA under both conditions can be selected for further development.

In another aspect, provided herein is a method for production of VHH fusion antibodies, such as VHH-rFc fusion antibodies. In one aspect, rabbit Fc fusion VHHs are produced. Due to the effect of dimerization, the antibody affinity and specificity are usually improved by fusion to Fc fragments. See Aliprandi et al., The availability of a recombinant anti-SNAP antibody in VHH format amplifies the application flexibility of SNAP-tagged proteins, J Biomed Biotechnol, 2010, 2010:658954.

In some embodiments, an E. coli expression system is used to express antibodies, including single domain, Fab, or full length IgG. The system uses a periplasmic secretion signal to direct expressed protein into the reducing environment of periplasm to facilitate disulfide bond formation and keep the antibodies soluble. In some embodiments, multiple VHH-rFc proteins at ˜mg/L scale are produced in shaker flasks. These antibodies are used to conjugate colloidal gold and applied in lateral flow immunoassays (see the Examples). In one aspect, the bacterial expression system provides a renewable and low cost source for unlimited antibodies, therefore is a better choice for applications in rapid tests.

Genes of those VHH clones that give highest affinities and specificities are subcloned into the expression vector with built-in rabbit Fc region containing the hinge, CH2 and CH3 domains. In one aspect, the vectors are designed with compatible restriction sites for single step ligation and subcloning. The resulted fusion proteins (rFc-VHH) can be easily expressed and purified with protein A/G affinity chromatography at large quantities and high purity. Typically, ˜10 mg of each antibody is purified for rapid test devices. Affinity of the fusion antibodies to their antigens can be re-determined using HRP conjugated goat anti-rabbit polyclonal antibodies, which usually is not a limiting factor in affinity measurements using ELISAs.

Specificity of the antibodies is examined with Western-blot following SDS-PAGE of patient serum containing the core antigen (e.g., from Meridian Life Sciences). The specificity and affinity of selected antibodies can be further determined by label-free, real time kinetic assays (e.g., Octet, Forte Bio). Unlike rough estimates of kinetic information from IC50 values obtained via ELISAs, real-time kinetic measurements offer a direct and more realistic depiction of molecular interactions. Kinetic constants such as ka, kd, K_{D} can be determined. Selected antibodies can be analyzed for their specificity and affinity with the Octet instrument and methods.

In the event that the affinity or specificity of the antibodies is not satisfactory, an affinity maturation steps can be carried out to further improve the antibodies. First, screening is done at lower stringencies to select several candidate clones. Based on the sequence of these candidate clones, antibody affinity/specificity maturation can be performed. DNA sequences at selected positions in the complementarity determination region (CDR), usually CDR3 can be randomized or changed in length to create a sub-library. This library can be subjected to screening as described above to identify specific binders. Typically, the affinity maturation procedures yield antibodies with ˜10 to 1000 fold improved affinities.

In one aspect, provided herein is a method for finding pairing antibodies for sandwich ELISA. Typically, pairing antibodies with different binding epitopes on the antigen are used for sandwich ELISA. Sandwich ELISA can be performed using matrix of VHH antibodies. Capture VHH antibodies can be coated on the plate. After blocking and washing, HCV core antigen can be added to the plate and can be captured by the VHH antibody. Rabbit Fc fusion VHH can be used as detection antibody, which is further detected with HRP-goat anti-rabbit Fc antibody. The Sandwich ELISA can also be performed in the reverse order: coating VHH-rFc on the plate, and detecting with VHH antibody which is His-tagged, which can be detected with mouse anti-His Tag antibody. With differential/subtractive screening using two different antigens, pairs of antibodies for sandwich ELISA can be identified.

All candidate antibodies can be tested with recombinant HCV core antigens of different genotype, available from Meridian Life Sciences (available at meridianlifescience.com/products/results_2.aspx?searchbox=HCV&page=1&group=0). Their sensitivity and specificity can be compared to those of antibodies that are commercially available (e.g., from Abcam and Meridian Life Sciences).

In another aspect, provided herein is a method for determining sensitivity and detection limit of ELISA with seroconversion panels. Selected antibodies can be used to test HCV serum samples. HCV seroconversion panels are commercially available from multiple sources, for example Zeptometrix (available at zeptometrix.com/store/quality-control-panels/seroconversion/hcv/hcv-seroconversion-panel-donor-490105274/). These samples are well documented with test results related to HCV using several commercially available methods, including EIA and RNA PCR.

In one embodiment, two seroconversion panels (total of 10 to 20 samples) are tested in ELISA with one pair of HCV core antigen antibodies. Prior to the assay, each serum is fully denatured to dissociate antibody-bound core antigen, lyse viral particles and expose core antigen, and inactivate human antibody. Each sample is then serial diluted to 6 concentrations and tested with the pair of selected antibodies. Sensitivity and detection limit of the assay are determined. The results are compared to those from other methods provided by the supplier. These antibodies are then used to further develop diagnostic ELISA kits and rapid test LFIA devices.

### IV. Methods for Detection and Diagnosis

Also provided herein are methods involving use of the provided binding molecules, e.g., antibodies, in detection of HCV core antigen, for example, in diagnostic and/or prognostic methods in association with HCV infection. The methods in some embodiments include incubating a biological sample with the antibody and/or administering the antibody to a subject. In certain embodiments, the contacting is under conditions permissive for binding of the anti-HCV core antigen antibody, such as a single domain VHH antibody, to HCV core antigen, and detecting whether a complex is formed between the anti-HCV core antigen antibody and HCV core antigen. Such a method may be an in vitro or in vivo method.

In some embodiments, a sample, such as a cell, tissue sample, lysate, composition, or other sample derived therefrom is contacted with the anti-HCV core antigen antibody and binding or formation of a complex between the antibody and the sample (e.g., HCV core antigen in the sample) is determined or detected. When binding in the test sample is demonstrated or detected as compared to a reference cell of the same tissue type, it may indicate the presence of an associated disease or condition. In some embodiments, the sample is from human tissues.

Various methods known in the art for detecting specific antibody-antigen binding can be used. Exemplary immunoassays include fluorescence polarization immunoassay (FPIA), fluorescence immunoassay (FIA), enzyme immunoassay (EIA), nephelometric inhibition immunoassay (NIA), enzyme linked immunosorbent assay (ELISA), and radioimmunoassay (RIA). An indicator moiety, or label group, can be attached to the subject antibodies and is selected so as to meet the needs of various uses of the method which are often dictated by the availability of assay equipment and compatible immunoassay procedures. Exemplary labels include radionuclides (e.g. ¹²⁵I, ¹³¹, ³⁵S, ³H, or ³²P), enzymes (e.g., alkaline phosphatase, horseradish peroxidase, luciferase, or β-glactosidase), fluorescent moieties or proteins (e.g., fluorescein, rhodamine, phycoerythrin, GFP, or BFP), or luminescent moieties (e.g., Qdot™ nanoparticles supplied by the Quantum Dot Corporation, Palo Alto, Calif.). General techniques to be used in performing the various immunoassays noted above are known to those of ordinary skill in the art.

For purposes of diagnosis, the antibodies can be labeled with a detectable moiety including but not limited to radioisotopes, fluorescent labels, and various enzyme-substrate labels know in the art. Methods of conjugating labels to an antibody are known in the art.

In some embodiments, antibodies need not be labeled, and the presence thereof can be detected using a labeled antibody which binds to the antibodies of the present disclosure.

The antibodies of the present disclosure can be employed in any known assay method, such as competitive binding assays, direct and indirect sandwich assays, and immunoprecipitation assays. Zola, Monoclonal Antibodies: A Manual of Techniques, pp. 147-158 (CRC Press, Inc. 1987).

The antibodies and polypeptides can also be used for in vivo diagnostic assays, such as in vivo imaging. Generally, the antibody is labeled with a radionuclide (such as ¹¹¹In, ⁹⁹Tc, ¹⁴C, ¹³¹I, ¹²⁵I, or ³H) so that the cells or tissue of interest can be localized in vivo following administration to a subject.

The antibody may also be used as staining reagent in pathology, e.g., using known techniques.

In another aspect, provided herein is a rapid test with quantum dots labeling for a sensitive and quantitative lateral flow immunoassay. Lateral flow immunoassays (LFIA) use specific antibodies to rapidly detect the presence of antigen in test samples. The assay typically can be performed in less than 10 minutes and require no special equipment or highly trained technicians. The manufacturing costs of these tests are also typically very low compared to other platforms. Since the first introduction of LFIA in pregnancy tests, it has been widely used in clinical POC diagnostics and in the drug abuse screening field.

Aside from many technical details in manufacturing a LFIA device, the most important component for a successful LFIA is typically the target specific antibody. In one aspect, the target specific antibody is a llama single domain antibody as described herein. The detection method is also important. Conventional LFIA is an immuno-chromatographic assay using a colloidal gold or latex-labeled antibody for colorimetric detection of targets. These assays are rapid and simple to use, and are most suitable in field screening applications. However, the results are more qualitative in nature and the sensitivity is often limited.

Fluorescent and luminescent labels have been used to improve sensitivity and quantitation range for LFIA. Semiconductor nanocrystals, also known as quantum dots, are a class of light-emitting materials whose electronic characteristics are closely related to the size and shape of the individual crystal. By simply varying the crystal size, quantum dots emit lights in a wide range of wavelengths, or colors that are less prone to overlap than those of organic dyes. A single light source can excite quantum dots of many colors so that multiple targets can be labeled and detected simultaneously. In addition to this multiplexing capability, quantum dots exhibit brilliant colors and long-term photo-stability and are therefore much brighter than organic dyes and retain their glow much longer. Provided here in some embodiments are methods of using quantum dots for developing multiplexed quantitative point-of-care assay devices, for example, devices for quantitative lateral flow assays using quantum dot labeled antibodies to improve the utility of LFIA as a diagnostic platform. For example, a portable QD (quantum dot) reader (e.g., one from Ocean Nanotech, San Diego) can be used at point-of-care locations. In order to further improve the sensitivity of the HCV core antigen LFIA tests, in some aspects, quantum dots are used to label the HCV core antigen specific antibodies, for example, VHH antibodies specific for HCV core antigen.

In some aspects, LFIA test strips are used to test seroconversion panels. In other aspects, methods and devices for rapid detection of HCV core antigen in serum/plasma from patients with acute and chronic HCV infection are provided.

In one aspect, single domain VHH antibodies, including HCV core antigen antibodies, are generated by immunizing llama with multiple antigens. In some aspects, the affinity and specificity of the antibodies are determined. In other aspects, the antibody is expressed in rabbit Fc fusion format for lateral flow assays. In still other aspects, pairing antibodies for sandwich immunoassays are identified are provided. In one aspect, two seroconversion panels are tested in ELISA with one pair of HCV core antigen antibodies. In some embodiments, the antibodies are used to further develop diagnostic ELISA kits and rapid test LFIA devices.

In some embodiments, provided herein are rapid test devices with LFIA using colloidal gold and quantum dots. In one aspect, VHH with rabbit Fc and its application on rapid test devices are used to develop the rapid test devices. First, a conventional LFIA with colloidal gold labeling is constructed, which can provide a quick estimate of specificity and detection limit. Using the quantum dot labeled antibodies, sandwich LFIA strips can be assembled. With the optimized condition and constructed LFIA, HCV seroconversion panels can be tested and compared to ELISA results.

In one embodiment, VHH-rFc antibodies are used in lateral flow immunoassays to detect a small molecule hapten, such as one of about 126 Dalton. In one embodiment, a conventional LFIA with colloidal gold labeling is constructed. The limit of detection typically reaches 10 to 100 ng/ml or lower. By varying the amount of antibody printed on the strip and antibody to gold ratio, a working condition for test strips can be identified. Recombinant HCV core antigen can be tested to determine the LOD of these devices.

In another aspect, using the quantum dot labeled antibodies, LFIA strips can be assembled. The sensitivity of quantum dot labeling is typically ˜100 fold better than those of colloidal gold. Cross linking condition including ratio of antibody to cross linker or QD and overall concentration can be determined. To accommodate the use of these devices under denaturing conditions where regular goat anti-rabbit antibodies fail to bind targets and cannot be used on the control line, a VHH antibody and its target antigen can be used as control. For example, the AG01-BSA antigen can be printed on the control line and labeled AG01 specific VHH-rFC can be sprayed on the conjugate pad with the labeled HCV core antigen antibodies. In one aspect, the AG01 VHH-rFc binds its target in the presence of strong denaturant, and therefore serves as a proper control under this condition.

In one aspect, to construct the LFIA, a nitrocellulose membrane is printed with AG01-BSA at the control line at 1 mg/ml at 10 μl/cm speed. The test line is printed with capture antibody at 1 mg/ml. Purified VHH-rFc (for HCV core antigen and AG01 antibody) is conjugated to colloidal gold or quantum dots at between about 5 and about 50 gig/ml (actual concentration to be optimized individually) and dried on conjugation pads with conjugate-release buffer. HCV core antigen in various concentrations can be tested on assembled test strips. Detection limit and linear range can be determined for each pair of antibodies.

As discussed earlier, the sample from patient blood is typically complicated with anti-HCV antibodies, and actual testing samples need to be denatured to disrupt the antibody-antigen binding and destroy the human antibody present in the sample before performing immuno-tests. In some embodiments, tests of LFIA are performed under various denaturing conditions. The detection limit and sensitivity under each condition can be determined.

### V. Exemplary Embodiments

Among the embodiments provided herein are:

1. A polypeptide comprising:
a) an isolated hepatitis C virus (HCV) core antigen polypeptide comprising the amino acid sequence set forth in SEQ ID NO:1; or
b) an isolated HCV core antigen fragment comprising any one of the amino acid sequences set forth in SEQ ID NOs: 2-11, or any combination thereof, wherein said polypeptide does not comprise a full length natural HCV core antigen.

2. The polypeptide of embodiment 1, which is a part of a fusion polypeptide.

3. The polypeptide of embodiment 2, which further comprises a tag sequence.

4. The polypeptide of any of embodiments 1-3, which comprises or is conjugated to a detectable label.

5. The polypeptide of embodiment 4, wherein the detectable label is a colorimetric, a radioactive, an enzymatic, a luminescent or a fluorescent label.

6. The polypeptide of embodiment 4 or 5, wherein the detectable label is a soluble label or a particle (such as a nanoparticle or a microparticle) or particulate label.

7. The polypeptide of any of embodiments 1-6, which is attached to a solid surface, such as a blot, a membrane, a sheet, a paper, a bead, a particle (such as a nanoparticle or a microparticle), an assay plate, an array, a glass slide, a microtiter, or an ELISA plate.

8. A polynucleotide which encodes the polypeptide of any of embodiments 1-7, or a complimentary strand thereof.

9. The polynucleotide of embodiment 8, which is codon-optimized for expression in a non-human organism or a cell.

10. The polynucleotide of embodiment 9, wherein the organism or cell is a virus, a bacterium, a yeast cell, a plant cell, an insect cell, or a mammalian cell.

11. The polynucleotide of any of embodiments 7-10, wherein the polynucleotide is DNA or RNA.

12. The polynucleotide of embodiment 8, wherein the polynucleotide comprises the nucleotide sequence of SEQ ID NO: 12.

13. A vector comprising the polynucleotide of any of embodiments 8-12.

14. The vector of embodiment 13, wherein the polynucleotide further comprises a promoter sequence.

15. The vector of embodiment 13 or 14, wherein the polynucleotide further encodes a tag sequence.

16. The vector of any of embodiments 13-15, wherein the polynucleotide comprises a poly-A sequence.

17. The vector of any of embodiments 13-16, wherein the polynucleotide comprises a translation termination sequence.

18. A non-human organism or a cell transformed with the vector of any of embodiments 13-17.

19. The non-human organism or cell of embodiment 18, which is a virus, a bacterium, a yeast cell, an insect cell, a plant cell, or a mammalian cell.

20. A method of recombinantly making a polypeptide, which method comprises culturing the organism or cell of embodiment 19, and recovering said polypeptide from said organism or cell.

21. The method of embodiment 20, further comprising isolating the polypeptide, optionally by chromatography.

22. A polypeptide produced by the method of embodiment 20 or 21.

23. The polypeptide of embodiment 22, wherein the polypeptide comprises a native glycosylation pattern.

24. The polypeptide of embodiment 22 or 23, wherein the polypeptide comprises a native phosphorylation pattern.

25. A kit for detecting an antibody that specifically binds to an HCV core antigen polypeptide, which kit comprises, in a container, the polypeptide of any of embodiments 1-7 and 22-24.

26. A method for detecting an antibody that specifically binds to an HCV core antigen polypeptide in a sample, which method comprises contacting the polypeptide of embodiments 1-7 and 22-24 with said sample and detecting a polypeptide-antibody complex formed between the polypeptide and the HCV core antigen polypeptide in the sample to assess the presence, absence and/or amount of the antibody that specifically binds to an HCV core antigen polypeptide in the sample.

27. The method of embodiment 26, wherein the sample is from a mammal.

28. The method of embodiment 27, wherein the mammal is a human.

29. The method of embodiment 27 or 28, wherein the method is used for diagnosis, prognosis, stratification, risk assessment, or treatment monitoring of an HCV infection.

30. The method of any of embodiments 27-29, wherein the sample is selected from the group consisting of a whole blood sample, a serum, a plasma, a urine and a saliva sample.

31. The method of any of embodiments 27-30, wherein the sample is a clinical sample.

32. The method of any of embodiments 27-31, wherein the polypeptide-antibody complex is assessed by a sandwich or competitive assay format, optionally with a binder or antibody.

33. The method of embodiment 32, wherein the binder or antibody is attached to a surface and functions as a capture binder or antibody.

34. The method of embodiment 32 or 33, wherein at least one of the binders or antibodies is labeled.

35. The method of any of embodiments 27-34, wherein the polypeptide-antibody complex is assessed by a format selected from the group consisting of an enzyme-linked immunosorbent assay (ELISA), immunoblotting, immunoprecipitation, radioimmunoassay (RIA), immunostaining, latex agglutination, indirect hemagglutination assay (IHA), complement fixation, indirect immunofluorescent assay (IFA), nephelometry, flow cytometry assay, plasmon resonance assay, chemiluminescence assay, lateral flow immunoassay, μ-capture assay, inhibition assay and avidity assay.

36. The method of any of embodiments 27-34, wherein the polypeptide-antibody complex is assessed in a homogeneous or a heterogeneous assay format.

1a. An isolated camelid antibody that specifically binds to an epitope within an HCV core antigen polypeptide.

2a. The isolated camelid antibody of embodiment 1a, which is derived from a camel, a llama, an alpaca (Vicugna pacos), a vicuña (Vicugna vicugna), or a guanaco (Lama guanicoe).

3a. The isolated camelid antibody of embodiment 2a, wherein the camel is a dromedary camel (Camelus dromedarius), a Bactrian camel (Camelus bactrianus), or a wild Bactrian camel (Camelus ferus).

4a. The isolated camelid antibody of any of embodiments 1a-3a, wherein the antibody is a polyclonal antibody, a monoclonal antibody, an antibody fragment or a single-domain heavy-chain (VHH) antibody.

5a. The isolated camelid antibody of embodiment 4a, wherein the VHH antibody is a llama VHH antibody.

6a. The isolated camelid antibody of any of embodiments 1a-5a, wherein the antibody specifically binds to an epitope within an HCV core antigen polypeptide from a genotype selected from the group consisting of 1, 1a, 1a/1b, 1b, 2, 2a, 2a/2c, 2b, 3a, 3k, 4, 4a, 4a/4c, 4c/4d, 4c/4d/4e, 5/5a, 6a, and 6i.

7a. The isolated camelid antibody of any of embodiments 1a-5a, wherein the antibody specifically binds to an epitope within the polypeptide comprising the amino acid sequence set forth in SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, or SEQ ID NO:11, or any combination thereof.

8a. The isolated camelid antibody of embodiment 7a, which is produced by a process that comprises the steps of:
a) immunizing a camelid with a polypeptide comprising the amino acid sequence set forth in SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, or SEQ ID NO: 11, or any combination thereof; and
b) recovering the antibody from the camelid.

9a. The isolated camelid antibody of embodiment 8a, wherein the camelid is a llama.

10a. The isolated camelid antibody of any of embodiments 1a-9a, wherein the antibody specifically binds to the HCV core antigen polypeptide.

11a. The isolated camelid antibody of any of embodiments 1a-10a, which is a part of a fusion polypeptide.

12a. The isolated camelid antibody of embodiment 11a, wherein the fusion polypeptide comprises a variable region of a camelid antibody and a constant region of a non-camelid antibody.

13a. The isolated camelid antibody of embodiment 12a, wherein the fusion polypeptide comprises a variable region of a llama antibody and a constant region of a non-camelid antibody.

14a. The isolated camelid antibody of embodiment 13a, wherein the fusion polypeptide comprises a variable region of a llama antibody and a constant region of a rabbit antibody.

15a. The isolated camelid antibody of embodiment 14a, wherein the fusion polypeptide is a fusion llama VHH antibody that comprises a variable region of the llama VHH antibody and a Fc region of a rabbit antibody.

16a. The isolated camelid antibody of any of embodiments 1a-15a, which is a humanized antibody.

17a. The isolated camelid antibody of any of embodiments 1a-16a, which is conjugated to a detectable label.

18a. The isolated camelid antibody of embodiment 17a, wherein the detectable label is a colorimetric, a radioactive, an enzymatic, a luminescent or a fluorescent label.

19a. The isolated camelid antibody of embodiment 17a or 18a, wherein the detectable label is a soluble label or a particle (such as a nanoparticle or a microparticle) or particulate label.

20a. The isolated camelid antibody of any of embodiments 1a-19a, which is attached to a solid surface, such as a blot, a membrane, a sheet, a paper, a bead, a particle (such as a nanoparticle or a microparticle), an assay plate, an array, a glass slide, a microtiter, or an ELISA plate.

21a. A method for detecting an HCV core antigen polypeptide in a sample, which method comprises contacting the HCV core antigen polypeptide in the sample with an isolated camelid antibody of any of embodiments 1a-20a, and detecting a polypeptide-antibody complex formed between the HCV core antigen polypeptide in the sample and the isolated camelid antibody to assess the presence, absence and/or amount of the HCV core antigen polypeptide in the sample.

22a. The method of embodiment 21a, wherein the sample is from a subject, e.g., a mammal.

23a. The method of embodiment 22a, wherein the mammal is a human.

24a. The method of any of embodiments 21a-23a, wherein the method is used for diagnosis, prognosis, stratification, risk assessment, or treatment monitoring of an HCV infection.

25a. The method of any of embodiments 21a-23a, wherein the method is used for identifying HCV infection in a seronegative mammal, identifying a seropositive mammal that is actively infected with HCV, or for monitoring an anti-HCV therapy.

26a. The method of any of embodiments 21a-25a, wherein the sample is selected from the group consisting of a whole blood sample, a serum, a plasma, a urine and a saliva sample.

27a. The method of any of embodiments 21a-26a, wherein the sample is a clinical sample.

28a. The method of any of embodiments 21a-27a, wherein the polypeptide-antibody complex is assessed by a sandwich or competitive assay format.

29a. The method of embodiment 28a, wherein the camelid antibody is attached to a surface and functions as a capture antibody.

30a. The method of embodiment 28a, wherein the camelid antibody is labeled.

31a. The method of embodiment 28a, wherein the polypeptide-antibody complex is assessed by a sandwich assay format that uses two camelid antibodies, one being a capture antibody and the other being a labeled antibody.

32a. The method of any of embodiments 21a-31a, wherein the polypeptide-antibody complex is assessed by a format selected from the group consisting of an enzyme-linked immunosorbent assay (ELISA), immunoblotting, immunoprecipitation, radioimmunoassay (RIA), immunostaining, latex agglutination, indirect hemagglutination assay (IHA), complement fixation, indirect immunofluorescent assay (IFA), nephelometry, flow cytometry assay, plasmon resonance assay, chemiluminescence assay, lateral flow immunoassay, μ-capture assay, inhibition assay and avidity assay.

33a. The method of any of embodiments 21a-31a, wherein the polypeptide-antibody complex is assessed in a homogeneous or a heterogeneous assay format.

34a. The method of any of embodiments 21a-33a, which further comprises disassociating the HCV core antigen polypeptide in the sample from an antibody of the subject to be tested.

35a. The method of embodiment 34a, wherein the HCV core antigen polypeptide in the sample is disassociated from the antibody of the subject to be tested by changing the pH of the sample to be 4 or lower, or to be 9 or higher, by treating the sample with a protein denaturing agent, and/or by heating the sample to between about 35° C. and about 95° C., preferably to between about 45° C. and about 70° C., concurrently with or before contacting the sample with the camelid antibody.

36a. The method of embodiment 35a, wherein the protein denaturing agent is guanidine hydrochloride (e.g., about 1 M to about 6 M), guanidinium thiocyanate (e.g., about 1 M to about 6 M), SDS (e.g., about 0.1% to about 2%), □-mercaptoethanol, DTT or other reducing agent for disulfide bond disruption at various concentrations, or urea (e.g., about 2 M to about 8 M), or any combination thereof.

37a. The method of embodiment 35a or 36a, which further comprises adjusting the pH of the sample to between about 6 and about 8, and/or removing the protein denaturing agent concurrently with or before contacting the sample with the camelid antibody.

38a. The method of embodiment 35a or 36a, wherein the camelid antibody is a camelid VHH antibody, and the sample is contacted with the camelid VHH antibody at a pH that is at 4 or lower, or at 9 or higher, and/or in the presence of the protein denaturing agent.

39a. The method of embodiment 38a, wherein the camelid VHH antibody is a llama VHH antibody.

40a. A kit for detecting an HCV core antigen polypeptide, which kit comprises, in a container, an isolated camelid antibody of any of embodiments 1a-20a.

41a. A lateral flow device comprising a matrix that comprises an isolated camelid antibody of any of embodiments 1a-20a immobilized on a test site on the matrix downstream from a sample application site on the matrix.

42a. The lateral flow device of embodiment 41a, which further comprises a labeled camelid antibody of any of embodiments 1a-20a on the matrix upstream from the test site, said labeled camelid antibody being capable of moved by a liquid sample and/or a further liquid to the test site and/or a control site to generate a detectable signal.

43a. A method for detecting an HCV core antigen polypeptide in a liquid sample, which method comprises:
a) contacting a liquid sample with the lateral flow device of embodiment 41a or 42a, wherein the liquid sample is applied to a site of the lateral flow device upstream of the test site;
b) transporting an HCV core antigen polypeptide, if present in the liquid sample, and a labeled camelid antibody of any of embodiments 1a-20a to the test site; and
c) assessing the presence, absence, and/or amount of a signal generated by the labeled camelid antibody at the test site to determining the presence, absence and/or amount of the HCV core antigen polypeptide in the liquid sample.

1b. A method for detecting an analyte in a sample from a subject, which method comprises:
a) disassociating an analyte in a sample from a subject that is bound to an antibody of the subject from the antibody of the subject by changing the pH of the sample to be 4 or lower, or to be 9 or higher, by treating the sample with a protein denaturing agent, and/or by heating the sample to between about 35° C. and about 95° C., preferably to between about 45° C. and about 70° C.;
b) contacting the analyte disassociated from the antibody of the subject with a camelid VHH antibody at a pH that is at 4 or lower, or at 9 or higher, and/or in the presence of the protein denaturing agent, and/or at a temperature of about 35° C. and about 95° C., preferably about 45° C. and about 70° C., and detecting an analyte-antibody complex formed between the disassociated analyte and the camelid antibody to assess the presence, absence and/or amount of the analyte in the sample.

2b. The method of embodiment 1b, wherein the analyte is selected from the group consisting of a cell, a cellular organelle, a virus, a molecule and an aggregate or complex thereof.

3b. The method of embodiment 2b, wherein the cell is selected from the group consisting of an animal cell, a plant cell, a fungus cell, a bacterium cell, a recombinant cell and a cultured cell.

4b. The method of embodiment 2b, wherein the cellular organelle is selected from the group consisting of a nucleus, a mitochondrion, a chloroplast, a ribosome, an ER, a Golgi apparatus, a lysosome, a proteasome, a secretory vesicle, a vacuole and a microsome.

5b. The method of embodiment 2b, wherein the molecule is selected from the group consisting of an inorganic molecule, an organic molecule and a complex thereof.

6b. The method of embodiment 5b, wherein the inorganic molecule is an ion selected from the group consisting of a sodium, a potassium, a magnesium, a calcium, a chlorine, an iron, a copper, a zinc, a manganese, a cobalt, an iodine, a molybdenum, a vanadium, a nickel, a chromium, a fluorine, a silicon, a tin, a boron and an arsenic ion.

7b. The method of embodiment 5b, wherein the organic molecule is selected from the group consisting of an amino acid, a peptide, a protein, a polypeptide, a nucleoside, a nucleotide, an oligonucleotide, a nucleic acid, a polynucleotide, a vitamin, a monosaccharide, an oligosaccharide, a polysaccharide a carbohydrate, a lipid and a complex thereof.

8b. The method of any of embodiments 1b-7b, wherein the analyte is a marker for a disease, disorder or infection.

9b. The method of embodiment 8b, wherein the method is used for diagnosis, prognosis, stratification, risk assessment, or treatment monitoring of the disease, disorder or infection.

10b. The method of embodiment 8b, wherein the analyte is a marker for is bacterial or viral infection.

11b. The method of embodiment 10b, wherein the analyte is a marker for HCV infection.

12b. The method of embodiment 11b, wherein the analyte is an HCV polypeptide.

13b. The method of embodiment 12b, wherein the HCV polypeptide is an HCV core antigen polypeptide.

14b. The method of any of embodiments 11b-13b, wherein the method is used for diagnosis, prognosis, stratification, risk assessment, or treatment monitoring of an HCV infection.

15b. The method of any of embodiments 11b-13b, wherein the method is used for identifying HCV infection in a seronegative mammal, identifying a seropositive mammal that is actively infected with HCV, or for monitoring an anti-HCV therapy.

16b. The method of any of embodiments 1b-15b, wherein the subject is a mammal.

17b. The method of embodiment 16b, wherein the mammal is a human.

18b. The method of any of embodiments 1b-17b, wherein the sample is selected from the group consisting of a whole blood sample, a serum, a plasma, a urine and a saliva sample.

19b. The method of any of embodiments 1b-18b, wherein the sample is a clinical sample.

20b. The method of any of embodiments 1b-19b, wherein the analyte is disassociated from the antibody of the subject by changing the pH of the sample to be 4 or lower, or wherein the analyte is disassociated from the antibody of the subject by changing the pH of the sample to be 9 or higher.

21b. The method of any of embodiments 1b-19b, wherein the analyte is disassociated from the antibody of the subject by heating the sample to between about 35° C. and about 95° C., preferably to between about 45° C. and about 70° C.

22b. The method of any of embodiments 1b-19b, wherein the analyte is disassociated from the antibody of the subject by treating the sample with a protein denaturing agent.

23b. The method of embodiment 16b, wherein the protein denaturing agent is guanidine hydrochloride (e.g., about 1 M to about 6 M), guanidinium thiocyanate (e.g., about 1 M to about 6 M), SDS (e.g., about 0.1% to about 2%), □-mercaptoethanol, DTT or other reducing agent for disulfide bond disruption at various concentrations, or urea (e.g., about 2 M to about 8 M), or any combination thereof.

24b. The method of any of embodiments 1b-19b, wherein the analyte is disassociated from the antibody of the subject by changing the pH of the sample to be 4 or lower, by treating the sample with a protein denaturing agent, and/or by heating the sample to between about 35° C. and about 95° C., preferably to between about 45° C. and about 70° C.

25b. The method of any of embodiments 1b-19b, wherein the analyte is disassociated from the antibody of the subject by changing the pH of the sample to be 9 or higher, by treating the sample with a protein denaturing agent, and/or by heating the sample to between about 35° C. and about 95° C., preferably to between about 45° C. and about 70° C.

26b. The method of any of embodiments 1b-25b, wherein the analyte disassociated from the antibody of the subject is contacted with the camelid VHH antibody at a pH that is at 4 or lower, or wherein the analyte disassociated from the antibody of the subject is contacted with the camelid VHH antibody at pH that is at 9 or higher.

27b. The method of any of embodiments 1b-25b, wherein the analyte disassociated from the antibody of the subject is contacted with the camelid VHH antibody at a temperature of about 35° C. and about 95° C., preferably about 45° C. and about 70° C.

28b. The method of any of embodiments 1b-25b, wherein the analyte disassociated from the antibody of the subject is contacted with the camelid VHH antibody in the presence of the protein denaturing agent.

29b. The method of embodiment 28b, wherein the protein denaturing agent is guanidine hydrochloride (e.g., about 1 M to about 6 M), guanidinium thiocyanate (e.g., about 1 M to about 6 M), SDS (e.g., about 0.1% to about 2%), β-mercaptoethanol, DTT or other reducing agent for disulfide bond disruption at various concentrations, or urea (e.g., about 2 M to about 8 M), or any combination thereof.

30b. The method of any of embodiments 1b-25b, wherein the analyte disassociated from the antibody of the subject is contacted with the camelid VHH antibody at a pH that is at 4 or lower and in the presence of the protein denaturing agent.

31b. The method of any of embodiments 1b-25b, wherein the analyte disassociated from the antibody of the subject is contacted with the camelid VHH antibody at pH that is at 9 or higher and in the presence of the protein denaturing agent.

32b. The method of any of embodiments 1b-31b, wherein the camelid VHH antibody is a llama VHH antibody.

33b. The camelid antibody of embodiment 32b, wherein the llama VHH antibody is a fusion llama VHH antibody that comprises a variable region of the llama VHH antibody and a constant region of a rabbit antibody.

34b. The method of any of embodiments 1b-33b, wherein the analyte-antibody complex is assessed by a sandwich or competitive assay format.

35b. The method of embodiment 34b, wherein the camelid antibody is attached to a surface and functions as a capture antibody.

36b. The method of embodiment 34b, wherein the camelid antibody is labeled.

37b. The method of embodiment 34b, wherein the analyte-antibody complex is assessed by a sandwich assay format that uses two camelid antibodies, one being a capture antibody and the other being a labeled antibody.

38b. The method of any of embodiments 1b-37b, wherein the analyte-antibody complex is assessed by a format selected from the group consisting of an enzyme-linked immunosorbent assay (ELISA), immunoblotting, immunoprecipitation, radioimmunoassay (RIA), immunostaining, latex agglutination, indirect hemagglutination assay (IHA), complement fixation, indirect immunofluorescent assay (IFA), nephelometry, flow cytometry assay, plasmon resonance assay, chemiluminescence assay, lateral flow immunoassay, μ-capture assay, inhibition assay and avidity assay.

39b. The method of any of embodiments 1b-37b, wherein the analyte-antibody complex is assessed in a homogeneous or a heterogeneous assay format.

40b. The method of embodiment 37b, wherein the analyte-antibody complex is assessed by a lateral flow sandwich assay format that uses two camelid antibodies, one being a capture antibody and the other being a labeled antibody.

41b. The method of embodiment 40b, wherein the labeled antibody is labeled with a particle (such as a nanoparticle or a microparticle) or particulate label.

42b. The method of any of embodiments 1b-41b, wherein the steps a) and b) are conducted concurrently.

43b. The method of any of embodiments 1b-41b, wherein the step a) is conducted before the step b).

44b. The method of any of embodiments 1b-43b, which is conducted to assess the presence or absence of the analyte in the sample.

45b. The method of any of embodiments 1b-43b, which is conducted to assess the amount of the analyte in the sample.

### EXAMPLES

### Example 1: Llama Immunization

Provided in this example is a method for isolating high affinity VHH antibodies from immunized llamas through in vitro screening. Using this method, multiple VHH antibodies for small molecule haptens were isolated. One of the highest affinity antibody had a kD of 60 pM. Many of these VHH antibodies had kD in the ˜100 μM range as determined by ELISA.

In one experiment, five small molecule chemicals were custom synthesized/modified by Annova Chem (San Diego, Calif.) to have a carboxyl group for conjugation purposes. The chemicals were designated AG01, AG02, AG03, AG04, and AG05, and their molecular structures are provided in Table 2.

TABLE 2
List of small molecule chemicals used in llama immunization.

| Antigen ID | Compound Name | Structure |
|---|---|---|
| AG01 | 2,5-dioxopyrrolidin-1-yl 6-((4,6-diamino- 1,3,5-triazin-2-yl)amino)hexanoate | 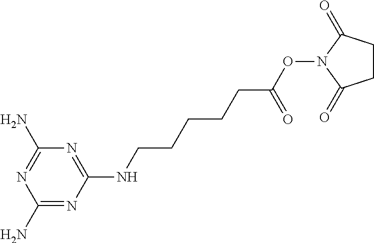 [SMILES: Nc1nc(N)nc(NCCCCCC(=O)ON2C(=O)CCC2=O)n1] |
| AG02 | 2,5-dioxopyrrolidin-1-yl 5-((4-(2-(tert- butylamino)-1-hydroxyethyl)-2,6- dichlorophenyl)amino)-5-oxopentanoate | 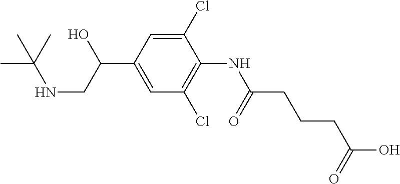 [SMILES: CC(C)(C)NCC(O)c1cc(Cl)c(NC(=O)CCCC(=O)O)c(Cl)c1] |
| AG03 | 2,5-dioxopyrrolidin-1-yl 4-((5-(2-(tert- butylamino)-1-hydroxyethyl)-2-hydroxy- 3- (hydroxymethyl)phenyl)diazenyl)benzoate | 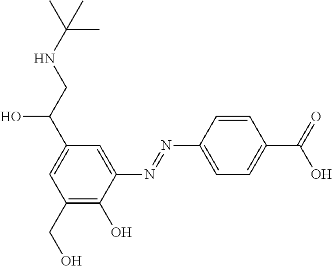 [SMILES: CC(C)(C)NCC(O)c1cc(CO)c(O)c(/N=N/c2ccc(C(=O)O)cc2)c1] |
| AG04 | 2,5-dioxopyrrolidin-1-yl (4-(3-((2- hydroxy-2-(4- hydroxyphenyl)ethyl)amino)butyl)phenyl) glutarate | 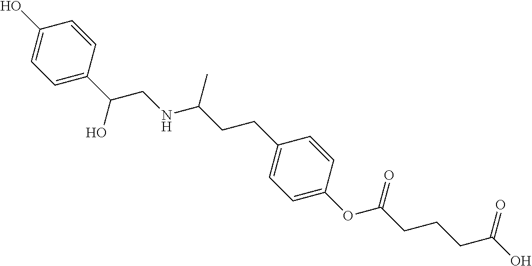 [SMILES: CC(CCc1ccc(OC(=O)CCCC(=O)O)cc1)NCC(O)c1ccc(O)cc1] |
| AG05 | Aflatoxin B1 oxime OSu | 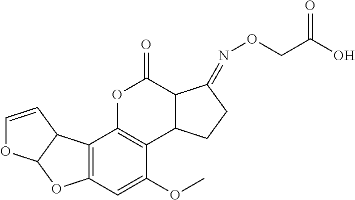 [SMILES: COc1cc2c(c3c1C1CC/C(=N\OCC(=O)O)C1C(=O)O3)C1C=COC1O2] |

Each chemical was dissolved in DMSO at a concentration of 50 mg/ml and diluted to 20 mg/ml with MES buffer (0.1 M, pH 6.4). KLH were reconstituted to 20 mg/ml prior to use. An equimolar ratio of NHS and EDC was dissolved in MES buffer and added to the conjugation reactions. For each chemical, 1 mg was conjugated to 1 mg of KLH. Similarly, each chemical was also conjugated to BSA and Biotin (amine-PEG11-Bioitin, Cat #26136, Thermofisher Scientific). All five KLH conjugated chemicals were mixed at equal amounts and the final concentration was adjusted to 2 mg/ml of KLH prior to immunization.

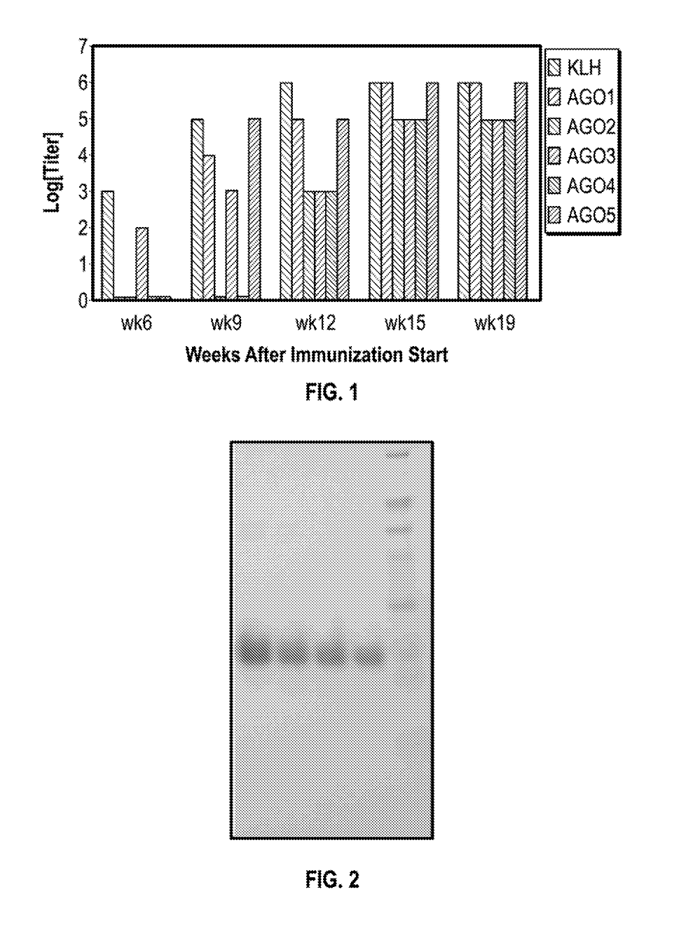

A 4-year old female llama was immunized with the mixed antigen. Antibody titers were monitored by ELISA using biotinylated antigens and streptavidin coated 96-well plates with test anti-sera taken on days as indicated in FIG. 1.

FIG. 1 shows serum ELISA titers for various antigens used in this example. Test bleeds were collected on the days indicated. Serum was serial diluted by a factor of 10 and ELISA was performed using biotinylated antigens and streptavidin coated 96-well plates. Positive titers were qualitatively determined when the signal was over 2 fold above background (biotin only, no antigen).

AG01 and AG05 were able to elicit a strong immune response to yield ELISA titers of up to 106, which is similar to the level of the KLH response. The other three antigens (AG02, AG03, and AG04) yielded lower immune responses, but still were at titers of 105. These titers are all significantly higher than what is usually seen in rabbits, which normally yield titers in the 104 ranges. See Ge et al., Pooled protein immunization for identification of cell surface antigens in Streptococcus sanguinis, PLoS One, 2010, 5(7):e11666. These data suggest that llama is a better host for generating higher affinity antibodies to small molecules.

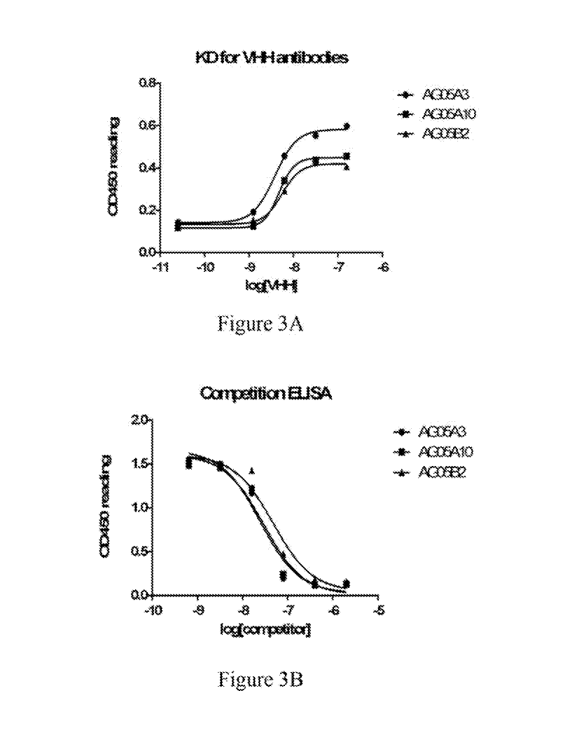

Genes of high affinity antibodies can be cloned and expressed from llama peripheral blood cells. Peripheral blood cells were collected from the immunized llama. Multiple VHH antibodies specific to each antigen were cloned. Using blood cells from week 6 (42 days into the immunization), two specific VHH clones were obtained for KLH; using blood cells from week 9 (63 days), multiple VHH clones at various affinity were obtained for AG05. Other antigen specific VHH clones were obtained for AG01, AG02, AG03, and AG04 from blood cells at week 19. VHH antibodies were purified at milligram scale to >95% purity (FIG. 2). Affinity and specificity of each antibody were determined by direct and competition ELISA. Many of the selected VHHs had apparent kD of about 100 pM (FIG. 3A), and were specifically competed by the cognate antigen (FIG. 3B).

FIG. 2 shows coomassie blue stained SDS-PAGE gel (4-20% gradient gel). The left four lanes were loaded with 3-5 μg of purified VHH from different clones, the right lane was loaded with NEB pre-stained color plus protein marker. The 23 kDa maker is indicated. The calculated molecular weight of VHH produced from the expression system was 21 kDa.

FIG. 3A shows the results of direct ELISA. The affinity of each VHH antibody was determined through the capture of purified VHH antibodies at serial diluted concentrations by biotinylated antigen bound to streptavidin coated plates. Detection was performed with HRP-labeled goat anti-llama antibody. FIG. 3B shows the results of competition/Inhibition ELISA. The biotinylated antigen was captured on streptavidin coated plates. The unmodified antigen (AG05) was serial diluted with binding buffer containing 50 nm of each VHH antibody and added to the plate. After incubation and washing, bound VHH antibody was detected with HRP-labeled goat anti-llama antibody. AG05 inhibited the binding of VHH to the antigen captured on the plate. AG05A3, AG05A10 and AG05B2 were three VHH clones obtained for the AG05 antigen.

### Example 2: Use of VHH Antibodies in Rapid Detection Platforms

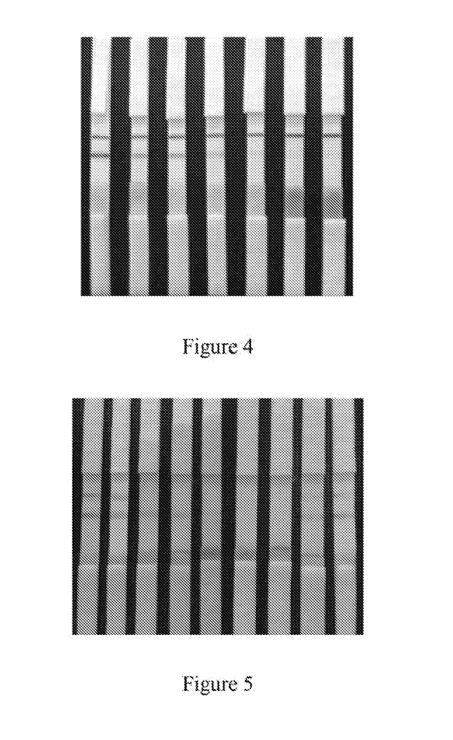

Fusion proteins of VHH antibodies were made with rabbit Fc domains (VHH-rFc). The antibodies were expressed/purified, and can be detected with widely available secondary antibodies to rabbit IgG. These antibodies were successfully used to produce LFIA devices (FIG. 4). The detection limit of AG01 with these LFIA was at 250 ng/ml without any optimization. These data demonstrate that VHH antibodies can be successful used to produce LFIA devices with high sensitivity to small molecules.

FIG. 4 shows a competition lateral flow immunoassay using VHH-rFc fusion antibody for AG01. The control line was printed with goat anti-rabbit antibody. The test line was printed with AG01-BSA conjugate. The VHH-rFc fusion antibody for AG01 was labeled with colloidal gold and printed on the conjugation pad. Samples containing serial diluted AG01 were applied to each strip (1, no AG01; 2, 0.25 ng/ml; 3. 2.5 ng/ml, 4. 25 ng/ml; 5. 250 ng/ml; 6. 2.5 μg/ml; 7.25 μg/ml). When AG01 was not present in the sample (strip 1), the labeled VHH-rFc antibody was captured by the AG01-BSA on the test line therefore a visible line appeared. When AG01 was present in the sample at high concentrations (strip 7), the free AG01 in the sample competed with the AG01-BSA on the test line for the binding of VHH-rFc, therefore the test line was invisible.

It was further demonstrated that the VHH antibody bound to its antigen in the presence of 3M Guanidine HCl or 0.2% SDS (FIG. 5). However, the control line disappeared at lower concentrations (2M) of the denaturants.

FIG. 5 shows the results of a lateral flow assays with guanidine hydrochloride and SDS in the sample buffer. Test lines were printed with the AG01-BSA conjugate, and control lines were printed with a goat anti-rabbit polyclonal antibody. Conjugate pads were sprayed with AG01 specific VHH-rFc with colloidal gold conjugate. Strip 1: PBS; Strip2: 1M Guanidine HCl; Strip3: 2M Guanidine HCl; Strip 4: 3 M Guanidine HCl; Strip 5: 5M Guanidine HCl; Strip 6: 1% SDS; Strip 7: 0.5% SDS; Strip 8: 0.2% SDS; and Strip 9: 0.1% SDS.

The buffer for Guanidine HCl contained phenol red as pH indicator, which caused the strip to show an orange color.

These data demonstrate that the VHH antibodies are applicable on rapid diagnostic tests for small molecules in lateral flow immunoassay format, and the binding of antigen to VHH antibodies are more resistant to high concentrations of strong denaturants. The lateral flow assays fabricated with VHH antibodies can be performed under denaturing conditions.

### Example 3: Quantitative LFIA with Quantum Dots (OD) Technology

Conventional lateral flow assays use colloidal gold or latex beads to conjugate antibodies for colorimetric detection and are mostly qualitative. Fluorescent and luminescent labels have been used to improve sensitivity. Semiconductor nanocrystals, also known as quantum dots, are a class of light emitting materials whose electronic characteristics are closely related to the size and shape of the individual crystal. By simply varying the crystal size, quantum dots emit light in a wide range of wavelengths, or colors that are less prone to overlap than those of organic dyes. A single light source can excite quantum dots of many colors so that multiple targets can be labeled and detected simultaneously. In addition to this multiplexing capability, quantum dots exhibit brilliant colors and long-term photostability and are therefore much brighter than organic dyes and retain their glow much longer. For these reasons, quantum dots are good choices for developing multiplexed quantitative point-of-care assay devices.

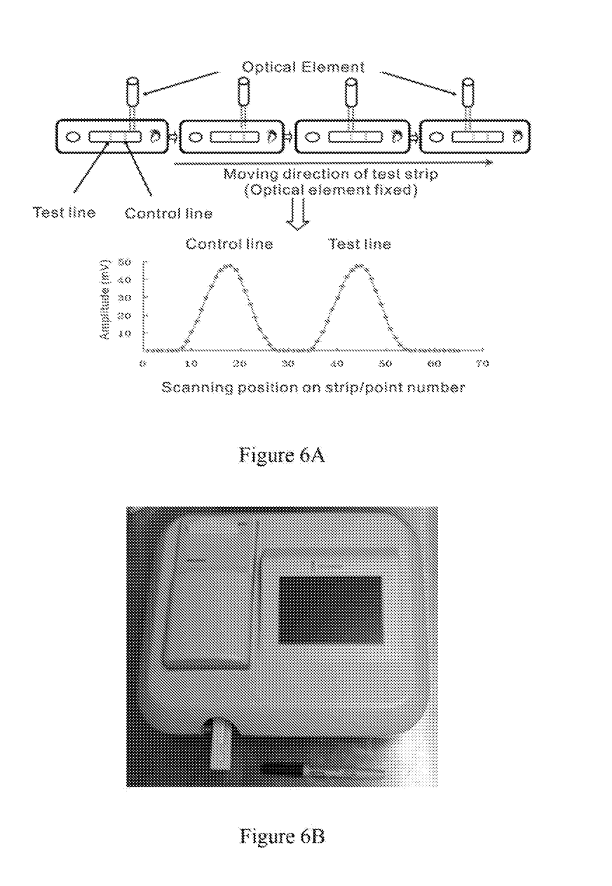

A portable lateral flow reader (Ocean Nanotech) is used for measuring several small molecules with lateral flow assays. FIGS. 6A and 6B shows quantitative LFIA with quantum dots labeled antibodies. FIG. 6A shows an illustration of a test strip and optical read-out. FIG. 6B shows a portable QD reader.

The portable QD-Analyzer has a data microchip for data processing, a LCD touch screen panel for user input and data display, and is equipped with a printer (FIG. 6B). The QD-Analyzer quantitatively measures the fluorescent signals generated by QD. It is self-contained, lightweight, easy to use, and takes <1 minute for strip reading and data processing. Ocean Nanotech has successfully demonstrated a Quantum Dot based strip for folic acid (FA) detection at 1 ng/mL using anti-folate antibodies, and achieved linearity at 0.5-80 ng/mL for folate. This suggests that QD labeled LFIA can be successfully used in quantitative assays of target antigens.

### Example 4: Antigen Preparation and Animal Immunization

In this example, the animal immunization was initiated with a commercially available HCV core antigen, which was made with a β-galactosidase (β-gal) fusion protein (β-gal-core-192). A core protein made with GST fusion tag and the 2-119 amino acid of the HCV core (GST-core-119) was obtained. Since rabbits usually produce faster immune response than llamas do, a rabbit was immunized in parallel to help predicting the course of progress. The animals made most of the antibodies against the β-gal part of the protein, rather than for the HCV core antigen.

Therefore, the full length HCV core antigen was expressed and purified with a poly-Histidine tag (His-core) in an E. coli expression vector, and several milligrams of the protein were purified to over 90% purity.

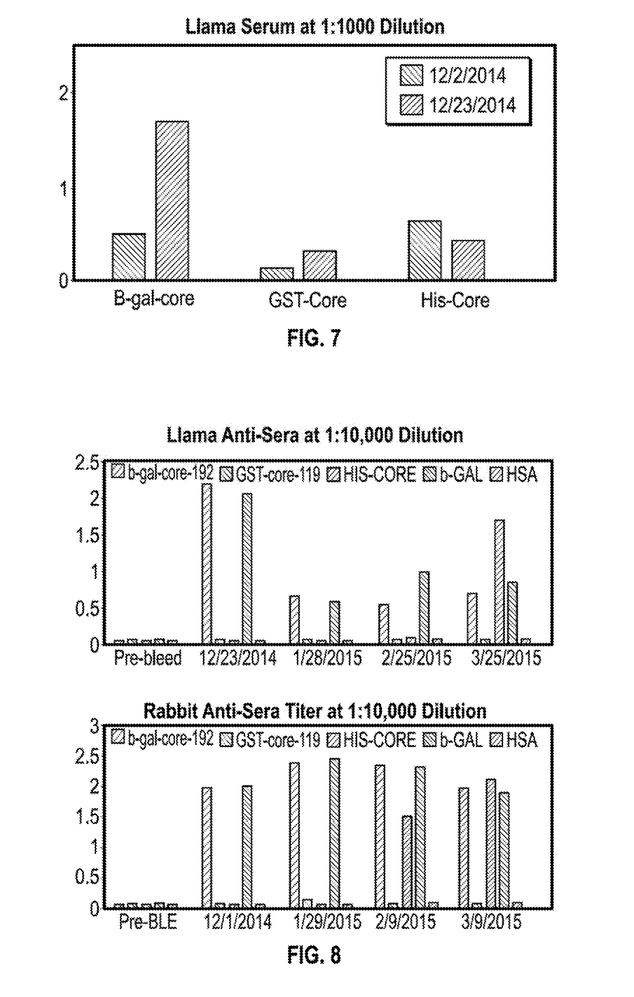

The purified full length HCV core was used to boost the llama and the rabbit. The first positive anti-sera titer for HCV core antigen were observed after 2.5 months of immunization and boosting. The antibody library construction for isolating single domain antibodies were then started (FIG. 7). Meanwhile, boosting of the llama and the rabbit was continued with the core antigen, and the anti-sera titers continued to increase (FIG. 8).

FIG. 7 shows a chart for ELISA results at 1:1000 of serum dilution, showing first positive titer. The data was corrected with pre-bleed signals.

FIG. 8 shows the anti-sera titer for the llama (left) and the rabbit (right), showing ELISA results at 1:10,000 dilution.

The initial bleed had a strong reaction with the β-Gal-Core-192 and the 13-Galactosidase protein, but not much reaction with the GST-core-119 or the His-Core, indicating majority of the antibodies produced were against β-galactosidase. Then immunization with the His-core protein was used, and the anti-sera titer in both rabbit and llama showed strong reactions with the His-core protein while the activity against β-gal decreased. Typically, boosting with the His-core protein will be continued until the anti-sera titer reaches a plateau when fresh blood will be isolated for cloning of highly specific single domain antibodies.

### Example 5: Generating Single Domain Antibodies for HCV Core Antigen

In this example, about 500 ml of blood was collected from the immunized llama, from which 5×10⁸ PBMC cells were isolated and lysed in RNA lysis buffer.

VHH Antibody Library Construction:

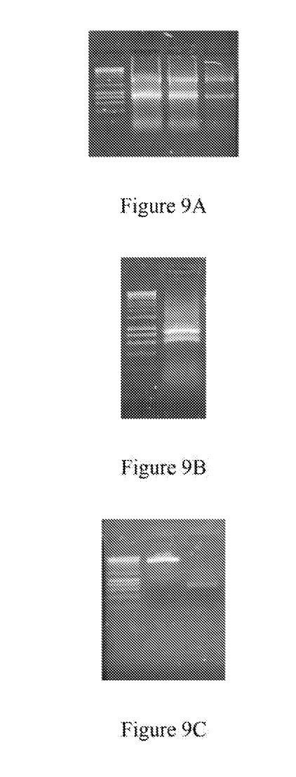

Total RNA was purified from the PBMC cells of the immunized llama (FIG. 9A).

RT-PCR was performed to amplify the variable regions of the heavy chain Ig cDNA, which includes both VH (conventional heavy chain) and VHH (single domain heavy chain only) forms. With our PCR primers the predicted sizes are ˜800 bp for the VH and ˜600 bp for the VHH (FIG. 9B). The two DNA products were separated on an agarose gel after electrophoresis and the 600 bp products were used as template for further PCR and cloning into the phage display vector (pADL20c, from Antibody Design Labs, San Diego) (FIG. 9C). A library of 1×10⁷ independent clones were obtained. 10 clones were randomly picked from the library and DNA was sequenced. All clones contain predicted VHH antibody inserts with correct reading frame.

FIG. 9A shows the total RNA isolated from PBMC cells. FIG. 9B shows PCR product for VH and VHH. FIG. 9C shows DNA prior to library ligation, vector (pADL20c, digested with BglI), insert, digested with sfiI.

Anti-Core VHH Antibody Clone Isolation:

11 candidate clones were identified from initial screenings. Sequence analysis of all clones was performed and relative specificity were determined. Seven of the clones had more diverse sequences throughout the coding region, two of which had only 11 amino acid difference, apparently originated from the same VDJ recombination followed by hypermutation. One of the clones (clone L6A5B2) from the second screening shared identical DNA sequence to clone L4F 11 which was obtained from the first screening, indicating enrichment of selected clones through the screening process.

Affinity Determination of Selected VHH Antibody Clones:

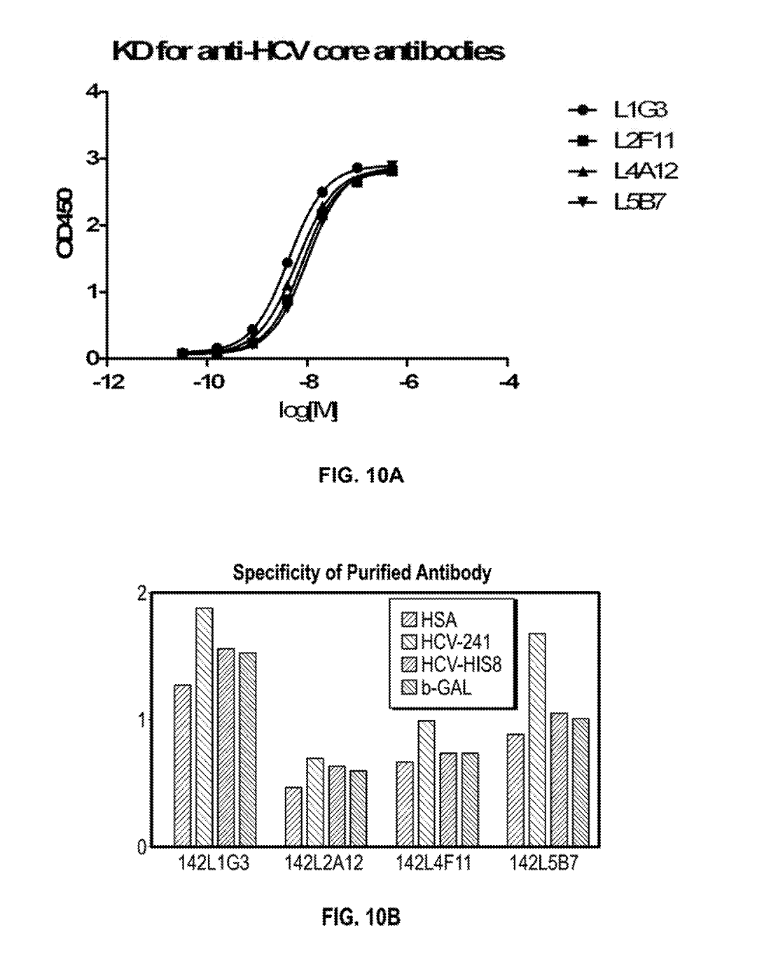

Genes of four VHH antibodies were subcloned to an expression vector for rabbit Fc fusion antibody production. The binding affinity of each antibody to the HCV core protein were determined by ELISA. All four antibodies are showing kD of 4-10 nM (FIG. 10A).

FIGS. 10A and 10B show affinity and specificity of the purified antibodies.

The specificity of each purified antibodies was tested using human serum albumin (HSA), β-galactosidase and two different form of expressed HCV core protein. All four clones showed higher binding affinity to the β-gal-Core-192 (HCV-241) protein that was used for immunization. However, all four antibodies also showed significant binding signals to the β-gal protein (FIG. 10B). This is not surprising because the antibody genes were isolated from the llama when anti-serum just started showing positive titer. On the other hand, the affinity was in the nanomolar range at such early stage.

Finding Antibody Pairs for Sandwich ELISA:

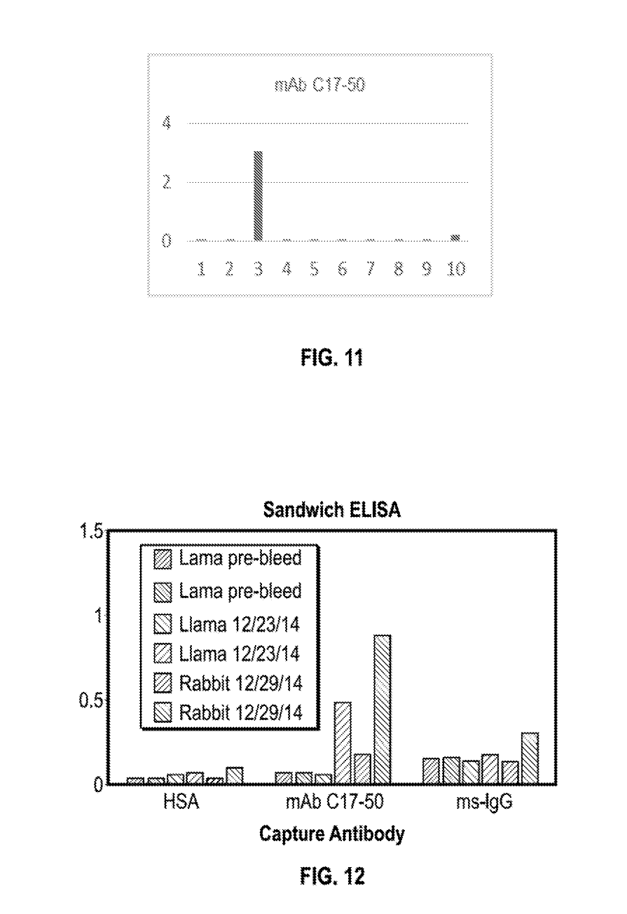

In order to further evaluate the antibodies produced by the llama, pairing antibodies were identified to perform sandwich ELISA to detect the HCV core protein. A mouse monoclonal antibody (clone C7-50) was obtained from Abcam and its binding epitope was tested (FIG. 11) for possible pairing with HCV core antigen antibodies.

FIG. 11 shows binding epitope of the monoclonal antibody C7-50. The ELISA plate was coated with 10 peptides covering the sequences throughout the core protein. The antibody was allowed to bind on the peptides and then detected with HRP-goat anti-mouse antibody. The antibody bound peptide #3 with high specificity.

The antibody C7-50 was used to pair with the anti-serum from the llama or the rabbit in sandwich ELISA to detect the HCV-core protein. The monoclonal antibody C7-50 was able to pair with anti-serum from either the llama or the rabbit (FIG. 12). The mAb C7-50 did not react with the purified core protein HIS-core. One of the possible reasons is that the His-core protein might have different confirmation/structure from the 3-Gal-core-192: the His-core was expressed in the periplasm of the E. Coli and was in native soluble form, while the 3-Gal-core-192 was from inclusion bodies which were denatured in 8M urea. The mAb C7-50 was also generated with a denatured form of the core protein (GST fusion). Whether the antibodies generated with the His-core protein would react with the core protein in patient serum will be tested.

FIG. 12 shows the results of sandwich ELISA. The plate was coated with HSA, antibody C7-50 or mouse IgG. The protein β-Gal-Core-192 was captured, and detected with llama or rabbit anti-serum collected on different date. The antibody C7-50 was able to capture the β-Gal-Core-192 protein, which was then detected with the rabbit (12/29/14) or llama (12/23/14) anti-sera.

The detection limit and sensitivity of the sandwich ELISA were determined using the β-Gal-core-192 protein. Table 3 shows ELISA results for detecting β-gal-192 core with antibody pair of mAb C7-50 and the llama anti-serum. The plate was coated with mouse monoclonal antibody C7-50 (Abcam), the protein β-gal-192 core was serial diluted from 125 g/ml to 8 ng/ml and captured by the antibody. The llama serum was applied and detected with HRP-goat-anti-llama antibody.

TABLE 2
ELISA results for detecting β-gal-192 core with antibody pair
of mAb C7-50 and the llama anti-serum.

| ng/ml antigen | OD450 |
|---|---|
| 125000 | 2.2713 |
| 25000 | 2.1756 |
| 5000 | 1.7355 |
| 1000 | 0.4936 |
| 200 | 0.2999 |
| 40 | 0.214 |
| 8 | 0.2205 |
| 0 | 0.1806 |

The detection limit for β-gal-192 core was at 40 ng/ml or below in this experiment.

### Example 6: Lateral Flow Immunoassays Detecting β-Gal-192 Core

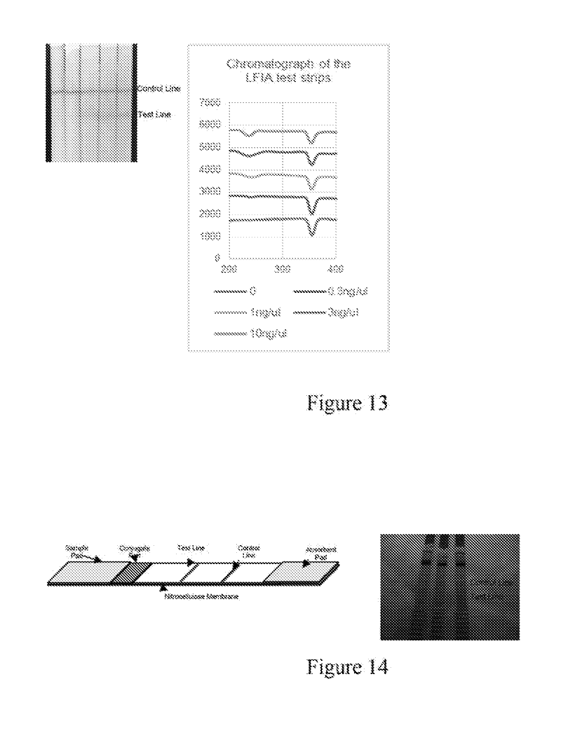

The pairs of antibodies were tested to determine if they could detect the core antigen in LFIA. LFIA test strips were constructed with purified rabbit antibody on the test line, and monoclonal antibody C7-50 was labeled with colloidal gold. Because the rabbit antibody was purified with protein A affinity chromatography, the specific antibodies are only a small fraction of the total purified protein. A higher concentration and larger volume of protein for the test line was applied to capture the antigen. These resulted the test line to appear not as sharp as the control line, which could be resolved when pure specific antibodies are used in production. Nonetheless, the test strips could detect the core antigen at 0.3 ng/μl (FIG. 13). The test strips were also scanned with a LFIA reader from Qiagen (portable ESEQuant lateral flow reader).

FIG. 13 shows LFIA results for detecting β-gal-192 core with antibody pair of mAb C7-50 and the llama anti-serum. On the test strips, the purified rabbit antibody was printed on the test line. The control line was printed with a goat anti-mouse IgG. Monoclonal antibody C7-50 was labeled with colloidal gold and placed on the conjugate pad. Samples containing varies amount of β-gal-192 core protein (from left to right: 0, 0.3 ng/μl, 1 ng/μl, 3 ng/μl, and 10 ng/μl) were applied on each strip. The test strips were also scanned with a LFIA reader from Qiagen.

### Example 7: Labeling of VHH Antibodies with Quantum Dots

A VHH antibody generated in project QL01 was used to test the labeling conditions with quantum dots. Labeling was done using EDC as a cross linker using QD with carboxyl functional groups. The labeled antibody was tested in lateral flow assays (FIG. 14). QL01 is a small molecule antibody and a competition immunoassay was conducted: the testing line was printed with QL01 antigen, and the control line was printed with goat anti-rabbit antibody. The conjugate pad was loaded with QD labeled anti-QL01 VHH-rFc fusion antibody.

With different concentrations of the QL01 in the sample, it competed with the QL01 on the testing line. As a result, when there was no QL01 in the sample, the labeled antibody was captured by the QL01 at the testing line to show a bright QD signal. When there was extra amount of QL01 in the sample, it would pre-occupy the QD-anti-QL01, which would not be able to bind on the test line, therefore the signal was reduced or disappeared.

### Example 8: Fusion VHH Antibodies with Llama Fc or Human Fc Domains

In this example, expression vectors with llama Fc or human Fc domains are constructed. The advantage of each fusion protein is listed in Table 4.

TABLE 4
Comparison of VHH and Fc fusion proteins from different species.

| Protein | |
|---|---|
| Configuration | Advantage |
| VHH alone | Minimal structure. Can be modified to multimeric |
| | form to improve affinity (via avidity effect). |
| Llama Fc | Natural to VHH antibody, might be more stable |
| fusion | |
| Human Fc | Same as those in human patient blood, will be less likely |
| fusion | to cause interference by host antibody (such as problems |
| | caused by human anti- mouse antibodies in |
| | many immunoassays) |
| Rabbit Fc | Wide selection of secondary antibodies (such as goat |
| fusion | anti-rabbit) and derivatives available. More convenient |
| | and versatile to use in research applications. |

### Example 9: Determination of the Sensitivity, Specificity and Reproducibility of the ELISA with Seroconversion Panels

In this example, based on the optimized results, about 50 to 100 ELISA kits are produced. Purified HCV core protein is spiked in negative human serum in various concentrations. This material also serve as a standard in ELISA kits. The detection limit of the ELISA is determined with known concentrations of HCV core protein.

Seroconversion panels are tested with the ELISA kits. Seracare currently carries 14 different panels covering genotype 1a, 2a, 2b, and 3a, with days spanning from 9 to 152. Zeptometrix offers over 80 different seroconversion panels and HCV standard samples. The NATrol Cat # NATHCV-0001, 0002, 0003, 0004, 0005 have target RNA concentrations ranging from 200 to 100,000 IU/ml, but the status of core protein is unknown. ELISA results can be compared with RNA PCR results to determine any correlation of core protein with RNA copy number.

Specificity to different genotype is preliminarily determined using the genotype panel “HCV Worldwide AccuSet Performance Panel” (Seracare #0810-0173), which is a 20-member panel of undiluted, naturally occurring plasma samples. According to the manufacturer, the Panel members represent bleeds from multiple individuals positive for HCV from varying countries of origin. Each sample represents a single collection event. This panel of human plasma samples demonstrates a diverse collection of HCV genotypes 1, 2, 3, 4, 5, and 6 with varying subtypes. Test results from commercially-available HCV genotype, RNA, and antibody assays are included for characterization of the panel members.

Reproducibility is determined with three groups of positive samples with the low, middle and high HCV protein level. The intra-assay and inter-assay coefficients of variation (CV) for the standards are determined.

### Example 10: Lateral Flow Immunoassays

In this example, colloidal gold is used as a label to develop the assays because it is more convenient and more economical. With the antibodies selected from sandwich ELISAs, both capture and detection antibodies are tested at solid phase (printed on the test line) or mobile phase (labeled with colloidal gold). Properties of antibodies are affected upon binding on the nitrocellulose or conjugation to the nanoparticle gold.

Several other factors are tested as well to optimize the lateral flow assays. Some common ones include: the ratio of gold to antibody, amount of conjugates to spray on the conjugate pad and the concentration of antibody to be printed on the test line.

In another experiment, gold labels are used for qualitative and semi-quantitative rapid tests. In another experiment, fluorescent labeled antibodies are used to improve the utility of LFIA as a diagnostic platform. Qiagen has developed a portable ESEQuant lateral flow reader which performs both colorimetric and fluorescence detection of up to 2 different wavelengths. The companion Lateral Flow Studio Software allows user to configure up to 60 different parameters. In addition, up to 15 different measurement or control parameters can be defined per test strip, and each test parameter can be freely defined. The portable reader uses a rechargeable lithium battery which can take up to 600 readings without the need of external power supply. Other convenient features include: touch screen data entry, USB ports, internal 2D barcode reader, external mobile printer, etc. The system can be used for both qualitative and quantitative assays. This system has been successfully used in LFIA development. The test result in FIG. 13 was obtained with the ESEQuant reader.

1. A polypeptide comprising:
a) an isolated hepatitis C virus (HCV) core antigen polypeptide comprising the amino acid sequence set forth in SEQ ID NO: 1; or
b) an isolated HCV core antigen fragment comprising any one of the amino acid sequences set forth in SEQ ID NOs: 2-11, or any combination thereof,
wherein said polypeptide does not comprise a full length natural HCV core antigen.

2. The polypeptide of claim 1, which is a part of a fusion polypeptide.

3. The polypeptide of claim 2, which further comprises a tag sequence.

4. The polypeptide of any of claims 1-3, which comprises or is conjugated to a detectable label.

5. The polypeptide of claim 4, wherein the detectable label is a colorimetric, a radioactive, an enzymatic, a luminescent or a fluorescent label.

6. The polypeptide of claim 4 or 5, wherein the detectable label is a soluble label or a particle (such as a nanoparticle or a microparticle) or particulate label.

7. The polypeptide of any of claims 1-6, which is attached to a solid surface, such as a blot, a membrane, a sheet, a paper, a bead, a particle (such as a nanoparticle or a microparticle), an assay plate, an array, a glass slide, a microtiter, or an ELISA plate.

8. A polynucleotide which encodes the polypeptide of any of claims 1-7, or a complimentary strand thereof.

9. The polynucleotide of claim 8, which is codon-optimized for expression in a non-human organism or a cell.

10. The polynucleotide of claim 9, wherein the organism or cell is a virus, a bacterium, a yeast cell, a plant cell, an insect cell, or a mammalian cell.

11. The polynucleotide of any of claims 7-10, wherein the polynucleotide is DNA or RNA.

12. The polynucleotide of claim 8, wherein the polynucleotide comprises the nucleotide sequence of SEQ ID NO: 12.

13. A vector comprising the polynucleotide of any of claims 8-12.

14. The vector of claim 13, wherein the polynucleotide further comprises a promoter sequence.

15. The vector of claim 13 or 14, wherein the polynucleotide further encodes a tag sequence.

16. The vector of any of claims 13-15, wherein the polynucleotide comprises a poly-A sequence.

17. The vector of any of claims 13-16, wherein the polynucleotide comprises a translation termination sequence.

18. A non-human organism or a cell transformed with the vector of any of claims 13-17.

19. The non-human organism or cell of claim 18, which is a virus, a bacterium, a yeast cell, an insect cell, a plant cell, or a mammalian cell.

20. A method of recombinantly making a polypeptide, which method comprises culturing the organism or cell of claim 19, and recovering said polypeptide from said organism or cell.

21. The method of claim 20, further comprising isolating the polypeptide, optionally by chromatography.

22. A polypeptide produced by the method of claim 20 or 21.

23. The polypeptide of claim 22, wherein the polypeptide comprises a native glycosylation pattern.

24. The polypeptide of claim 22 or 23, wherein the polypeptide comprises a native phosphorylation pattern.

25. A kit for detecting an antibody that specifically binds to an HCV core antigen polypeptide, which kit comprises, in a container, the polypeptide of any of claims 1-7 and 22-24.

26. A method for detecting an antibody that specifically binds to an HCV core antigen polypeptide in a sample, which method comprises contacting the polypeptide of claims 1-7 and 22-24 with said sample and detecting a polypeptide-antibody complex formed between the polypeptide and the HCV core antigen polypeptide in the sample to assess the presence, absence and/or amount of the antibody that specifically binds to an HCV core antigen polypeptide in the sample.

27. The method of claim 26, wherein the sample is from a mammal.

28. The method of claim 27, wherein the mammal is a human.

29. The method of claim 27 or 28, wherein the method is used for diagnosis, prognosis, stratification, risk assessment, or treatment monitoring of an HCV infection.

30. The method of any of claims 27-29, wherein the sample is selected from the group consisting of a whole blood sample, a serum, a plasma, a urine and a saliva sample.

31. The method of any of claims 27-30, wherein the sample is a clinical sample.

32. The method of any of claims 27-31, wherein the polypeptide-antibody complex is assessed by a sandwich or competitive assay format, optionally with a binder or antibody.

33. The method of claim 32, wherein the binder or antibody is attached to a surface and functions as a capture binder or antibody.

34. The method of claim 32 or 33, wherein at least one of the binders or antibodies is labeled.

35. The method of any of claims 27-34, wherein the polypeptide-antibody complex is assessed by a format selected from the group consisting of an enzyme-linked immunosorbent assay (ELISA), immunoblotting, immunoprecipitation, radioimmunoassay (RIA), immunostaining, latex agglutination, indirect hemagglutination assay (IHA), complement fixation, indirect immunofluorescent assay (IFA), nephelometry, flow cytometry assay, plasmon resonance assay, chemiluminescence assay, lateral flow immunoassay, μ-capture assay, inhibition assay and avidity assay.

36. The method of any of claims 27-34, wherein the polypeptide-antibody complex is assessed in a homogeneous or a heterogeneous assay format.

37. An isolated camelid antibody that specifically binds to an epitope within an HCV core antigen polypeptide.

38. The isolated camelid antibody of claim 37, which is derived from a camel, a llama, an alpaca (Vicugna pacos), a vicuña (Vicugna vicugna), or a guanaco (Lama guanicoe).

39. The isolated camelid antibody of claim 38, wherein the camel is a dromedary camel (Camelus dromedarius), a Bactrian camel (Camelus bactrianus), or a wild Bactrian camel (Camelus ferus).

40. The isolated camelid antibody of any of claims 37-39, wherein the antibody is a polyclonal antibody, a monoclonal antibody, an antibody fragment or a single-domain heavy-chain (VHH) antibody.

41. The isolated camelid antibody of claim 40, wherein the VHH antibody is a llama VHH antibody.

42. The isolated camelid antibody of any of claims 37-41, wherein the antibody specifically binds to an epitope within an HCV core antigen polypeptide from a genotype selected from the group consisting of 1, 1a, 1a/1b, 1b, 2, 2a, 2a/2c, 2b, 3a, 3k, 4, 4a, 4a/4c, 4c/4d, 4c/4d/4e, 5/5a, 6a, and 6i.

43. The isolated camelid antibody of any of claims 37-41, wherein the antibody specifically binds to an epitope within the polypeptide comprising the amino acid sequence set forth in SEQ ID NO: 1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, or SEQ ID NO:11, or any combination thereof.

44. The isolated camelid antibody of claim 43, which is produced by a process that comprises the steps of:
a) immunizing a camelid with a polypeptide comprising the amino acid sequence set forth in SEQ ID NO: 1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, or SEQ ID NO:11, or any combination thereof; and
b) recovering the antibody from the camelid.

45. The isolated camelid antibody of claim 44, wherein the camelid is a llama.

46. The isolated camelid antibody of any of claims 37-45, wherein the antibody specifically binds to the HCV core antigen polypeptide.

47. The isolated camelid antibody of any of claims 37-46, which is a part of a fusion polypeptide.

48. The isolated camelid antibody of claim 47, wherein the fusion polypeptide comprises a variable region of a camelid antibody and a constant region of a non-camelid antibody.

49. The isolated camelid antibody of claim 48, wherein the fusion polypeptide comprises a variable region of a llama antibody and a constant region of a non-camelid antibody.

50. The isolated camelid antibody of claim 49, wherein the fusion polypeptide comprises a variable region of a llama antibody and a constant region of a rabbit antibody.

51. The isolated camelid antibody of claim 50, wherein the fusion polypeptide is a fusion llama VHH antibody that comprises a variable region of the llama VHH antibody and a Fc region of a rabbit antibody.

52. The isolated camelid antibody of any of claims 37-51, which is a humanized antibody.

53. The isolated camelid antibody of any of claims 37-52, which is conjugated to a detectable label.

54. The isolated camelid antibody of claim 53, wherein the detectable label is a colorimetric, a radioactive, an enzymatic, a luminescent or a fluorescent label.

55. The isolated camelid antibody of claim 53 or 54, wherein the detectable label is a soluble label or a particle (such as a nanoparticle or a microparticle) or particulate label.

56. The isolated camelid antibody of any of claims 37-55, which is attached to a solid surface, such as a blot, a membrane, a sheet, a paper, a bead, a particle (such as a nanoparticle or a microparticle), an assay plate, an array, a glass slide, a microtiter, or an ELISA plate.

57. A method for detecting an HCV core antigen polypeptide in a sample, which method comprises contacting the HCV core antigen polypeptide in the sample with an isolated camelid antibody of any of claims 37-56, and detecting a polypeptide-antibody complex formed between the HCV core antigen polypeptide in the sample and the isolated camelid antibody to assess the presence, absence and/or amount of the HCV core antigen polypeptide in the sample.

58. The method of claim 57, wherein the sample is from a subject, e.g., a mammal.

59. The method of claim 58, wherein the mammal is a human.

60. The method of any of claims 57-59, wherein the method is used for diagnosis, prognosis, stratification, risk assessment, or treatment monitoring of an HCV infection.

61. The method of any of claims 57-59, wherein the method is used for identifying HCV infection in a seronegative mammal, identifying a seropositive mammal that is actively infected with HCV, or for monitoring an anti-HCV therapy.

62. The method of any of claims 57-61, wherein the sample is selected from the group consisting of a whole blood sample, a serum, a plasma, a urine and a saliva sample.

63. The method of any of claims 57-62, wherein the sample is a clinical sample.

64. The method of any of claims 57-63, wherein the polypeptide-antibody complex is assessed by a sandwich or competitive assay format.

65. The method of claim 64, wherein the camelid antibody is attached to a surface and functions as a capture antibody.

66. The method of claim 64, wherein the camelid antibody is labeled.

67. The method of claim 64, wherein the polypeptide-antibody complex is assessed by a sandwich assay format that uses two camelid antibodies, one being a capture antibody and the other being a labeled antibody.

68. The method of any of claims 57-67, wherein the polypeptide-antibody complex is assessed by a format selected from the group consisting of an enzyme-linked immunosorbent assay (ELISA), immunoblotting, immunoprecipitation, radioimmunoassay (RIA), immunostaining, latex agglutination, indirect hemagglutination assay (IHA), complement fixation, indirect immunofluorescent assay (IFA), nephelometry, flow cytometry assay, plasmon resonance assay, chemiluminescence assay, lateral flow immunoassay, μ-capture assay, inhibition assay and avidity assay.

69. The method of any of claims 57-67, wherein the polypeptide-antibody complex is assessed in a homogeneous or a heterogeneous assay format.

70. The method of any of claims 57-69, which further comprises disassociating the HCV core antigen polypeptide in the sample from an antibody of the subject to be tested.

71. The method of claim 70, wherein the HCV core antigen polypeptide in the sample is disassociated from the antibody of the subject to be tested by changing the pH of the sample to be 4 or lower, or to be 9 or higher, by treating the sample with a protein denaturing agent, and/or by heating the sample to between about 35° C. and about 95° C., preferably to between about 45° C. and about 70° C., concurrently with or before contacting the sample with the camelid antibody.

72. The method of claim 71, wherein the protein denaturing agent is guanidine hydrochloride (e.g., about 1 M to about 6 M), guanidinium thiocyanate (e.g., about 1 M to about 6 M), SDS (e.g., about 0.1% to about 2%), β-mercaptoethanol, DTT or other reducing agent for disulfide bond disruption at various concentrations, or urea (e.g., about 2 M to about 8 M), or any combination thereof.

73. The method of claim 71 or 72, which further comprises adjusting the pH of the sample to between about 6 and about 8, and/or removing the protein denaturing agent concurrently with or before contacting the sample with the camelid antibody.

74. The method of claim 71 or 72, wherein the camelid antibody is a camelid VHH antibody, and the sample is contacted with the camelid VHH antibody at a pH that is at 4 or lower, or at 9 or higher, and/or in the presence of the protein denaturing agent.

75. The method of claim 74, wherein the camelid VHH antibody is a llama VHH antibody.

76. A kit for detecting an HCV core antigen polypeptide, which kit comprises, in a container, an isolated camelid antibody of any of claims 37-56.

77. A lateral flow device comprising a matrix that comprises an isolated camelid antibody of any of claims 37-56 immobilized on a test site on the matrix downstream from a sample application site on the matrix.

78. The lateral flow device of claim 77, which further comprises a labeled camelid antibody of any of claims 37-56 on the matrix upstream from the test site, said labeled camelid antibody being capable of moved by a liquid sample and/or a further liquid to the test site and/or a control site to generate a detectable signal.

79. A method for detecting an HCV core antigen polypeptide in a liquid sample, which method comprises:
a) contacting a liquid sample with the lateral flow device of claim 77 or 78, wherein the liquid sample is applied to a site of the lateral flow device upstream of the test site;
b) transporting an HCV core antigen polypeptide, if present in the liquid sample, and a labeled camelid antibody of any of claims 37-56 to the test site; and
c) assessing the presence, absence, and/or amount of a signal generated by the labeled camelid antibody at the test site to determining the presence, absence and/or amount of the HCV core antigen polypeptide in the liquid sample.

80. A method for detecting an analyte in a sample from a subject, which method comprises:
a) disassociating an analyte in a sample from a subject that is bound to an antibody of the subject from the antibody of the subject by changing the pH of the sample to be 4 or lower, or to be 9 or higher, by treating the sample with a protein denaturing agent, and/or by heating the sample to between about 35° C. and about 95° C., preferably to between about 45° C. and about 70° C.;
b) contacting the analyte disassociated from the antibody of the subject with a camelid VHH antibody at a pH that is at 4 or lower, or at 9 or higher, and/or in the presence of the protein denaturing agent, and/or at a temperature of about 35° C. and about 95° C., preferably about 45° C. and about 70° C., and detecting an analyte-antibody complex formed between the disassociated analyte and the camelid antibody to assess the presence, absence and/or amount of the analyte in the sample.

81. The method of claim 80, wherein the analyte is selected from the group consisting of a cell, a cellular organelle, a virus, a molecule and an aggregate or complex thereof.

82. The method of claim 81, wherein the cell is selected from the group consisting of an animal cell, a plant cell, a fungus cell, a bacterium cell, a recombinant cell and a cultured cell.

83. The method of claim 81, wherein the cellular organelle is selected from the group consisting of a nucleus, a mitochondrion, a chloroplast, a ribosome, an ER, a Golgi apparatus, a lysosome, a proteasome, a secretory vesicle, a vacuole and a microsome.

84. The method of claim 81, wherein the molecule is selected from the group consisting of an inorganic molecule, an organic molecule and a complex thereof.

85. The method of claim 84, wherein the inorganic molecule is an ion selected from the group consisting of a sodium, a potassium, a magnesium, a calcium, a chlorine, an iron, a copper, a zinc, a manganese, a cobalt, an iodine, a molybdenum, a vanadium, a nickel, a chromium, a fluorine, a silicon, a tin, a boron and an arsenic ion.

86. The method of claim 84, wherein the organic molecule is selected from the group consisting of an amino acid, a peptide, a protein, a polypeptide, a nucleoside, a nucleotide, an oligonucleotide, a nucleic acid, a polynucleotide, a vitamin, a monosaccharide, an oligosaccharide, a polysaccharide a carbohydrate, a lipid and a complex thereof.

87. The method of any of claims 80-86, wherein the analyte is a marker for a disease, disorder or infection.

88. The method of claim 87, wherein the method is used for diagnosis, prognosis, stratification, risk assessment, or treatment monitoring of the disease, disorder or infection.

89. The method of claim 87, wherein the analyte is a marker for is bacterial or viral infection.

90. The method of claim 89, wherein the analyte is a marker for HCV infection.

91. The method of claim 90, wherein the analyte is an HCV polypeptide.

92. The method of claim 91, wherein the HCV polypeptide is an HCV core antigen polypeptide.

93. The method of any of claims 90-92, wherein the method is used for diagnosis, prognosis, stratification, risk assessment, or treatment monitoring of an HCV infection.

94. The method of any of claims 90-92, wherein the method is used for identifying HCV infection in a seronegative mammal, identifying a seropositive mammal that is actively infected with HCV, or for monitoring an anti-HCV therapy.

95. The method of any of claims 80-94, wherein the subject is a mammal.

96. The method of claim 95, wherein the mammal is a human.

97. The method of any of claims 80-96, wherein the sample is selected from the group consisting of a whole blood sample, a serum, a plasma, a urine and a saliva sample.

98. The method of any of claims 80-97, wherein the sample is a clinical sample.

99. The method of any of claims 80-98, wherein the analyte is disassociated from the antibody of the subject by changing the pH of the sample to be 4 or lower, or wherein the analyte is disassociated from the antibody of the subject by changing the pH of the sample to be 9 or higher.

100. The method of any of claims 80-98, wherein the analyte is disassociated from the antibody of the subject by heating the sample to between about 35° C. and about 95° C., preferably to between about 45° C. and about 70° C.

101. The method of any of claims 80-98, wherein the analyte is disassociated from the antibody of the subject by treating the sample with a protein denaturing agent.

102. The method of claim 95, wherein the protein denaturing agent is guanidine hydrochloride (e.g., about 1 M to about 6 M), guanidinium thiocyanate (e.g., about 1 M to about 6 M), SDS (e.g., about 0.1% to about 2%), β-mercaptoethanol, DTT or other reducing agent for disulfide bond disruption at various concentrations, or urea (e.g., about 2 M to about 8 M), or any combination thereof.

103. The method of any of claims 80-98, wherein the analyte is disassociated from the antibody of the subject by changing the pH of the sample to be 4 or lower, by treating the sample with a protein denaturing agent, and/or by heating the sample to between about 35° C. and about 95° C., preferably to between about 45° C. and about 70° C.

104. The method of any of claims 80-98, wherein the analyte is disassociated from the antibody of the subject by changing the pH of the sample to be 9 or higher, by treating the sample with a protein denaturing agent, and/or by heating the sample to between about 35° C. and about 95° C., preferably to between about 45° C. and about 70° C.

105. The method of any of claims 80-104, wherein the analyte disassociated from the antibody of the subject is contacted with the camelid VHH antibody at a pH that is at 4 or lower, or wherein the analyte disassociated from the antibody of the subject is contacted with the camelid VHH antibody at pH that is at 9 or higher.

106. The method of any of claims 80-104, wherein the analyte disassociated from the antibody of the subject is contacted with the camelid VHH antibody at a temperature of about 35° C. and about 95° C., preferably about 45° C. and about 70° C.

107. The method of any of claims 80-104, wherein the analyte disassociated from the antibody of the subject is contacted with the camelid VHH antibody in the presence of the protein denaturing agent.

108. The method of claim 107, wherein the protein denaturing agent is guanidine hydrochloride (e.g., about 1 M to about 6 M), guanidinium thiocyanate (e.g., about 1 M to about 6 M), SDS (e.g., about 0.1% to about 2%), β-mercaptoethanol, DTT or other reducing agent for disulfide bond disruption at various concentrations, or urea (e.g., about 2 M to about 8 M), or any combination thereof.

109. The method of any of claims 80-104, wherein the analyte disassociated from the antibody of the subject is contacted with the camelid VHH antibody at a pH that is at 4 or lower and in the presence of the protein denaturing agent.

110. The method of any of claims 80-104, wherein the analyte disassociated from the antibody of the subject is contacted with the camelid VHH antibody at pH that is at 9 or higher and in the presence of the protein denaturing agent.

111. The method of any of claims 80-110, wherein the camelid VHH antibody is a llama VHH antibody.

112. The camelid antibody of claim 111, wherein the llama VHH antibody is a fusion llama VHH antibody that comprises a variable region of the llama VHH antibody and a constant region of a rabbit antibody.

113. The method of any of claims 80-112, wherein the analyte-antibody complex is assessed by a sandwich or competitive assay format.

114. The method of claim 113, wherein the camelid antibody is attached to a surface and functions as a capture antibody.

115. The method of claim 113, wherein the camelid antibody is labeled.

116. The method of claim 113, wherein the analyte-antibody complex is assessed by a sandwich assay format that uses two camelid antibodies, one being a capture antibody and the other being a labeled antibody.

117. The method of any of claims 80-116, wherein the analyte-antibody complex is assessed by a format selected from the group consisting of an enzyme-linked immunosorbent assay (ELISA), immunoblotting, immunoprecipitation, radioimmunoassay (RIA), immunostaining, latex agglutination, indirect hemagglutination assay (IHA), complement fixation, indirect immunofluorescent assay (IFA), nephelometry, flow cytometry assay, plasmon resonance assay, chemiluminescence assay, lateral flow immunoassay, μ-capture assay, inhibition assay and avidity assay.

118. The method of any of claims 80-116, wherein the analyte-antibody complex is assessed in a homogeneous or a heterogeneous assay format.

119. The method of claim 116, wherein the analyte-antibody complex is assessed by a lateral flow sandwich assay format that uses two camelid antibodies, one being a capture antibody and the other being a labeled antibody.

120. The method of claim 119, wherein the labeled antibody is labeled with a particle (such as a nanoparticle or a microparticle) or particulate label.

121. The method of any of claims 80-120, wherein the steps a) and b) are conducted concurrently.

122. The method of any of claims 80-120, wherein the step a) is conducted before the step b).

123. The method of any of claims 80-122, which is conducted to assess the presence or absence of the analyte in the sample.

124. The method of any of claims 80-122, which is conducted to assess the amount of the analyte in the sample.


SEQUENCE LISTING (16 sequences)

SEQ ID NO: 1 - protein, 191 aa, Hepatitis C virus
MSTNPKPQRKTKRNTNRRPQDVKFPGGGQIVGGVYLLPRRGPRLGVRATRKTSERSQPRGRRQPIPKARQPEGRAWAQPGYPWPLYGNEGMGWAGWLLSPRGSRPSWGPTDPRRRSRNLGKVIDTLTCGFADLMGYIPLVGAPLGGAARALAHGVRVLEDGVNYATGNLPGCSFSIFLLALLSCLTIPASA

SEQ ID NO: 2 - protein, 10 aa, Hepatitis C virus
MSTNPKPQRC

SEQ ID NO: 3 - protein, 10 aa, Hepatitis C virus
TKRNTNRRPC

SEQ ID NO: 4 - protein, 18 aa, Hepatitis C virus
DVKFPGGGQIVGGVYCRR

SEQ ID NO: 5 - protein, 14 aa, Hepatitis C virus
CLLPRRGPRLGVRA

SEQ ID NO: 6 - protein, 19 aa, Hepatitis C virus
CRKTSERSQPRGRRQPIPK

SEQ ID NO: 7 - protein, 15 aa, Hepatitis C virus
RKRCWAQPGYPWPLY

SEQ ID NO: 8 - protein, 13 aa, Hepatitis C virus
RKRCGWAGWLLSP

SEQ ID NO: 9 - protein, 18 aa, Hepatitis C virus
DPRRRSRNLGKVIDTLTC

SEQ ID NO: 10 - protein, 19 aa, Hepatitis C virus
RKRCGFADLMGYIPLVGAP

SEQ ID NO: 11 - protein, 15 aa, Hepatitis C virus
EDGVNYATGNLPGCK

SEQ ID NO: 12 - DNA, 573 bp, Hepatitis C virus
atgtccacgaacccaaagccgcagcgtaaaaccaaacgtaacaccaaccgtcgcccgcaggacgtcaaattccctggtggcggtcagatcgtgggtggcgtgtacctgctgccgcgtcgtggtccgcgcctgggtgtacgtgcaacccgtaaaacctccgagcgttcccagccgcgcggtcgtcgtcagcctatccctaaagctcgtcagccggaaggtcgcgcatgggcacaaccgggttacccgtggccactgtacggtaacgaaggtatgggctgggcgggttggctgctgagcccacgtggttctcgtccgtcttggggtccgactgacccgcgtcgtcgctctcgcaatctgggtaaagttatcgacaccctgacctgcggcttcgcggatctgatgggctatatcccgctggtaggcgccccgctgggcggcgcagctcgcgctctggctcacggcgttcgcgttctggaagatggcgttaactatgcgactggcaacctgccgggctgtagcttttccattttcctgctggcgctgctgagctgcctgactattccggcgtccgcg

SEQ ID NO: 13 - protein, 180 aa, Hepatitis C virus
MSTNPKPQRKTKRNTNRRPQDVKFPGGGQIVGGVYLLPRRGPRLGVRAARKTSERSQPRGRRQPIPKDRRSTGKSWGKPGYPWPLYGNEGLGWAGWLLSPRGSRPSWGPTDPRHRSRNVGKVIDTLTCGFADLMGYIPVVGAPLGGVARALAHGVRVLEDGVNFATGNLPGCSFSIFLLA

SEQ ID NO: 14 - protein, 180 aa, Hepatitis C virus
MSTNPKPQRKTKRNTNRRPQDVKFPGGGQIVGGVYLLPRRGPRLGVRAPRKTSERSQPRGRRQPIPKDRRSTGKSWGKPGYPWPLYGNEGCGWAGWLLSPRGSRPTWGPTDPRHRSRNLGRVIDTITCGFADLMGYIPVVGAPVGGVARALAHGVRVLEDGVNYATGNLPGCSFSIFLLA

SEQ ID NO: 15 - protein, 180 aa, Hepatitis C virus
MSTNPKPQRKTKRNTNRRPQDVKFPGGGQIVGGVYLLPRRGPRLGVRATRKTSERSQPRGRRQPIPKARRPEGRAWAQPGYPWPLYGNEGLGWAGWLLSPRGSRPSWGPTDPRRRSRNLGKVIDTLTCGFADLMGYIPLVGAPLGGAARALAHGVRVLEDGVNYATGNLPGCPFSVFLLA

SEQ ID NO: 16 - protein, 180 aa, Hepatitis C virus
MSTNPKPQRKTKRNTNRRPQDVKFPGGGQIVGGVYLLPRRGPRLGVRATRKTSERSQPRGRRQPIPKARRPEGRAWAQPGYPWPLYGNEGCGWAGWLLSPRGSRPSWGPTDPRRRSRNLGKVIDTLTCGFADLMGYIPLVGAPLGGAARALAHGVRVLEDGVNYATGNLPGCSFSIFLLA